(19) United States
(12) Patent Application Publication
(10) Pub. No.: US 2020/0308149 A1
(43) Pub. Date: Oct. 1, 2020

(54) FUSED AND CROSS-LINKABLE IONIC HOLE TRANSPORT MATERIALS FOR PEROVSKITE SOLAR CELLS

(71) Applicant: Energy Everywhere, Inc., Berkeley, CA (US)

(72) Inventor: Brent POLISHAK, Oakland, CA (US)

(73) Assignee: Energy Everywhere, Inc., Berkeley (US)

(21) Appl. No.: 16/903,202

(22) Filed: Jun. 16, 2020

Related U.S. Application Data
(63) Continuation of application No. PCT/US18/66880, filed on Dec. 20, 2018.
(60) Provisional application No. 62/636,329, filed on Feb. 28, 2018.
(60) Provisional application No. 62/609,993, filed on Dec. 22, 2017.

(51) Int. Cl. C07D 403/10; H01L 51/00; C07D 405/14; C07D 403/14; C07D 233/58; H01L 33/14

(52) U.S. Cl. CPC C07D 403/10; H01L 51/0061; H01L 51/0067; H01L 51/0072; H01L 51/44; C07D 405/14; C07D 403/14; C07D 233/58; H01L 33/14; H01L 51/0073

(57) ABSTRACT

Described are compounds and mixtures that are useful as hole transport layers of photovoltaic devices, such as perovskite solar cells. The compounds and mixtures include non-lithium containing or lithium-free electrolytes, such as imidazolium-based electrolytes, and small-molecule hole transport structures, such as N,N-di-p-methoxy phenyl amine-based structures. The hole transport structures and electrolytes may be covalently bonded or may be separate molecules. The hole transport structures and electrolytes may include cross-linkable groups and may be cross-linked. Devices employing the compounds and mixtures as hole transport layers are also described, such as photovoltaic devices. Synthetic methods of making small-molecule hole transport compounds are also described.

### CROSS-REFERENCE TO RELATED APPLICATIONS

This application is a continuation of PCT Application No. PCT/US18/66880, filed Dec. 20, 2018 which claims the benefit of and priority to U.S. Provisional Application No. 62/609,993, filed on Dec. 22, 2017, and U.S. Provisional Application No. 62/636,329, filed on Feb. 28, 2018, which are hereby incorporated by reference in their entireties for all purposes.

### BACKGROUND

Small molecules have been explored as hole transport materials, including spiro-OMeTAD (2,2′,7,7′-tetrakis-(N,N′-di-p-methoxyphenlamine)-(9,9′-spirobifluorene)). For example, PCT International Application Publication No. WO 2016/139570, hereby incorporated by reference, describes some small molecule hole transport materials for optoelectronic and photoelectrochemical devices. In addition, Vivo et al. (Materials 2017, 10, 1087; doi:10.3390/ma10091087), hereby incorporated by reference, describes other hole transport materials for printable solar cells. The hole transport materials described above are not optimized and further development in this area is needed.

### SUMMARY

The present invention relates generally to compounds and mixtures useful as hole transport materials, methods of making the compounds and mixtures, methods of using the compounds and mixtures, and devices incorporating the compounds and mixtures. More particularly, disclosed embodiments provide compounds useful as hole transport materials that include a lithium-free electrolyte component covalently bonded to a hole transport structure and mixtures including cross-linkable lithium-free electrolytes components and hole transport compounds, which may also be cross-linkable. The compounds and mixtures may be cross-linked, such as by exposure to ultraviolet light, visible light, infrared light, and/or heat. Photovoltaic devices employing these compounds and mixtures, in both cross-linked and non-cross-linked forms, are also disclosed.

In an aspect, compounds are provided herein, such as compounds useful as hole transport materials. In an embodiment, a compound of this aspect has, a formula: HTS-E-R¹, where E is a lithium-free electrolyte having an anion component and a cation component, the cation component covalently bonded to HTS and R¹; HTS is a hole transport structure; R¹ is HTS; or H; or R²; or a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused; and R² is a reactive cross-linking group. In embodiments, R¹ is an organic or hetero-organic group. Optionally, R¹ is a fluorinated organic group.

In embodiments, the cation component comprises an imidazolium group. Optionally, the imidazolium group is covalently bonded to HTS and/or R¹ by one or more linking groups. Example linking groups include, but are not limited to phenyl groups, organic groups, and fluorinated organic groups. Optionally, the imidazolium group is substituted with an organic group or hetero-organic group. In embodiments, the imidazolium group is substituted with a fluorinated organic group.

In embodiments, the anion component comprises a sulfonimide or other anion group, optionally substituted with alkyl or fluoroalkyl group. For example, specific anion components include, but are not limited to

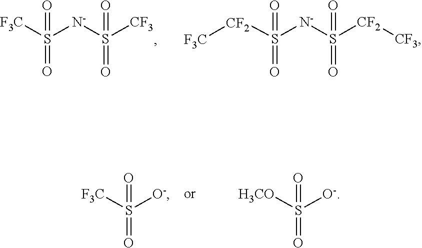

It will be appreciated that, in embodiments, the anion component is ionically bound to the cation component.

Example HTS groups may exhibit properties providing utility as a hole transport structure. For example, in embodiments, HTS is an organic or heterorganic group having a band gap of between 1.4 eV and 3.5 eV, or an ionization potential of between 4.5 eV and 5.5 eV. Optionally, HTS is a monovalent group comprising one or more homocyclic, heterocyclic, aromatic, or heteroaromatic substituents that are fused or unfused. Example heterocyclic substituents include at least one of oxygen, sulfur, selenium, tellurium, nitrogen, phosphorus, silicon, germanium, boron, aluminum, a transition metal, or a transition metal oxide. Example aromatic or heteroaromatic substituents include one or more of a phenyl, a fused phenyl, a heterocycle, or a fused heterocycle. Optionally, at least one substituent of HTS comprises triarylamine, carbazole, furan, thiophene, pyridine, or combinations thereof.

In some embodiments, HTS includes one or more phenyl or substituted phenyl groups, such as including one or more organic substituents or reactive cross-linking substituents. In a specific embodiment, HTS includes a substituted diphenyl amino group. In a specific embodiment, HTS includes a substituted carbazole. These embodiments may be combined, such as where HTS comprises a diphenylamino substituted carbazole.

In specific embodiments, HTS-E-R¹ comprises

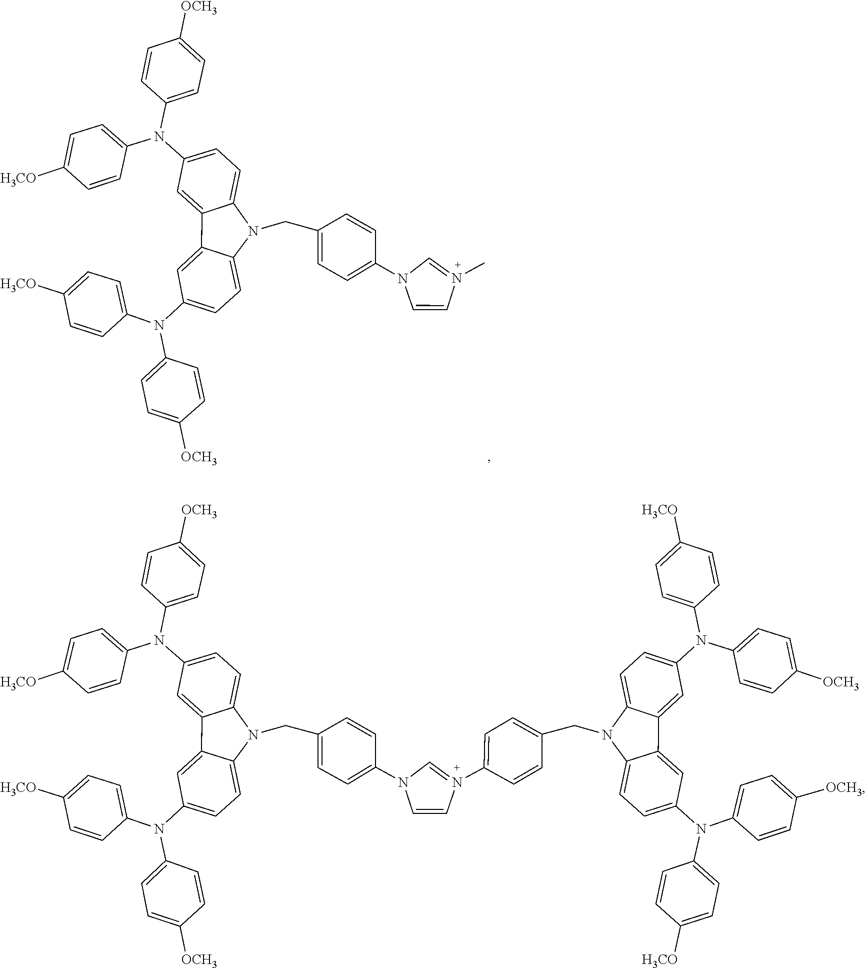

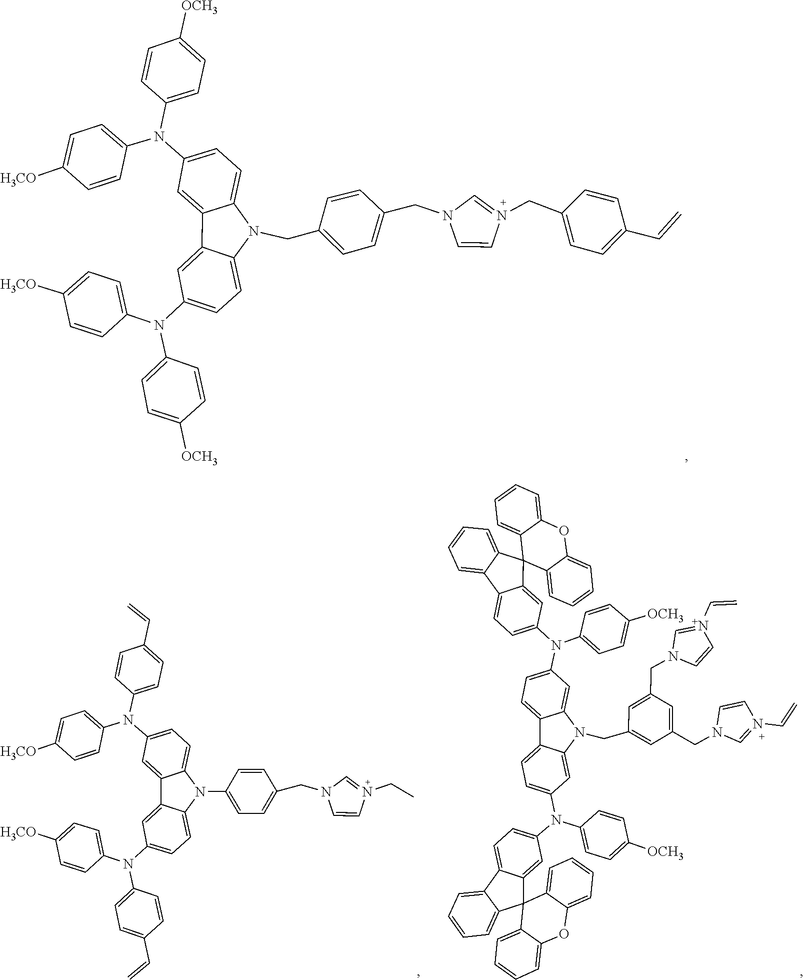

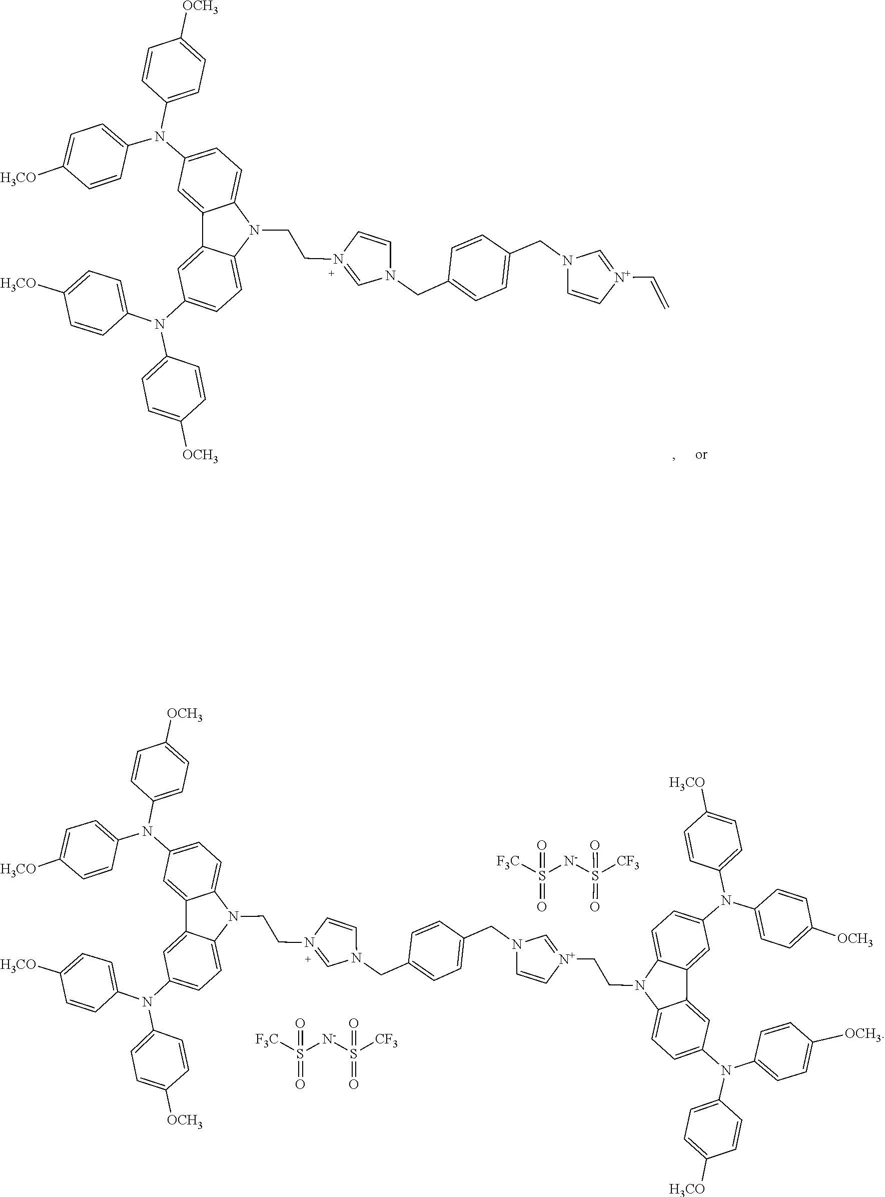

In specific embodiments, compounds of this aspect have the formula

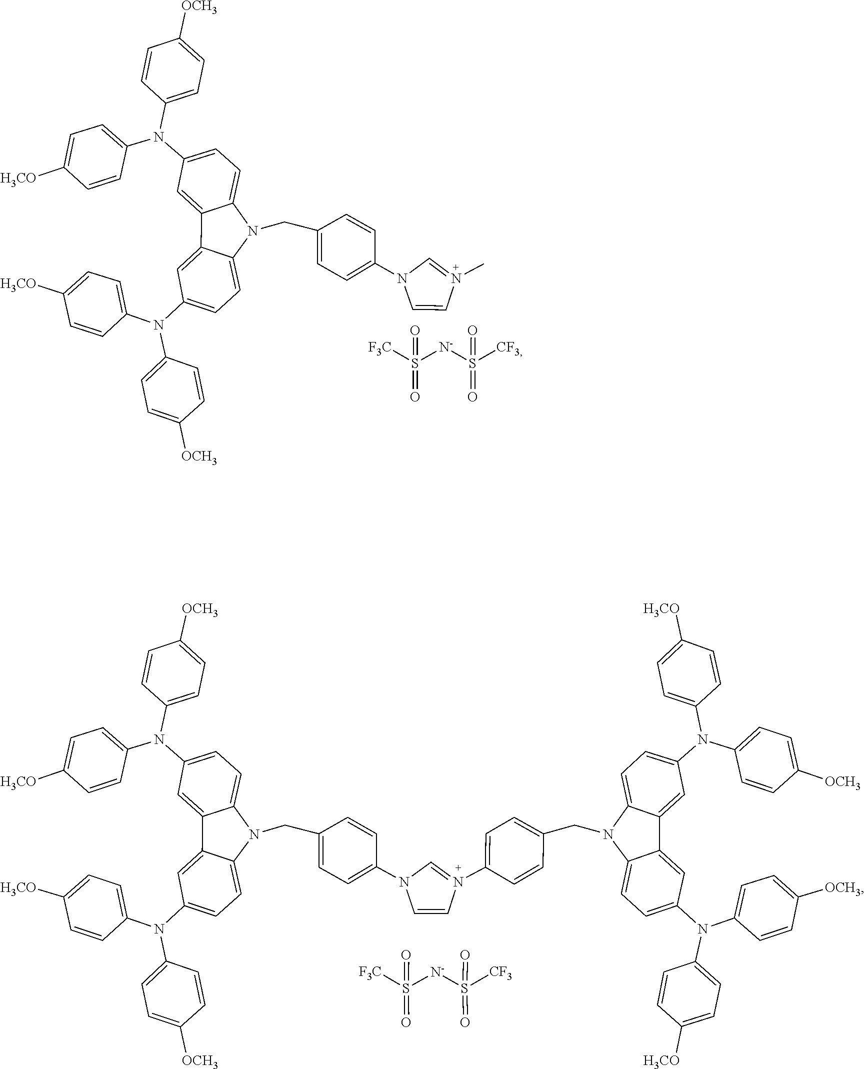 [SMILES: COc1ccc(N(c2ccc(OC)cc2)c2ccc3c(c2)c2cc(N(c4ccc(OC)cc4)c4ccc(OC)cc4)ccc2n3Cc2ccc(-n3cc[n+](C)c3)cc2)cc1 ; O=S(=O)([N-]S(=O)(=O)C(F)(F)F)C(F)(F)F ; COc1ccc(N(c2ccc(OC)cc2)c2ccc3c(c2)c2cc(N(c4ccc(OC)cc4)c4ccc(OC)cc4)ccc2n3Cc2ccc(-n3cc[n+](-c4ccc(Cn5c6ccc(N(c7ccc(OC)cc7)c7ccc(OC)cc7)cc6c6cc(N(c7ccc(OC)cc7)c7ccc(OC)cc7)ccc65)cc4)c3)cc2)cc1 ; O=S(=O)([N-]S(=O)(=O)C(F)(F)F)C(F)(F)F]

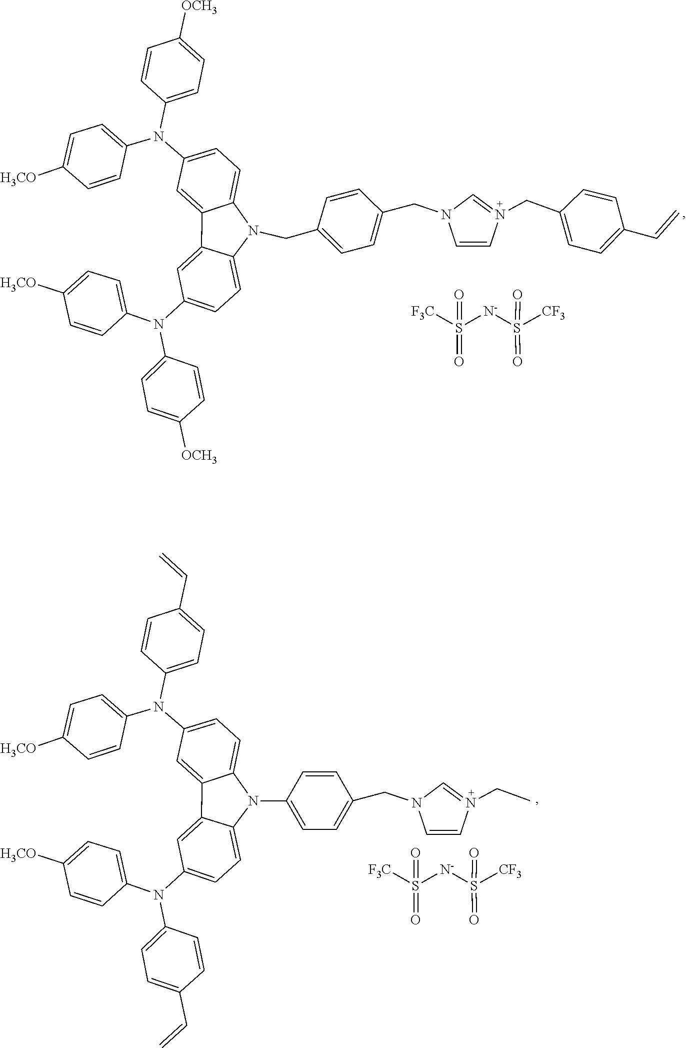 [SMILES: C=Cc1ccc(C[n+]2ccn(Cc3ccc(Cn4c5ccc(N(c6ccc(OC)cc6)c6ccc(OC)cc6)cc5c5cc(N(c6ccc(OC)cc6)c6ccc(OC)cc6)ccc54)cc3)c2)cc1 ; O=S(=O)([N-]S(=O)(=O)C(F)(F)F)C(F)(F)F ; C=Cc1ccc(N(c2ccc(OC)cc2)c2ccc3c(c2)c2cc(N(c4ccc(C=C)cc4)c4ccc(OC)cc4)ccc2n3-c2ccc(Cn3cc[n+](CC)c3)cc2)cc1 ; O=S(=O)([N-]S(=O)(=O)C(F)(F)F)C(F)(F)F]

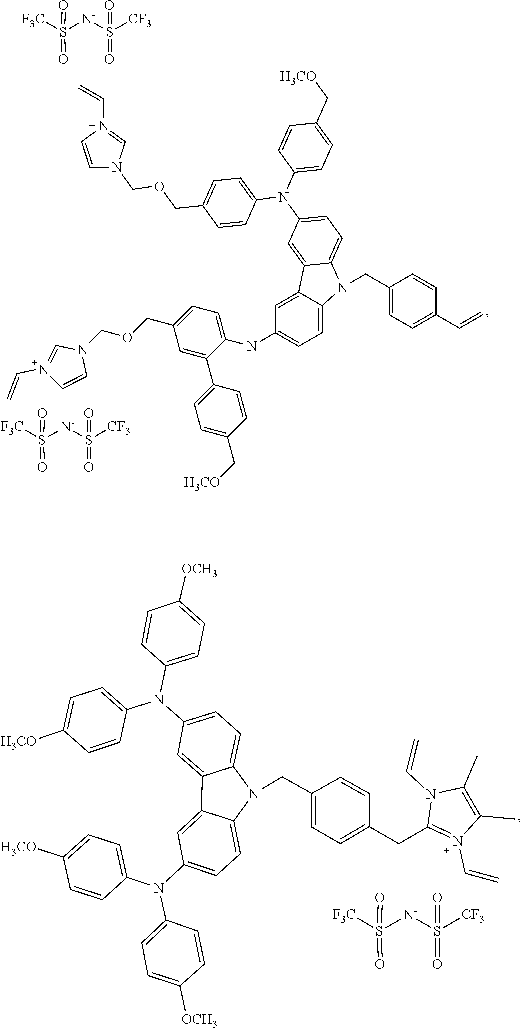

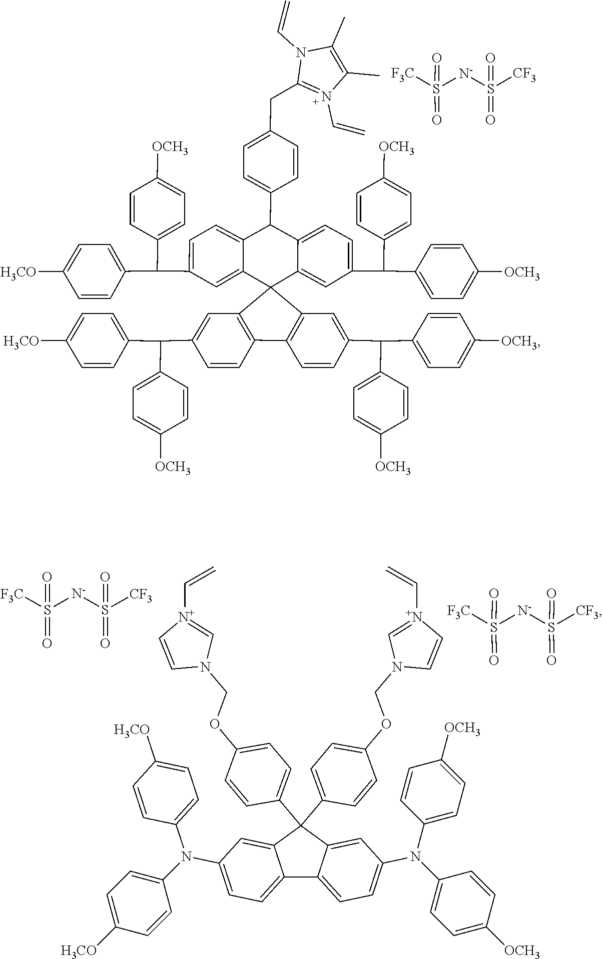

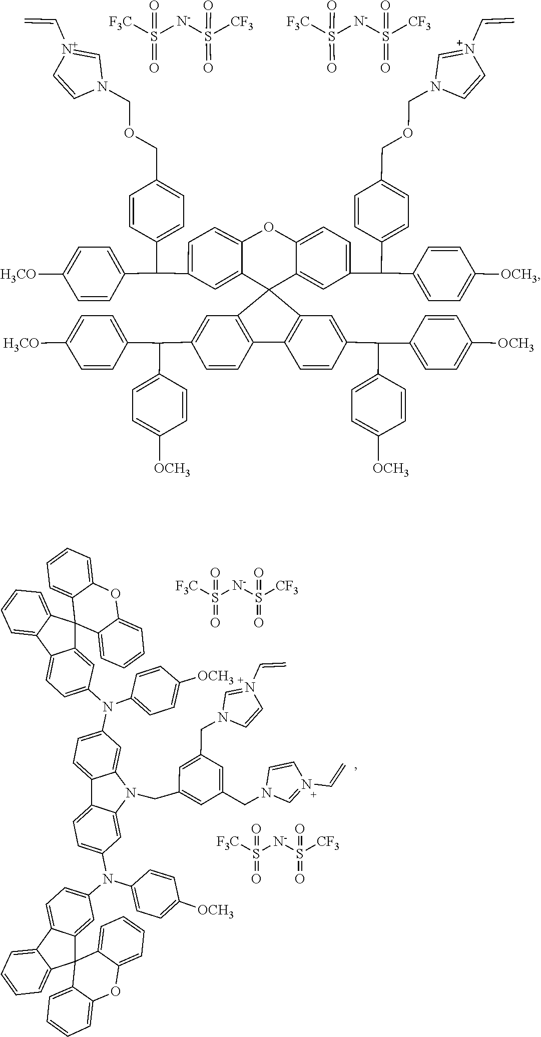

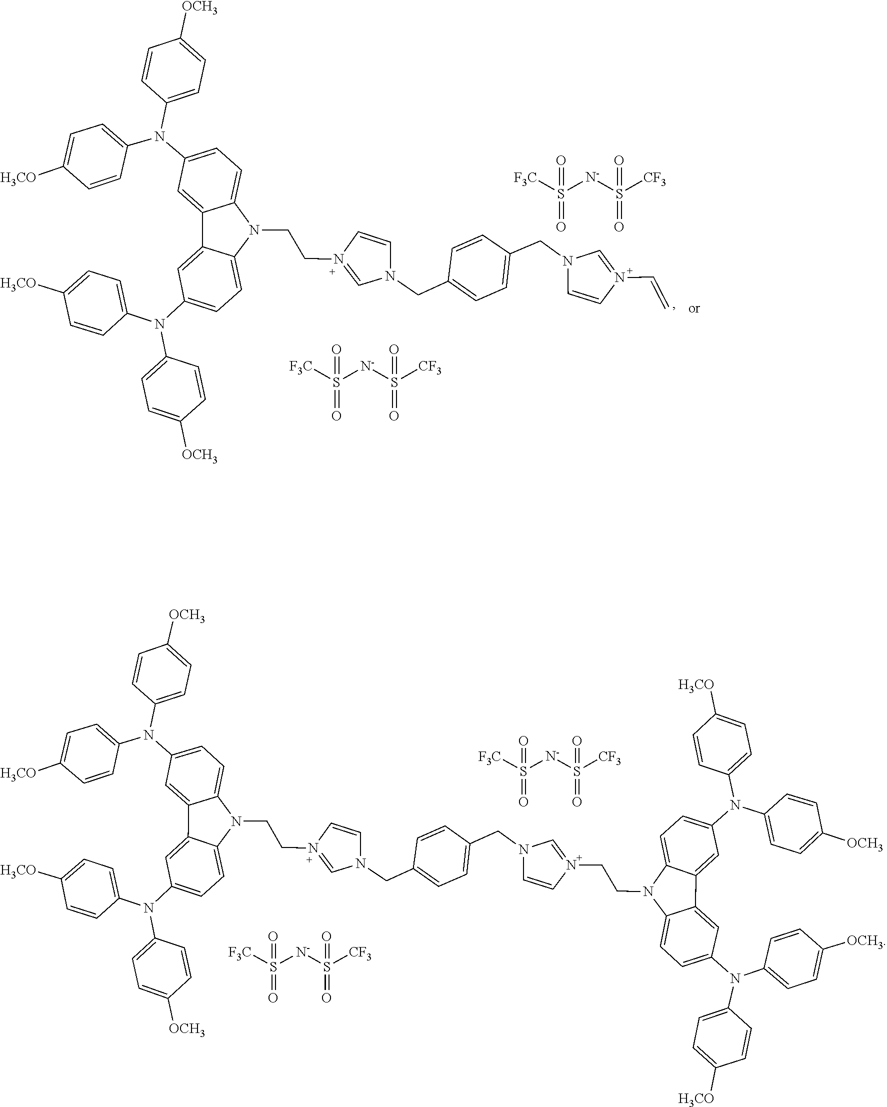

As described herein, compounds of this aspect are useful as materials of a hole transport layer, such as a hole transport layer comprising the compound dissolved in a solvent. Example solvents include polar solvents. Optionally, molecules of the compound are distributed throughout the solvent. Optionally, molecules of the compound do not phase separate from the solvent. Optionally, molecules of the compound form a packed or stacked morphology with one another.

In a related aspect, mixtures are provided, such as mixtures including hole transport compounds and a lithium free electrolyte. Hole transport compounds may optionally include those described above. In a specific embodiment, a mixture of this aspect comprises a lithium-free electrolyte; and a cross-linkable hole transport compound. Optionally, the cross-linkable hole transport compound has a formula: HTS-L³-R³, where L³ is a spacer substituent selected from the group including a C₁-C₂₀ branched, unbranched, cyclic, or polycyclic bivalent aliphatic group that is saturated or unsaturated and substituted or unsubstituted; and a bivalent substituted or unsubstituted aromatic group that is fused or unfused; and a second lithium-free electrolyte having a group covalently bonded to HTS and R³; HTS is a cross-linkable hole transport structure; and R³ is HTS, H; a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused. Optionally, the mixture further comprises a second cross-linkable hole transport compound independently having a formula HTS-L³-R³. Optionally, L³ is an organic spacer group or a fluorinated organic spacer group.

Various lithium-free electrolytes are useful with the mixtures described herein. For example, the lithium-free electrolyte optionally comprises a cross-linkable cation component and an anion component. Example cross-linkable cation components include those comprising an imidazolium group having a cross-linkable substituent. Optionally, the imidazolium group is attached to other parts of the electrolyte by organic spacer groups or fluorinated organic spacer groups. Optionally, the imidazolium group is substituted with organic groups or fluorinated organic groups. In some embodiments, the cross-linkable cation component comprises an imidazolium group with phenyl spacer groups and/or reactive cross-linking substituents.

Various anion components are useful with the lithium-free electrolytes useful with mixtures of this aspect. For example, the anion component optionally comprises

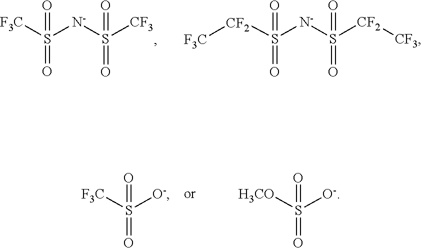

As described above, example HTS groups may exhibit properties providing utility as a hole transport structure. For example, in embodiments, HTS is an organic or heterorganic group having a band gap of between 1.4 eV and 3.5 eV, or an ionization potential of between 4.5 eV and 5.5 eV. Optionally, HTS is a monovalent group comprising one or more homocyclic, heterocyclic, aromatic, or heteroaromatic substituents that are fused or unfused. Optional heterocyclic substituents for HTS include oxygen, sulfur, selenium, tellurium, nitrogen, phosphorus, silicon, germanium, boron, aluminum, a transition metal, or a transition metal oxide. Optional aromatic or heteroaromatic substituents for HTS include a phenyl, a fused phenyl, a heterocycle, or a fused heterocycle. In some embodiments, at least one substituent of HTS comprises triarylamine, carbazole, furan, thiophene, pyridine, or combinations thereof.

Optionally, HTS is substituted with an organic group or hetero-organic group, such as an organic group or hetero-organic group having one more reactive cross-linking substituents. In a specific embodiment, HTS comprises one or more diphenyl amino groups substituted with reactive cross-linking groups. Optionally, HTS comprises a substituted carbazole. Optionally, hole transport structures useful with mixtures of this aspect comprise one or more reactive cross-linking groups. By including a reactive cross-linking group in a hole transport structure of mixtures of this aspect, the mixture may be induced to undergo cross-linking, such as between different molecules including the hole transport structures, or between molecule including the hole transport structure and a cross-linkable lithium free electrolyte.

In specific embodiments, HTS-L³-R³ has a formula of

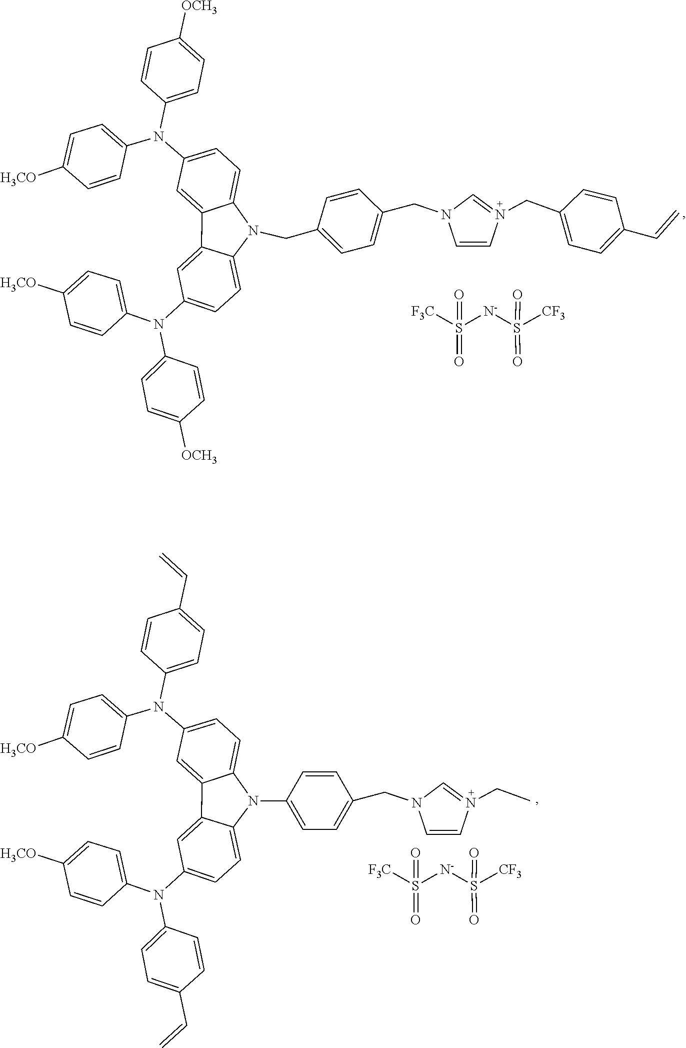

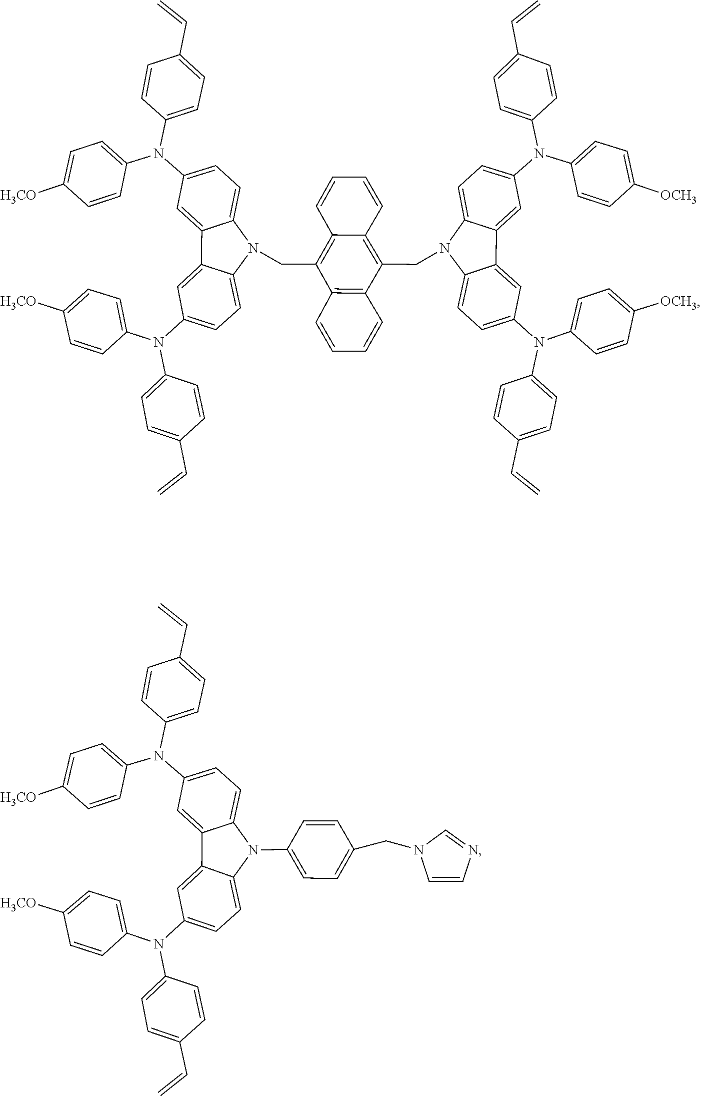 [SMILES: C=Cc1ccc(N(c2ccc(OC)cc2)c2ccc3c(c2)c2cc(N(c4ccc(C=C)cc4)c4ccc(OC)cc4)ccc2n3Cc2c3ccccc3c(Cn3c4ccc(N(c5ccc(C=C)cc5)c5ccc(OC)cc5)cc4c4cc(N(c5ccc(C=C)cc5)c5ccc(OC)cc5)ccc43)c3ccccc23)cc1 ; C=Cc1ccc(N(c2ccc(OC)cc2)c2ccc3c(c2)c2cc(N(c4ccc(C=C)cc4)c4ccc(OC)cc4)ccc2n3-c2ccc(Cn3ccnc3)cc2)cc1]

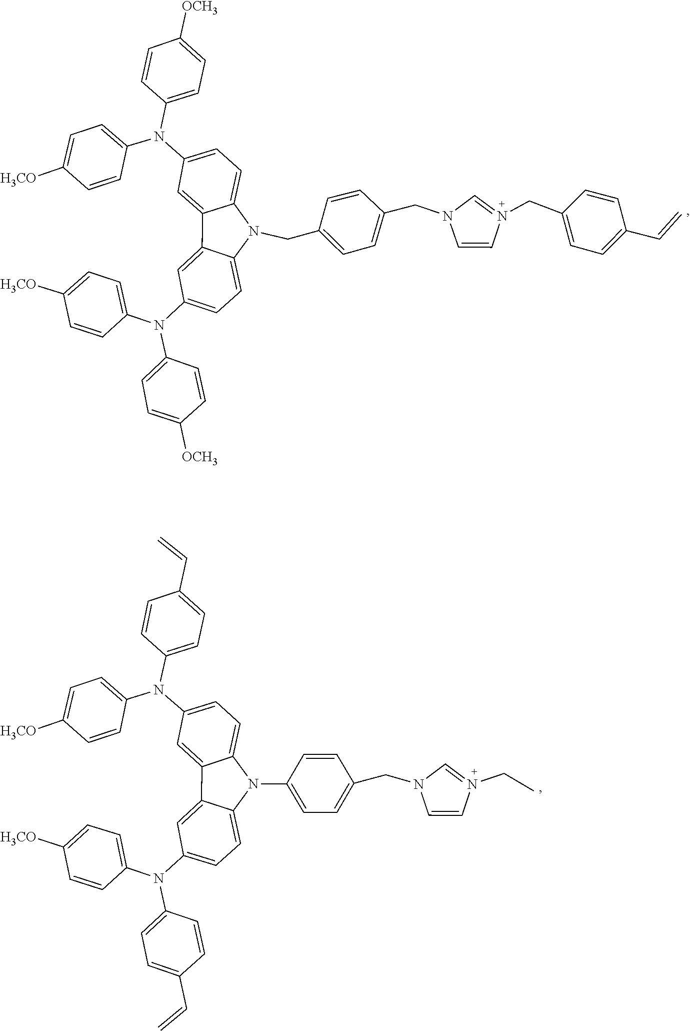

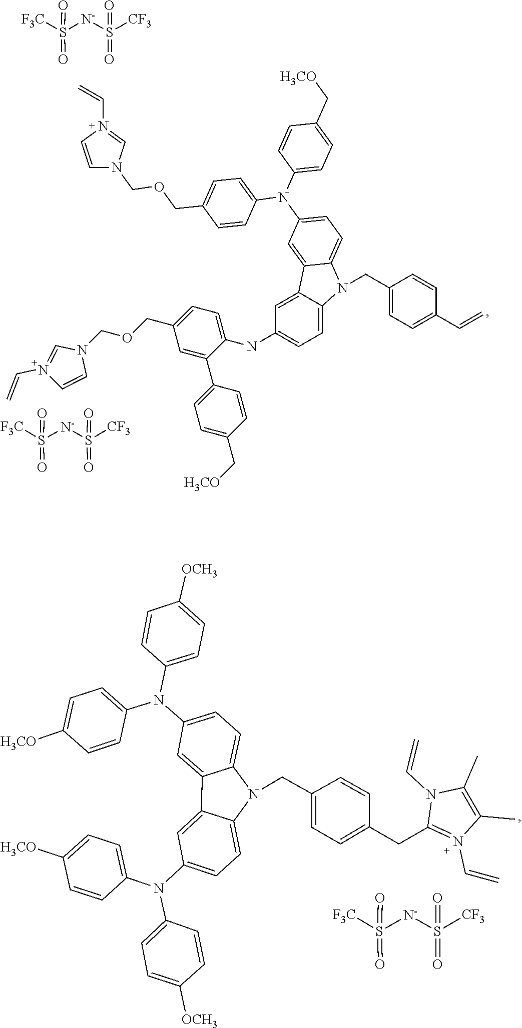

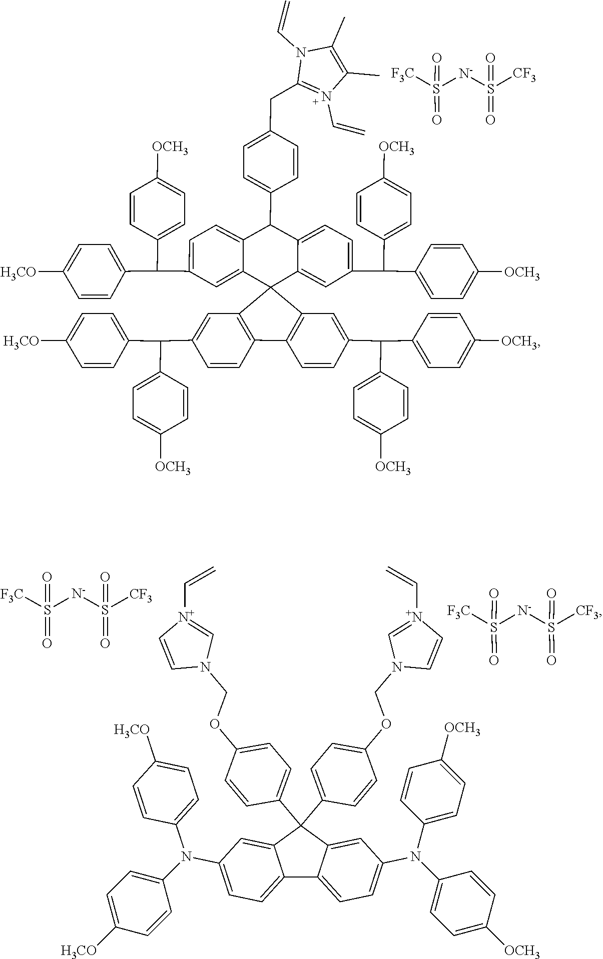

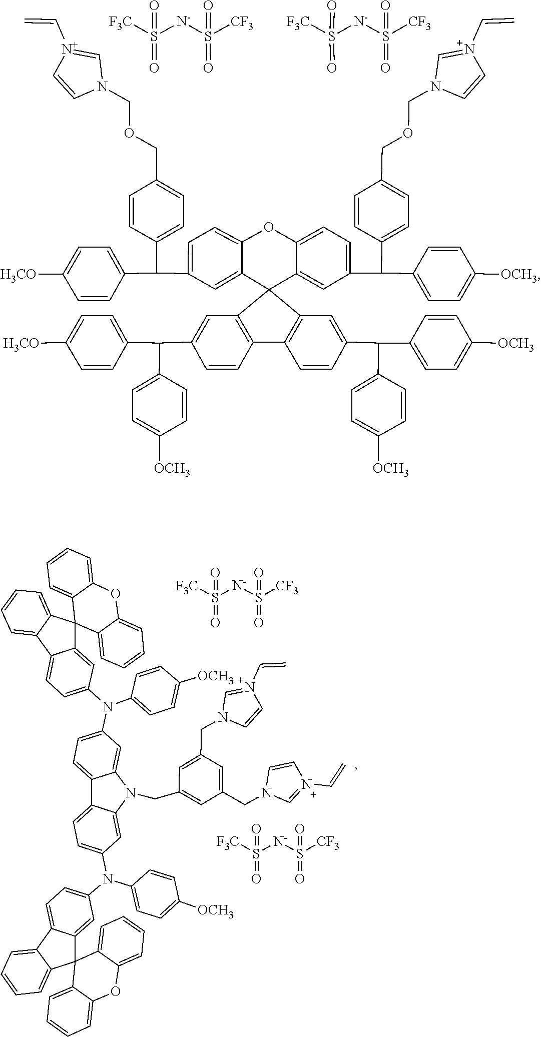

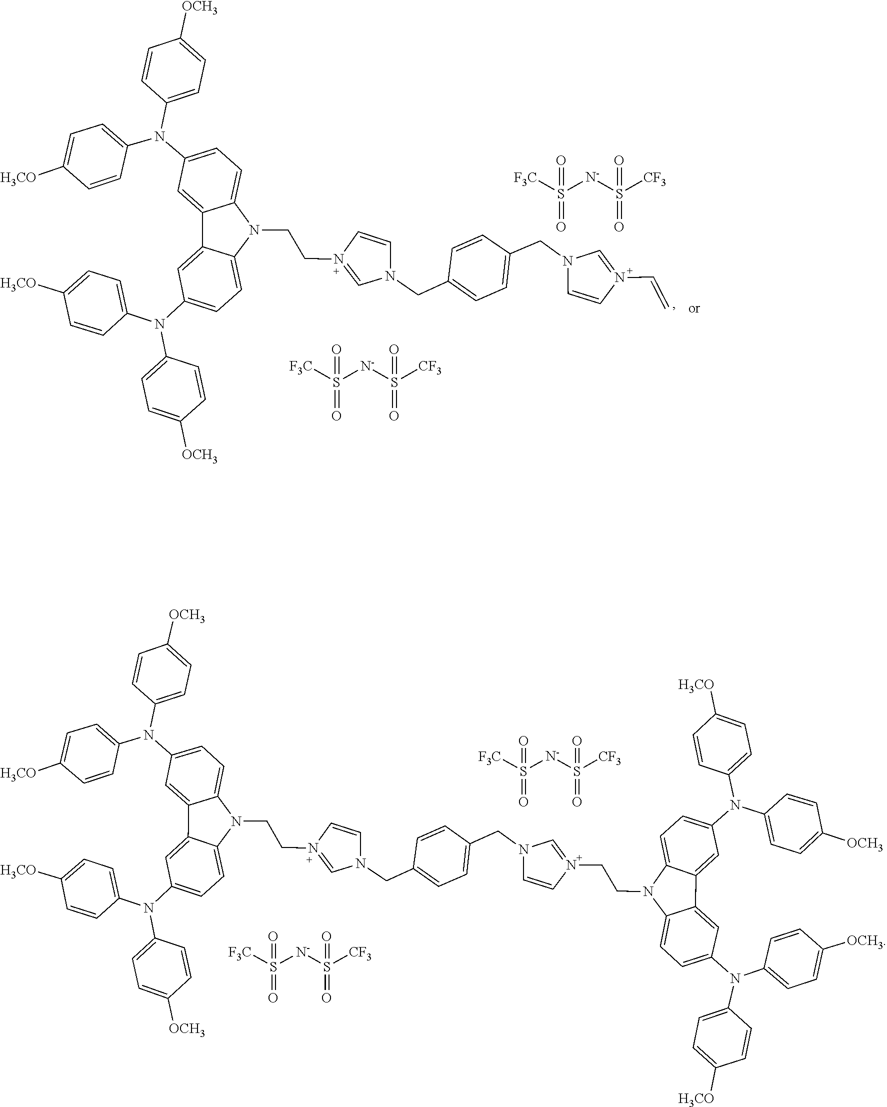

Optionally, HTS-L³-R³ comprises a hole transport compound described herein, such as a compound of the previously described aspect. Mixtures of this aspect may optionally include one or more second hole transport compounds.

In another aspect, methods of making hole transport layers are described. In embodiments, a method of this aspect comprises forming a film comprising a hole transport compound or mixture, such as a compound or mixture described herein, dissolved in a solvent. Optionally, methods of this aspect may further comprise initiating a cross-linking reaction between molecules of the hole transport compound or molecules of the mixture. For example, initiating the cross-linking reaction optionally includes heating the film or exposing the film to ultraviolet light, visible light, and/or infrared light.

In another aspect, photoactive devices are described herein. A photoactive device of some embodiments comprises: first electrode; a hole transport layer in electrical communication with the electrode, such as a hole transport layer that comprises one or more of the hole transport compounds or mixtures described herein; a photoactive layer in electrical communication with the hole transport layer; and a second electrode in electrical communication with the photoactive layer.

Various photoactive devices may be correspond to those of this aspect. Photoactive devices may correspond to photovoltaic cells, for example, or light emitting diodes. Various photoactive layer materials may be used with the photoactive devices described herein. For example, in some embodiments, the photoactive layer includes a material having a perovskite structure. Alternatively or additionally, the photoactive layer includes an organic semiconductor and or an inorganic semiconductor.

Optionally, the photoactive devices may further comprise an electron transport layer in electrical communication with the photoactive layer and the second electrode. For example, the electron transport layer may include TiO₂ or a TiO₂ containing sub-layer.

Methods of making compounds, such as hole transport compounds, are also described herein. For example, in one embodiment, a method of making a compound comprises reacting

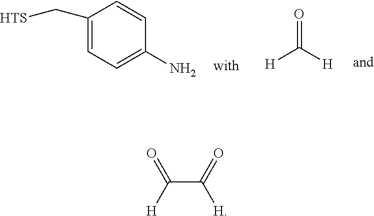

Optionally methods of this aspect may further comprise reducing

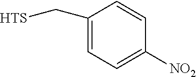

to generate

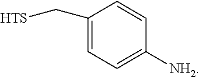

In another embodiment, a method of making a compound comprises reacting

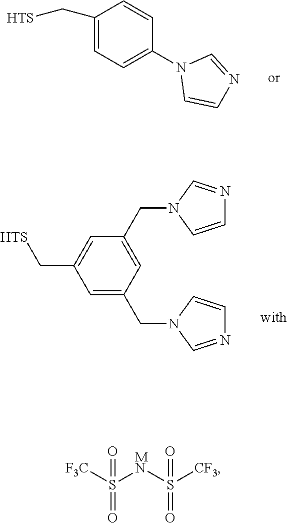

and R¹⁰—Br, where M is a metal and R¹⁰ is a C₁-C₂₀ branched or unbranched alkyl group. Optionally, methods of this aspect may further comprise reacting HTS-H or

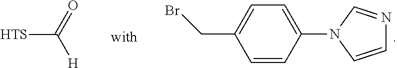

Optionally, methods of this aspect may further comprise reacting HTS-H or

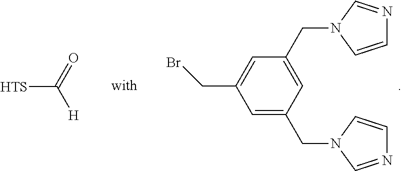

Optionally methods of this aspect may comprise or further comprise reacting

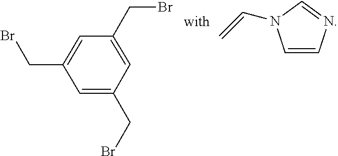

Optionally, methods of this aspect may comprise or further comprise reacting

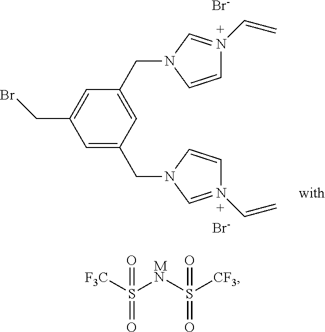

wherein M is a metal, such as lithium. Optionally, methods of this aspect may comprise or further comprise reacting

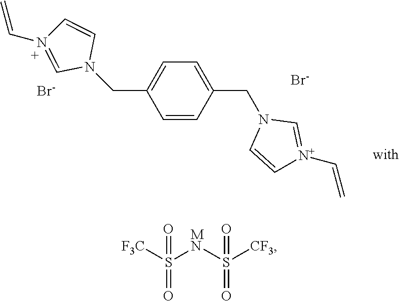

wherein M is a metal. Optionally, methods of this aspect may comprise or further comprise reacting

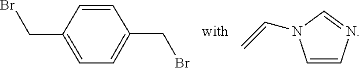

Numerous benefits are achieved by way of the present invention over conventional techniques. For example, embodiments of the present invention provide hole transport layers that degrade at a rate that is smaller than prior hole transport layers. Hole transport layers including spiro-OMeTAD, for example, may undergo phase separation, where a lithium-containing electrolyte component of the hole transport layer may separate from the component (spiro-OMeTAD) that is responsible for hole transport. As more and more phase separation occurs, performance of the hole transport layer degrades. Use of the disclosed compounds and mixtures, in both cross-linked and non-cross-linked forms, in hole transport layers provides an improvement, as these compounds and mixtures do not phase separate or only phase separate at a rate that is considerably slower than materials used in prior hole transport layers. The reduction in phase separation between the electrolyte and the hole transport component may occur because these components may be physically covalently bonded to one another. The covalently bonded structures may also exhibit a packing or stacking configuration, brought about by the planarity of the chemical structures or the charge distribution and resultant electrostatic attraction of the chemical structures. Moreover, cross-linking of the materials in the hole transport layer may further lock-in the structure or morphology of the hole transport layer, minimizing phase separation even further and optionally allowing for improved processing and operation of the photovoltaic devices

Another benefit achieved by the present invention includes the elimination or reduction of lithium in a hole transport layer. Elimination or reduction of lithium is also beneficial for reducing the rate at which degradation of a hole transport layer or a photovoltaic cell including the hole transport layer occurs. The presence of lithium may allow undesirable side reactions with oxygen (O₂) or water (H₂O) to occur within the hole transport layer. The products of these side reactions may degrade the active materials in a thin film solar cell, such as a perovskite material and reaction products occurring upon degradation may also be corrosive, further expediting the degradation of the active materials, the hole transport layer, and the electrodes.

The above and other embodiments of the invention along with many of its advantages and features are described in more detail in conjunction with the text below and attached figures.

### BRIEF DESCRIPTION OF THE DRAWINGS

FIG. 1 provides a schematic illustration of an example device incorporating a hole transport layer.

FIG. 2A, FIG. 2B, FIG. 2C, and FIG. 2D provide schematic representations of various hole transport compounds of the invention.

FIG. 3 provides a schematic illustration of a hole transport layer in non-cross-linked and cross-linked configurations.

FIG. 4 depicts a synthetic pathway for formation of a hole transport compound.

FIG. 5 depicts a synthetic pathway for formation of a hole transport compound.

FIG. 6A and FIG. 6B provides schematic representations of cross-linkable electrolytes useful with mixtures and devices described herein.

FIG. 7A, and FIG. 7B provide schematic representations of cross-linkable hole transport compounds useful with mixtures and devices described herein.

FIG. 8 provides a schematic illustration of a hole transport layer in non-cross-linked and cross-linked configurations.

FIG. 9 depicts a synthetic pathway for formation of a hole transport compound.

### DETAILED DESCRIPTION OF SPECIFIC EMBODIMENTS

Described are compounds and mixtures that may be useful in hole transport layers of photovoltaic devices, such as perovskite solar cells, or light emitting devices, such as light emitting diodes (LEDs). The compounds and mixtures include non-lithium containing or lithium-free electrolytes, such as imidazolium-based electrolytes, and small-molecule hole transport structures, such as N,N-di-p-methoxy phenyl amine-based structures. The hole transport structures and electrolytes may be covalently bonded or may be separate molecules. The hole transport structures and electrolytes may include cross-linkable groups and may be cross-linked. Devices employing the compounds and mixtures as hole transport layers are also described, such as photovoltaic devices. Synthetic methods of making small-molecule hole transport compounds are also described.

In general the terms and phrases used herein have their art-recognized meaning, which can be found by reference to standard texts, journal references and contexts known to those skilled in the art. The following definitions are provided to clarify their specific use in the context of the invention.

“Hole transport compound” refers to a molecule that permits transmission of a hole (i.e., an absence of an electron) from a first nearby molecule or material (e.g., an electrolyte, dopant, another hole transport compound, an electrode, or a current collector) to second nearby molecule or material. Stated another way, a hole transport compound can be oxidized by providing an electron to the first nearby molecule or material (equivalent to accepting a hole from the first nearby molecule or material) and then can be reduced by accepting an electron from the second nearby molecule or material (equivalent to providing a hole to the second nearby molecule or material). A “hole transport structure” refers to a group, moiety, or other portion of a molecule that is responsible for providing transmission of the hole/electron between nearby molecules or materials. A hole transport layer may correspond to a bulk layer made up of many hole transport compound molecules and allow for propagation of a hole/electron between different bulk structures, such as between a photoactive layer and an electrode, for example. Within the hole transport layer, different hole transport compounds may pass the hole/electrons between one another to enable the overall transmission of holes/electrons between the bulk structures.

“Electrolyte” refers to an ionic compound having an anion component and a cation component and that dissociates into separate cation and anion components when dissolved in a solvent, such as a polar solvent, with each of the ionic components solvated by molecules of the solvent. Electrolytes may also be referred to as “salts.” In some embodiments, electrolytes useful herein include those comprising ionic liquids or ionic solids. Example electrolytes described herein include those in which a cation component includes an imidazolium core structure that carries a positive charge. Electrolytes may function as dopants and/or redox mediators in a hole transport layer or electron transport layer to increase conductivity of holes or electrons through the layer. As an example, an electrolyte may correspond to a p-type dopant having a lowest-unoccupied molecular orbital that is aligned in energy with a highest occupied molecular orbital of a hole transport structure.

“Cross-linkable” or “crosslinkable” refers to the ability of a reactive group to form covalent bonds (i.e., cross-link) with another, appropriately structured, reactive groups. In some embodiments, cross-linking (or crosslinking) may take place only via introduction of energy to drive the cross-linking reaction that may involve bond-forming and optionally bond-breaking. Such energy may be provided in the form of electromagnetic radiation (e.g., ultraviolet light, visible light, infrared light) or heat. In some embodiments, cross-linking may take place by bringing appropriately reactive cross-linkable structures into close proximity. Useful cross-linkable groups include, but are not limited to —NH₂, —OH, —SH, —SiCl₃, —Si(OH)₃,

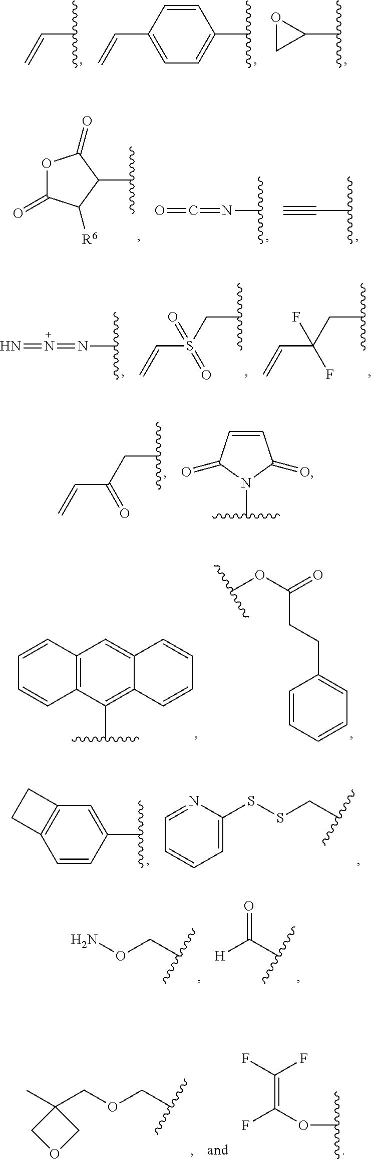

Example cross-linking reactions include, but are not limited to, vinyl cross-linking reactions, styrenic cross-linking reactions, epoxy-based cross-linking reactions, urethane-based cross-linking reactions, isocyanate-based cross-linking reactions, copper/click-based cross-linking reactions, siloxane-based cross-linking reactions, oxo-Michael-based cross-linking reactions, aza-Michael-based cross-linking reactions, thio-Michael-based cross-linking reactions, Diels-Alder-based cross-linking (cycloaddition) reactions, cinnamic-based cross-linking (cycloaddition) reactions, cyclobutane-based cross-linking (cycloaddition) reactions, thiol-ene-based crosslinking reactions, pyridyl disulfide-based cross-linking reactions, oxime-based cross-linking reactions, oxetane-based cross-linking reactions, and perfluorocyclobutane-based cross-linking reactions. Optionally, a reactive cross-linking substituent (i.e., a cross-linkable group) comprises a hydrocarbon. Optionally, a reactive cross-linking substituent is a vinyl group or a styrene group.

In an embodiment, disclosed compositions or compounds are isolated or purified. In an embodiment, an isolated or purified compound is at least partially isolated or purified as would be understood in the art. In an embodiment, a disclosed composition or compound has a chemical purity of 90%, optionally for some applications 95%, optionally for some applications 99%, optionally for some applications 99.9%, optionally for some applications 99.99%, and optionally for some applications 99.999% pure.

Many of the molecules disclosed herein contain one or more ionizable groups. Ionizable groups include groups from which a proton can be removed (e.g., —COOH) or added (e.g., amines) and groups which can be quaternized (e.g., amines). All possible ionic forms of such molecules and salts thereof are intended to be included individually in the disclosure herein. With regard to salts of the compounds described herein, it will be appreciated that a wide variety of available counter-ions may be selected that are appropriate for preparation of salts of this invention for a given application. In specific applications, the selection of a given anion or cation for preparation of a salt can result in increased or decreased solubility of that salt.

The disclosed compounds optionally contain one or more chiral centers. Accordingly, this disclosure includes racemic mixtures, diasteromers, enantiomers, tautomers and mixtures enriched in one or more stereoisomer. Disclosed compounds including chiral centers encompass the racemic forms of the compound as well as the individual enantiomers and non-racemic mixtures thereof.

As used herein, the terms “group” and “moiety” may refer to a functional group of a chemical compound. Groups of the disclosed compounds refer to an atom or a collection of atoms that are a part of the compound. Groups of the disclosed compounds may be attached to other atoms of the compound via one or more covalent bonds. Groups may also be characterized with respect to their valence state. The present disclosure includes groups characterized as monovalent, divalent, trivalent, etc. valence states. In embodiments, the term “substituent” may be used interchangeably with the terms “group” and “moiety.”

As is customary and well known in the art, hydrogen atoms in chemical formulas disclosed herein are not always explicitly shown, for example, hydrogen atoms bonded to the carbon atoms of aliphatic, aromatic, alicyclic, carbocyclic and/or heterocyclic rings are not always explicitly shown in the formulas recited. The structures provided herein, for example in the context of the description of any specific formulas and structures recited, are intended to convey the chemical composition of disclosed compounds of methods and compositions. It will be appreciated that the structures provided do not indicate the specific positions of atoms and bond angles between atoms of these compounds.

As used herein, the terms “alkylene” and “alkylene group” are used synonymously and refer to a divalent group derived from an alkyl group as defined herein. The present disclosure includes compounds having one or more alkylene groups. Alkylene groups in some compounds function as attaching and/or spacer groups. Disclosed compounds optionally include substituted and/or unsubstituted C₁-C₂₀ alkylene, C₁-C₁₀ alkylene and C₁-C₅ alkylene groups.

As used herein, the terms “cycloalkylene” and “cycloalkylene group” are used synonymously and refer to a divalent group derived from a cycloalkyl group as defined herein. The present disclosure includes compounds having one or more cycloalkylene groups. Cycloalkyl groups in some compounds function as attaching and/or spacer groups. Disclosed compounds optionally include substituted and/or unsubstituted C₃-C₂₀ cycloalkylene, C₃-C₁₀ cycloalkylene and C₃-C₅ cycloalkylene groups.

As used herein, the terms “arylene” and “arylene group” are used synonymously and refer to a divalent group derived from an aryl group as defined herein. The present disclosure includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as attaching and/or spacer groups. Arylene groups in some compounds function as chromophore, fluorophore, aromatic antenna, dye and/or imaging groups. Disclosed compounds optionally include substituted and/or unsubstituted C₃-C₃₀ arylene, C₃-C₂₀ arylene, C₃-C₁₀ arylene and C₁-C₅ arylene groups.

As used herein, the terms “heteroarylene” and “heteroarylene group” are used synonymously and refer to a divalent group derived from a heteroaryl group as defined herein. The present disclosure includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as attaching and/or spacer groups. Heteroarylene groups in some compounds function as chromophore, aromatic antenna, fluorophore, dye and/or imaging groups. Disclosed compounds optionally include substituted and/or unsubstituted C₃-C₃₀ heteroarylene, C₃-C₂₀ heteroarylene, C₁-C₁₀ heteroarylene and C₃-C₅ heteroarylene groups.

As used herein, the terms “alkenylene” and “alkenylene group” are used synonymously and refer to a divalent group derived from an alkenyl group as defined herein. The present disclosure includes compounds having one or more alkenylene groups. Alkenylene groups in some compounds function as attaching and/or spacer groups. Disclosed compounds optionally include substituted and/or unsubstituted C₂-C₂₀ alkenylene, C₂-C₁₀ alkenylene and C₂-C₅ alkenylene groups.

As used herein, the terms “cylcoalkenylene” and “cylcoalkenylene group” are used synonymously and refer to a divalent group derived from a cylcoalkenyl group as defined herein. The present disclosure includes compounds having one or more cylcoalkenylene groups. Cycloalkenylene groups in some compounds function as attaching and/or spacer groups. Disclosed compounds optionally include substituted and/or unsubstituted C₃-C₂₀ cylcoalkenylene, C₃-C₁₀ cylcoalkenylene and C₃-C₅ cylcoalkenylene groups.

As used herein, the terms “alkynylene” and “alkynylene group” are used synonymously and refer to a divalent group derived from an alkynyl group as defined herein. The present disclosure includes compounds having one or more alkynylene groups. Alkynylene groups in some compounds function as attaching and/or spacer groups. Disclosed compounds optionally include substituted and/or unsubstituted C₂-C₂₀ alkynylene, C₂-C₁₀ alkynylene and C₂-C₅ alkynylene groups.

As used herein, the term “halo” refers to a halogen group, such as a fluoro (—F), chloro (—Cl), bromo (—Br), or iodo (—I).

The term “heterocyclic” refers to ring structures containing at least one other kind of atom, in addition to carbon, in the ring. Examples of such atoms include sulfur, selenium, tellurium, nitrogen, phosphorus, silicon, germanium, boron, aluminum, and a transition metal. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic” refers to a ring that is not an aromatic ring. Alicyclic rings include both carbocyclic and heterocyclic rings.

The term “aliphatic” refers to non-aromatic hydrocarbon compounds and groups. Aliphatic groups generally include carbon atoms covalently bonded to one or more other atoms, such as carbon and hydrogen atoms. Aliphatic groups may, however, include a non-carbon atom, such as an oxygen atom, a nitrogen atom, a sulfur atom, etc., in place of a carbon atom. Non-substituted aliphatic groups include only hydrogen substituents. Substituted aliphatic groups include non-hydrogen substituents, such as halo groups and other substituents described herein. Aliphatic groups can be straight chain, branched, or cyclic. Aliphatic groups can be saturated, meaning only single bonds join adjacent carbon (or other) atoms. Aliphatic groups can be unsaturated, meaning one or more double bonds or triple bonds join adjacent carbon (or other) atoms.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alkyl group having a ring structure such as ring structure comprising 3-30 carbon atoms, optionally 3-20 carbon atoms and optionally 3-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted.

Substituted alkyl groups include, among others, those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully-halogenated or semi-halogenated alkyl groups, such as alkyl groups having one or more hydrogens replaced with one or more fluorine atoms, chlorine atoms, bromine atoms and/or iodine atoms. Substituted alkyl groups include fully-fluorinated or semi-fluorinated alkyl groups, such as alkyl groups having one or more hydrogens replaced with one or more fluorine atoms.

An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alkyl portion of the groups is substituted as provided herein in connection with the description of alkyl groups. As used herein MeO— refers to CH₃O—.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 4 carbon atoms. Alkenyl groups include medium length alkenyl groups having from 5-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully-halogenated or semi-halogenated alkenyl groups, such as alkenyl groups having one or more hydrogens replaced with one or more fluorine atoms, chlorine atoms, bromine atoms and/or iodine atoms. Substituted alkenyl groups include fully-fluorinated or semi-fluorinated alkenyl groups, such as alkenyl groups having one or more hydrogen atoms replaced with one or more fluorine atoms.

Aryl groups include groups having one or more 5-, 6- or 7-member aromatic and/or heterocyclic aromatic rings. The term heteroaryl specifically refers to aryl groups having at least one 5-, 6- or 7-member heterocyclic aromatic rings. Aryl groups can contain one or more fused aromatic and heteroaromatic rings or a combination of one or more aromatic or heteroaromatic rings and one or more non-aromatic rings that may be fused or linked via covalent bonds. Heterocyclic aromatic rings can include one or more N, O, or S atoms in the ring, among others. Heterocyclic aromatic rings can include those with one, two or three N atoms, those with one or two O atoms, and those with one or two S atoms, or combinations of one or two or three N, O or S atoms, among others. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

Arylalkyl and alkylaryl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl and arylalkyl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully-halogenated or semi-halogenated arylalkyl groups, such as arylalkyl groups having one or more alkyl and/or aryl groups having one or more hydrogens replaced with one or more fluorine atoms, chlorine atoms, bromine atoms and/or iodine atoms.

As to any of the groups described herein which contain one or more substituents, it is understood that such groups do not contain any substitution or substitution patterns which are sterically impractical and/or synthetically non-feasible. In addition, the disclosed compounds include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others:

halogen, including fluorine, chlorine, bromine or iodine;
pseudohalides, including —CN;
—COOR where R is a hydrogen or an alkyl group or an aryl group or, more specifically, where R is a methyl, ethyl, propyl, butyl, or phenyl group, all of which are optionally substituted;
—COR where R is a hydrogen or an alkyl group or an aryl group or, more specifically, where R is a methyl, ethyl, propyl, butyl, or phenyl group, all of which are optionally substituted;
—CON(R)₂ where each R, independently of each other R, is a hydrogen or an alkyl group or an aryl group or, more specifically, where R is a methyl, ethyl, propyl, butyl, or phenyl group, all of which are optionally substituted; and where R and R can form a ring which can contain one or more double bonds and can contain one or more additional carbon atoms;
—OCON(R)₂ where each R, independently of each other R, is a hydrogen or an alkyl group or an aryl group and, more specifically, where R is a methyl, ethyl, propyl, butyl, or phenyl group, all of which are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group or, more specifically, where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group or, more specifically, where R is hydrogen, methyl, ethyl, propyl, butyl, or a phenyl group, all of which are optionally substituted;
—SO₂R, or —SOR where R is an alkyl group or an aryl group or, more specifically, where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, an alkyl group, or an aryl group, all of which are optionally substituted, and wherein R and R can form a ring which can contain one or more double bonds and can contain one or more additional carbon atoms; or
—OR where R is H, an alkyl group, an aryl group, or an acyl group, all of which are optionally substituted. In a particular example R can be an acyl, yielding —OCOR″ where R″ is a hydrogen or an alkyl group or an aryl group and more specifically where R″ is methyl, ethyl, propyl, butyl, or phenyl groups, all of which are optionally substituted.

Specific substituted alkyl groups include haloalkyl groups, particularly trihalomethyl groups and specifically trifluoromethyl groups. Specific substituted aryl groups include mono-, di-, tri, tetra- and penta-halo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

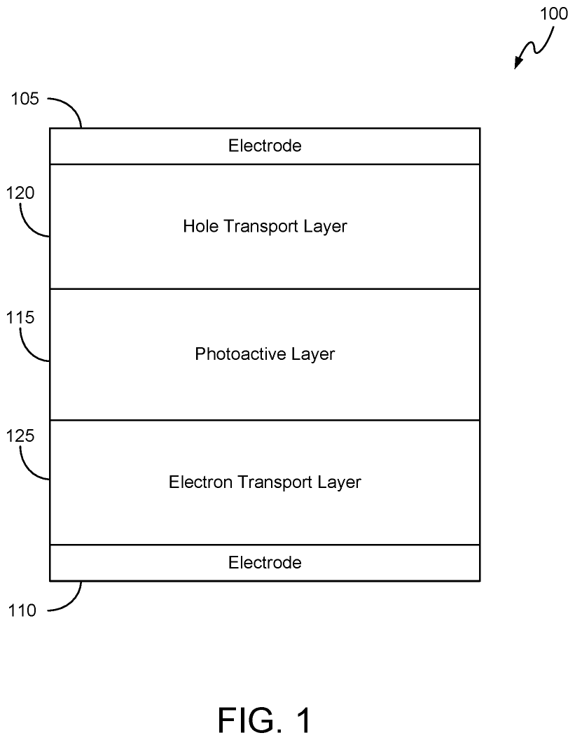

FIG. 1 provides a schematic illustration of an example device 100. Device 100 includes electrodes 105 and 110. Electrodes 105 and 110 may comprise a metal (e.g., aluminum, copper, silver, gold, etc.) or a transparent electrode, such as a transparent conducting oxide (e.g., indium tin oxide or fluorine doped tin oxide). Use of a transparent electrode is advantageous for allowing incident electromagnetic radiation to pass through the electrode and reach other underlying components in the photovoltaic device or for allowing electromagnetic radiation generated within the photoactive layer to be emitted through the electrode.

Device 100 also includes a photoactive layer 115. In embodiments where device 100 is a photovoltaic device, photoactive layer 115 may correspond to a semiconducting material that absorbs photons possessing energy equal to or greater than a band gap of the semiconducting material to generate an electron-hole pair and an associated voltage and current. Example photoactive materials include, but are not limited to, perovskite structured compounds, such as a methylammonium lead halide (e.g., methylammonium lead iodide) compound.

In other embodiments, device 100 is a light emitting device, such as a light emitting diode. Here, photoactive layer 115 may correspond to a semiconducting material that generates photons possessing when electron-hole pairs are recombined within the semiconducting material, such as at a P-N junction. Example photoactive materials include, but are not limited to, inorganic semiconductors, such as combinations of one or more of gallium, arsenic, aluminum, nitrogen, phosphorus, indium, zinc, selenium, silicon, carbon, and boron, and organic semiconductors, such as organometallic chelates, fluorescent and phosphorescent dyes, conjugated dendrimers, electroluminescent conductive polymers, and phosphorescent organic materials.

Device 100 also includes a hole transport layer 120. Hole transport layer 120 may comprise hole transport compounds as described herein or a mixture of a hole transport compound and an electrolyte, as described herein. Optionally, the hole transport layer comprises a solvent, such as a polar solvent. In embodiments, the hole transport layer is formed by forming a film comprising a hole transport compound dissolved in a solvent, such as on a surface of an electrode or on a surface of a photoactive layer. Optionally, the hole transport layer is formed as a film that is transferred to an interface between an electrode and a photoactive layer. Optionally, the hole transport compound is mixed with an electrolyte and this mixture is dissolved in the solvent for forming the thin film. In some embodiments, the hole transport compound is fused to (i.e., covalently bonded) to a cation component of an electrolyte. As described below, in some embodiments, the hole transport compound and/or electrolyte undergoes cross-linking. Optionally, cross-linking may be initiated by exposing the film to ultraviolet light, visible light, and/or infrared light. Optionally cross-linking may be initiated by heating the film.

Device 100 also includes an electron transport layer 125. It will be appreciated that some devices may not include an electron transport layer; thus electron transport layer 125 is an optional feature. Electron transport layer 125 may correspond to a material or structure that allows electrons from photoactive layer 115 to be propagated to electrode 110.

Optionally, electron transport layer 125 may be mixed or co-located within or overlapping the photoactive layer. Example electron transport layers include those comprising titanium dioxide.

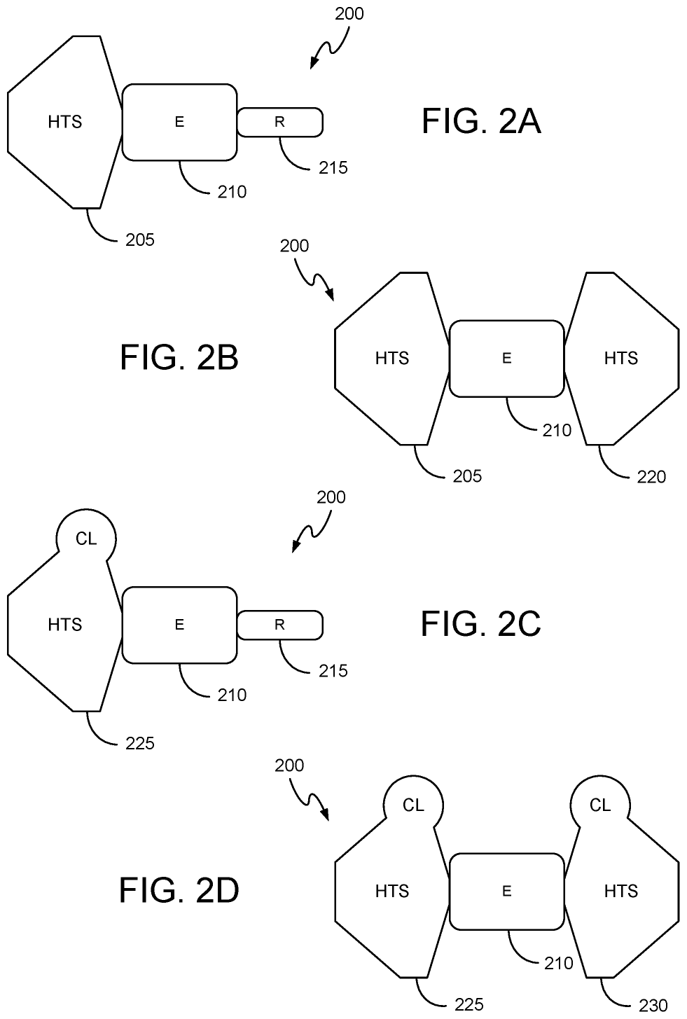

FIG. 2A, FIG. 2B, FIG. 2C, and FIG. 2D provide schematic representations of different hole transport compounds, showing different groups present within the hole transport compound. In FIG. 2A, hole transport compound 200 includes a hole transport structure (HTS) 205, an electrolyte (E) 210 and a side group (R) 215. It will be appreciated that HTS 205 and side group 215 may each correspond to monovalent groups, while electrolyte 210 may correspond to or include a bivalent group.

Example side groups 215 include, but are not limited to, a hydrogen atom, H; a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused. More particularly, side group 215 may be a branched, unbranched, cyclic, or polycyclic alkyl group that is substituted or unsubstituted; or a branched, unbranched, cyclic, or polycyclic alkenyl group that is substituted or unsubstituted; or a branched, unbranched, cyclic, or polycyclic alkynyl group that is substituted or unsubstituted; or a substituted or unsubstituted aryl group; or a substituted or unsubstituted heteroaryl group. More particularly, side group 215 may be a branched or unbranched, substituted or unsubstituted fluoroalkyl group; or a branched or unbranched, substituted or unsubstituted perfluoroalkyl group; or a branched or unbranched, substituted or unsubstituted fluoroalkenyl group; or a branched or unbranched, substituted or unsubstituted fluoroalkyne group; or a substituted or unsubstituted perfluoroaromatic or perfluoroheteroaromatic group. Optionally, side group 215 is a reactive cross-linking group. In specific embodiments, side group 215 is a methyl group, an ethyl group, or a styrene group.

Example electrolytes 210 include lithium-free electrolytes. For example a lithium-free electrolyte may include a non-lithium-containing a cation component covalently bonded to HTS 205 and side group 215 and an anion component. Useful non-lithium-containing cation components include imidazolium-based cation structures. For example, a cation of electrolyte 210 may comprise

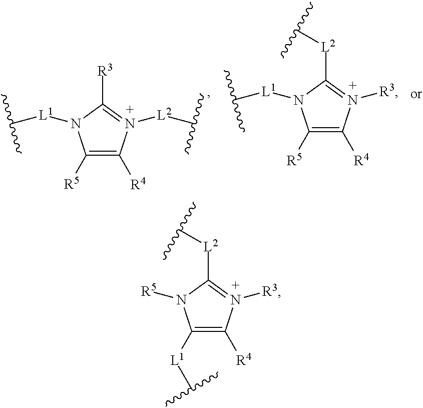

wherein L¹ and L² are independently a spacer or linking group substituent selected from the group including a C₁-C₂₀ branched, unbranched, cyclic, or polycyclic bivalent aliphatic group that is saturated or unsaturated and that is substituted or unsubstituted; and a substituted or unsubstituted bivalent aromatic group that is fused or unfused, and wherein R³, R⁴, and R⁵ are independently a reactive cross-linking group; or H; or a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused. More particularly, L¹ and L² may independently be a branched, unbranched, cyclic, or polycyclic alkylene group that is substituted or unsubstituted; or a branched, unbranched, cyclic, or polycyclic alkenylene group that is substituted or unsubstituted; or a branched, unbranched, cyclic, or polycyclic alkynylene group that is substituted or unsubstituted; or a substituted or unsubstituted arylene group; or a substituted or unsubstituted heteroarylene group. More particularly, L¹ and L² may independently be a branched or unbranched, substituted or unsubstituted fluoroalkylene group; a branched or unbranched, substituted or unsubstituted perfluoroalkyene group; a branched or unbranched, substituted or unsubstituted fluoroalkenylene group; or a substituted or unsubstituted perfluoroaromatic or perfluoroheteroaromatic group. Optionally, L¹ may be or comprise

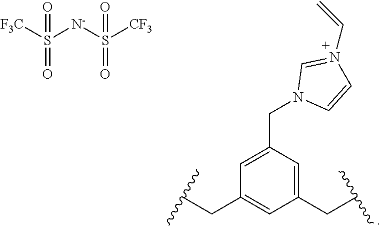

Optionally, L¹ may be or comprise

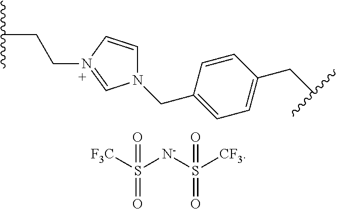

Optionally, R³, R⁴, and R⁵ are independently a branched, unbranched, cyclic, or polycyclic alkyl group that is substituted or unsubstituted; or a branched, unbranched, cyclic, or polycyclic alkenyl group that is substituted or unsubstituted; or a branched, unbranched, cyclic, or polycyclic alkynyl group that is substituted or unsubstituted; or a substituted or unsubstituted aryl group; or a substituted or unsubstituted heteroaryl group. Optionally, R³, R⁴, and R⁵ are independently a branched or unbranched, substituted or unsubstituted fluoroalkyl group; or a branched or unbranched, substituted or unsubstituted perfluoroalkyl group; or a branched or unbranched, substituted or unsubstituted fluoroalkenyl group; or a branched or unbranched, substituted or unsubstituted fluoroalkyne group; or a substituted or unsubstituted perfluoroaromatic or perfluoroheteroaromatic group.

Example reactive cross-linking groups include —NH₂, —OH, —SH, —SiCl₃, —Si(OH)₃,

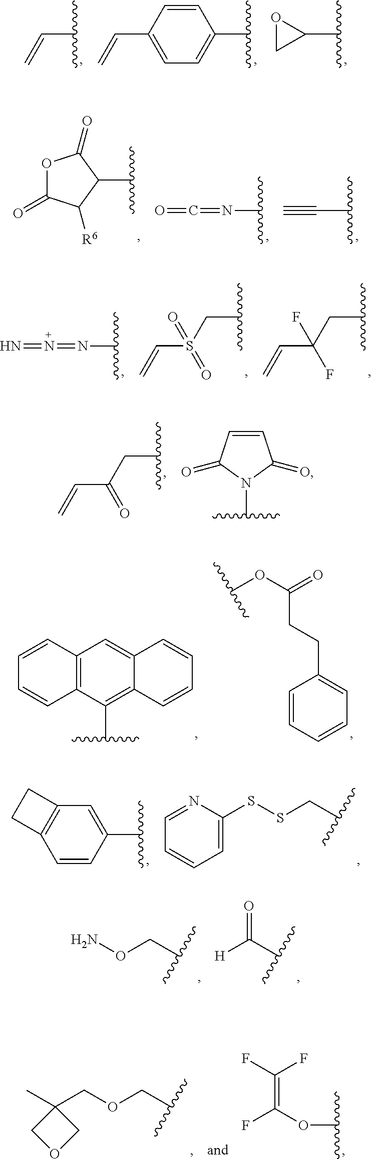

where R is independently a C₁-C₂₀ branched, unbranched, cyclic, or polycyclic monovalent aliphatic group that is saturated or unsaturated and substituted or unsubstituted, or a substituted or unsubstituted monovalent aromatic group.

Specific cation components useful for electrolyte 210 include

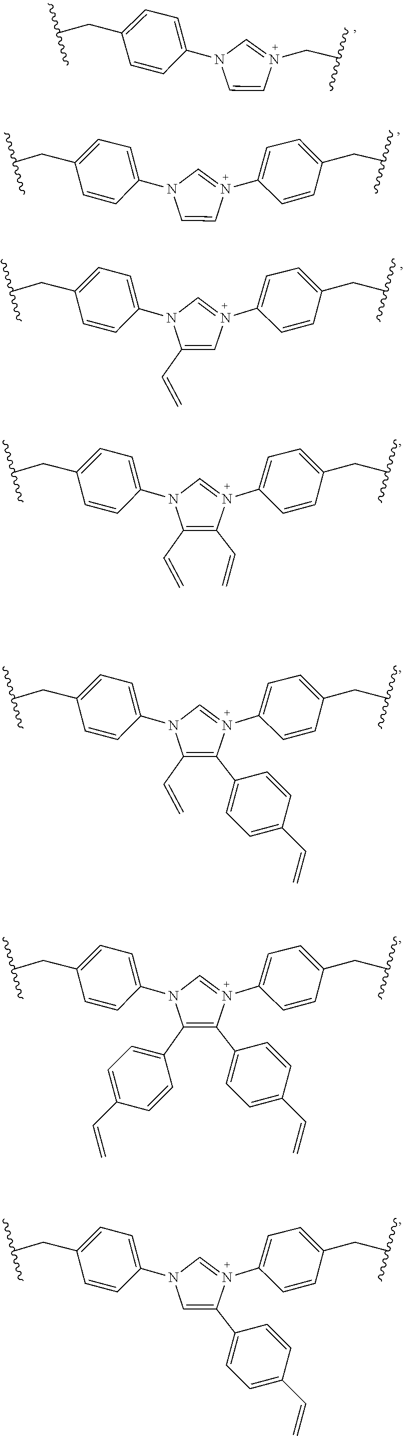

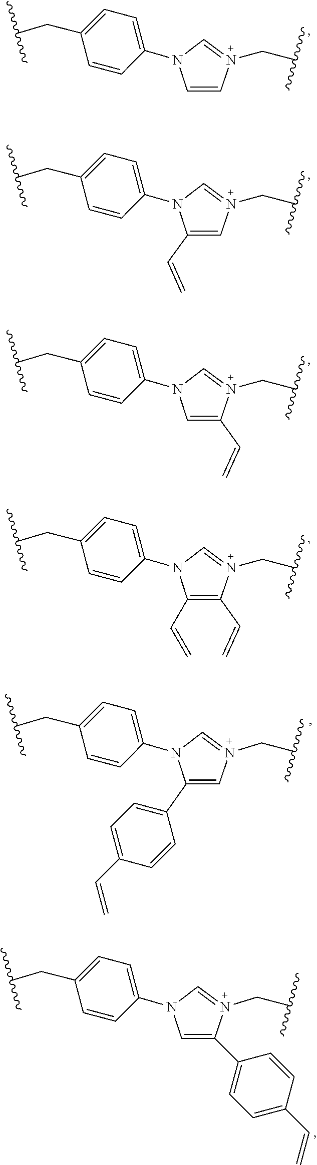

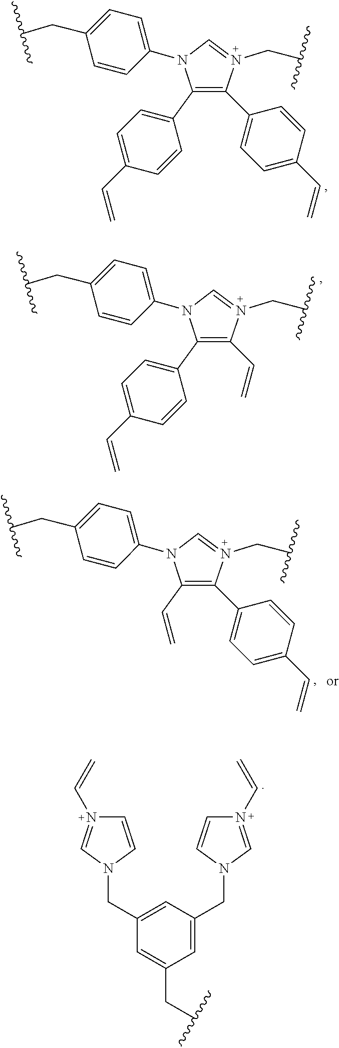

Taken together, electrolyte 210 and side group 215 may comprise, for example,

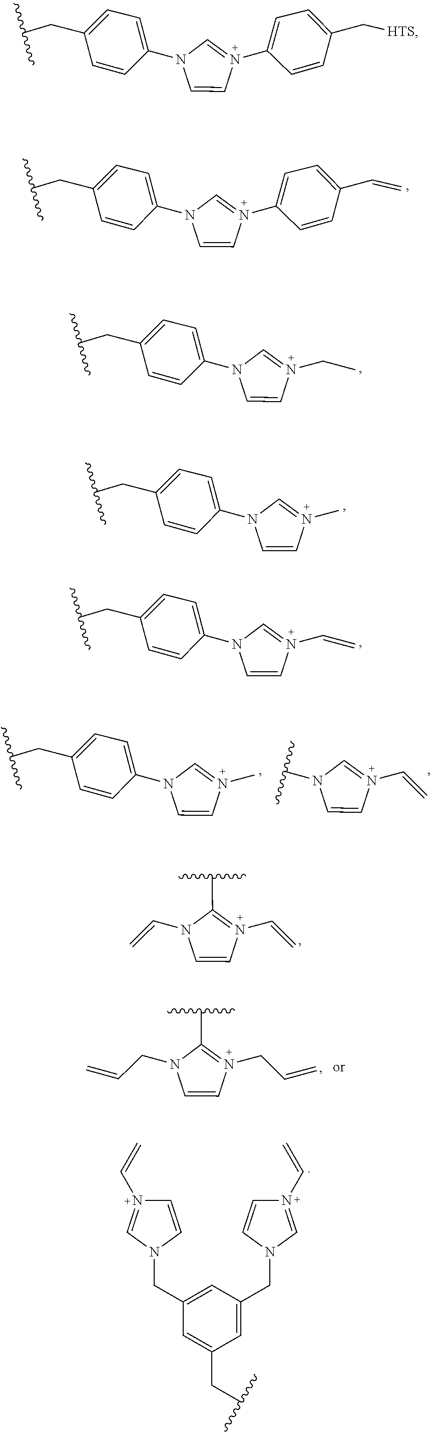

A variety of anion components are useful with electrolyte 210. For example, the anion component may comprise

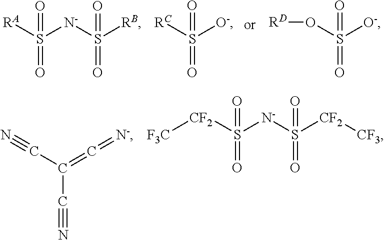

PF₆⁻, BF₄⁻, [SCN]⁻, dimethyl phosphate, [Co(SCN)₄]²⁻, [(C₂F₅)₃PF₃]⁻, or [B(CN)₄]⁻, wherein R^{A}, R^{B}, R^{C}, and R^{D} are independently H, a C₁-C₂₀ alkyl group, a C₁-C₂₀ fluorinated alkyl group, or a C₁-C₂₀ perfluorinated alkyl group. Specific anion components include, but are not limited to,

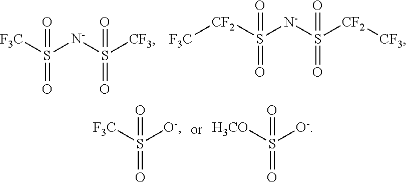

It will be appreciated that the anion component is ionically bound to the cation component. Optionally, the anion component may be covalently bonded to R¹ and HTS, while the cation component may be ionically bound to the anion component.

Optionally, side group 215 comprises another HTS. For example, FIG. 2B depicts the hole transport compound 200 in which the side group (R) 215 is another hole transport structure (HTS) 220.

Example hole transport structures 205 or 220 include, but are not limited to, organic or heterorganic groups exhibiting a band gap of between 1.4 eV and 3.5 eV and/or an ionization potential of between 4.5 eV and 5.5 eV. Such electronic properties are useful for allowing the hole transport structures 205 or 220 to provide or receive electrons/holes from a photoactive material, such as a perovskite photoactive layer. Such a band gap may correspond to an energy difference between a highest occupied molecular orbital of the hole transport structure and a lowest unoccupied molecular orbital of the hole transport structure, which may allow for favorable electron/hole transfer between the hole transport structure and the photoactive material. Useful hole transport structures include those comprising one or more homocyclic, heterocyclic, aromatic, or heteroaromatic substituents that are fused or unfused. Example heterocyclic substituents may comprise one or more of oxygen, sulfur, selenium, tellurium, nitrogen, phosphorus, silicon, germanium, boron, aluminum, a transition metal, or a transition metal oxide. Specific example aromatic or heteroaromatic substituents comprise a phenyl group, a fused phenyl group, a heterocycle, or a fused heterocycle. Optionally, the hole transport structure comprises a non-aromatic heterocycle fused to an aromatic group. Example components for HTS 205 or 220 include a triarylamine, a carbazole, a furan, a thiophene, a pyridine, or combinations of these.

Aromatic, heteroaromatic, or amine groups of a hole transport structures may be functionalized by various substituents. For example, HTS 205 or 220 may include one or more substituents selected from the group including

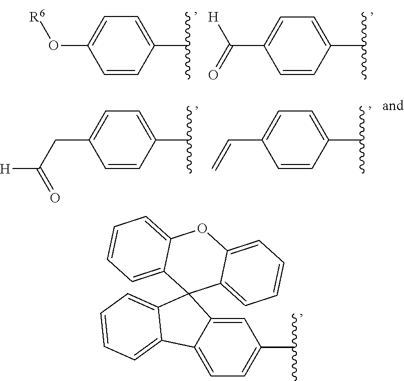

wherein each R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group. Optionally, each R⁶ is a branched, unbranched, cyclic, or polycyclic alkyl group that is substituted or unsubstituted; a branched, unbranched, cyclic, or polycyclic alkenyl group that is substituted or unsubstituted; a branched, unbranched, cyclic, or polycyclic alkynyl group that is substituted or unsubstituted; a substituted or unsubstituted aryl group; or a substituted or unsubstituted heteroaryl group.

Optionally, HTS 205 or 220 may be cross-linkable or include one or more reactive cross-linkable substituents. It will be appreciated that cross-linkable groups on different cross-linkable hole transport structures may be reactive with one another or induced to react with one another upon exposure to a sufficient energy source (such as heat, ultraviolet light, infrared light, visible light). FIG. 2C and FIG. 2D depict schematic representations of a hole transport compound where the hole transport structure 225 or 230 is cross-linkable (CL) or includes a cross-linkable group. For example, HTS 225 or 230 may include one or more substituents selected from the group including

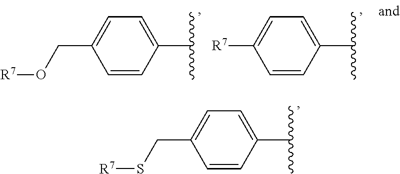

wherein each R⁷ is independently a reactive cross-linkable substituent. In specific examples, R⁷ is selected from the group including:

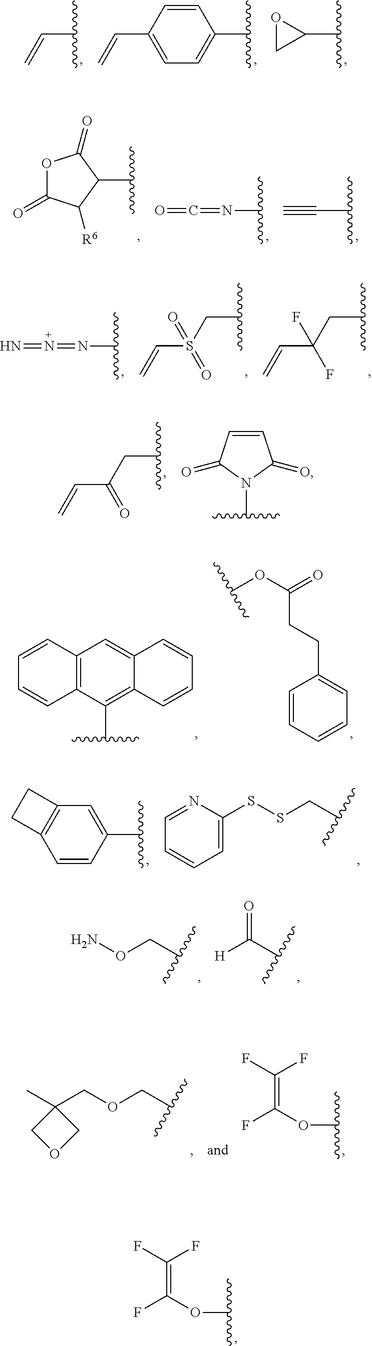

wherein R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group. Optionally, R⁶ is a branched, unbranched, cyclic, or polycyclic alkyl group that is substituted or unsubstituted; a branched, unbranched, cyclic, or polycyclic alkenyl group that is substituted or unsubstituted; a branched, unbranched, cyclic, or polycyclic alkynyl group that is substituted or unsubstituted; a substituted or unsubstituted aryl group; or a substituted or unsubstituted heteroaryl group.

Example hole transport structures 205, 220, 225, or 230 may include one or more substituents independently selected from the group including:

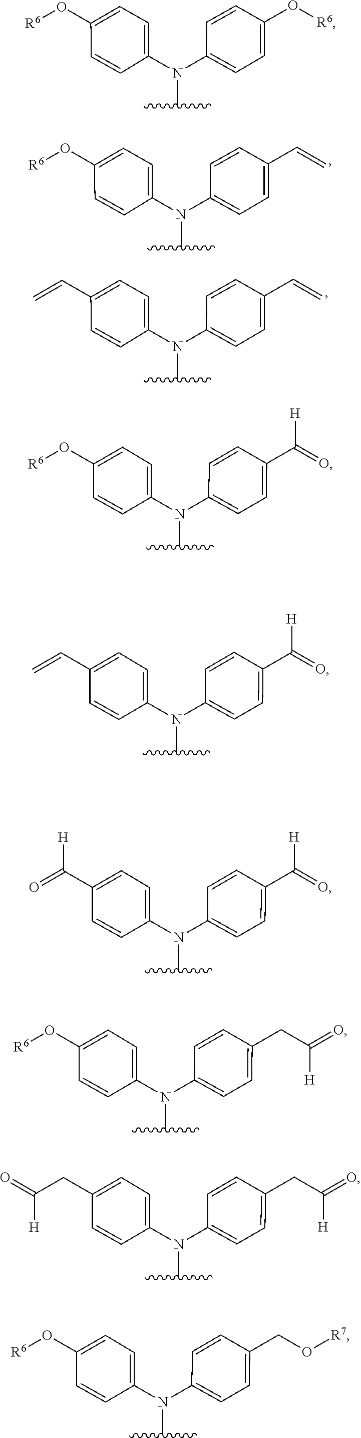

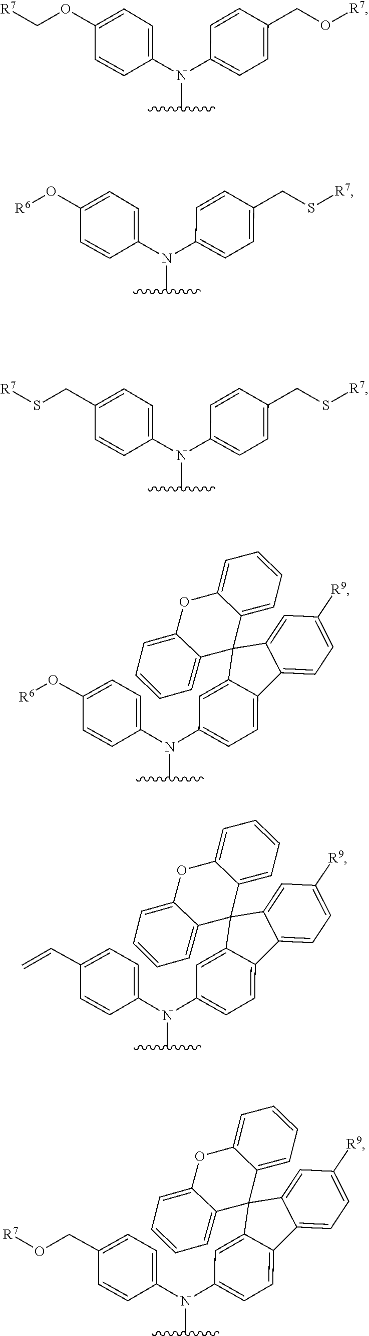

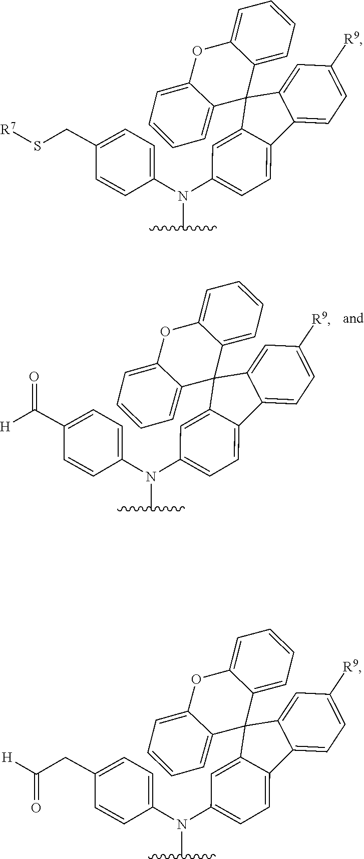

with R⁹ groups being —OR⁶, —R⁷, —OR⁷, or —H and R⁶ and R⁷ groups as described above. These groups may further be substitutents of another structure, such as R⁸ groups of

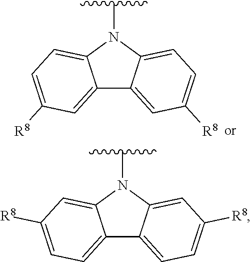

where the wavy bond extending from the nitrogen atom represents attachment between the hole transport structure and the electrolyte 210.

Taken together, the substituents described above for hole transport structures 205, 220, 225, and 230 may optionally correspond to an N,N-di-p-methoxy phenyl amine-based structure. For example, in specific embodiments, hole transport structures 205, 220, 225, and 230 may comprise

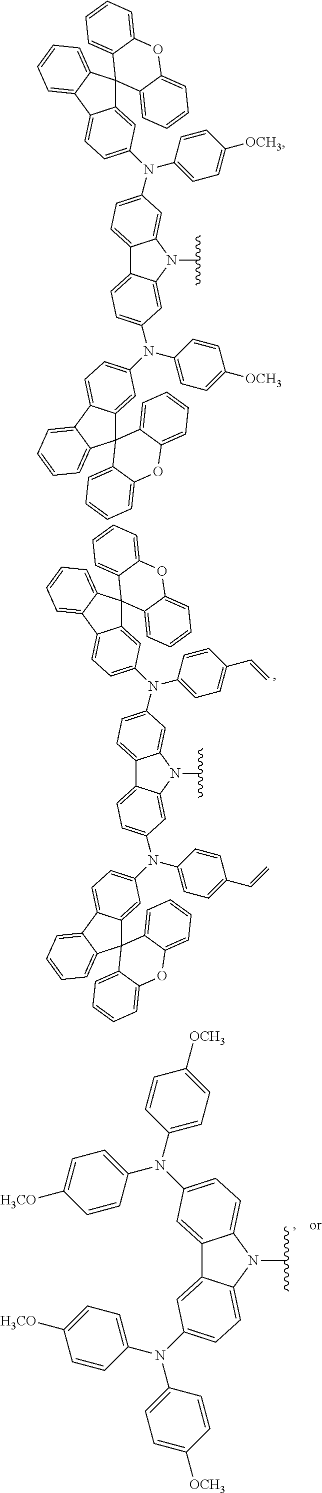

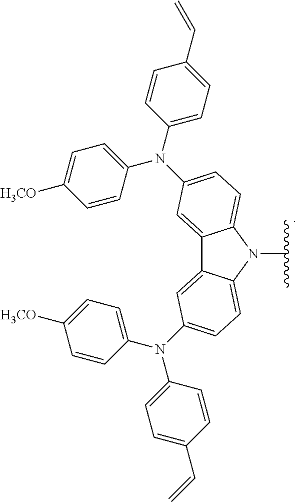 [SMILES: *n1c2ccc(N(c3ccc(C=C)cc3)c3ccc(OC)cc3)cc2c2cc(N(c3ccc(C=C)cc3)c3ccc(OC)cc3)ccc21]

Specific embodiments of hole transport compound 200 may comprise (without inclusion of an anion component of electrolyte 210)

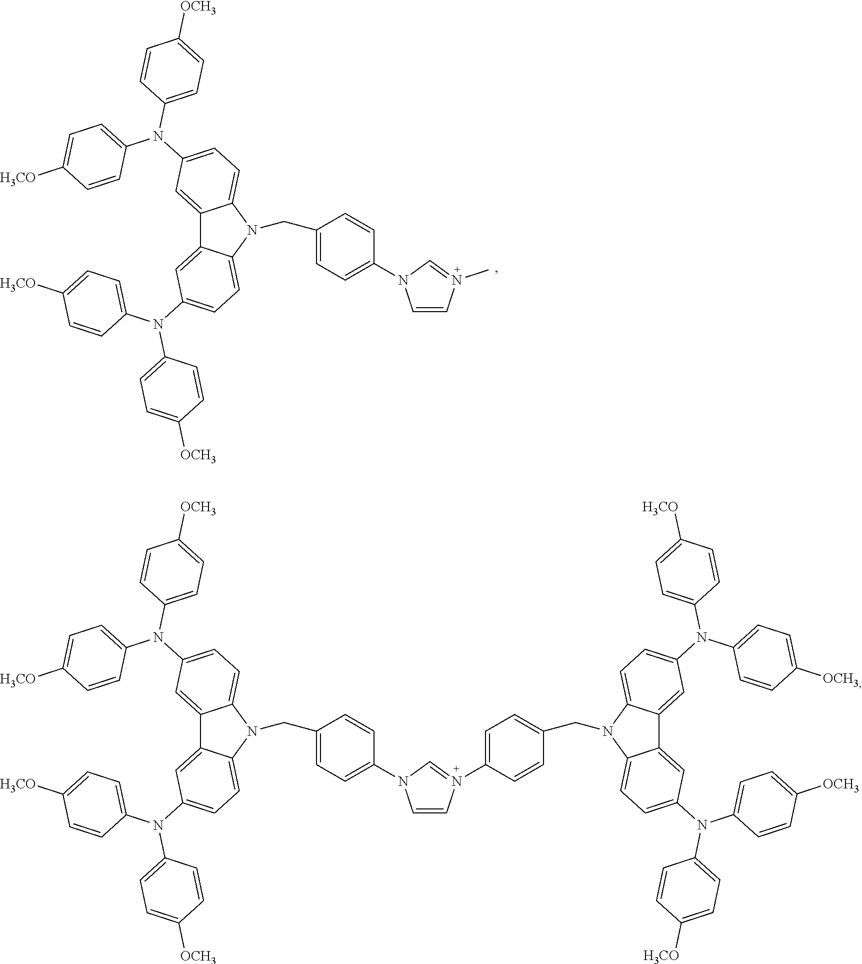

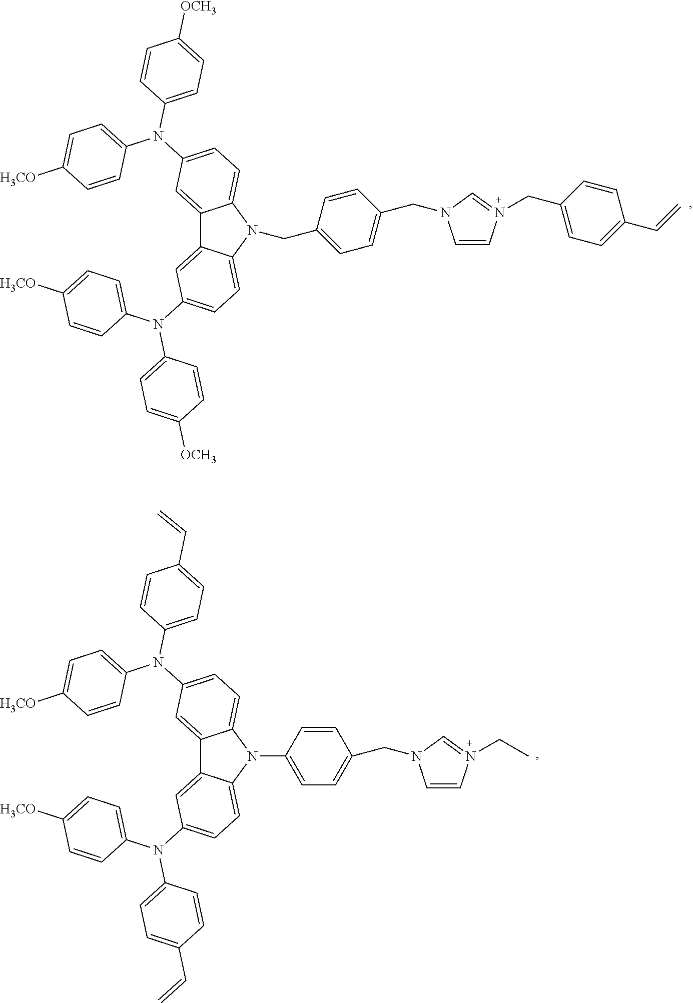

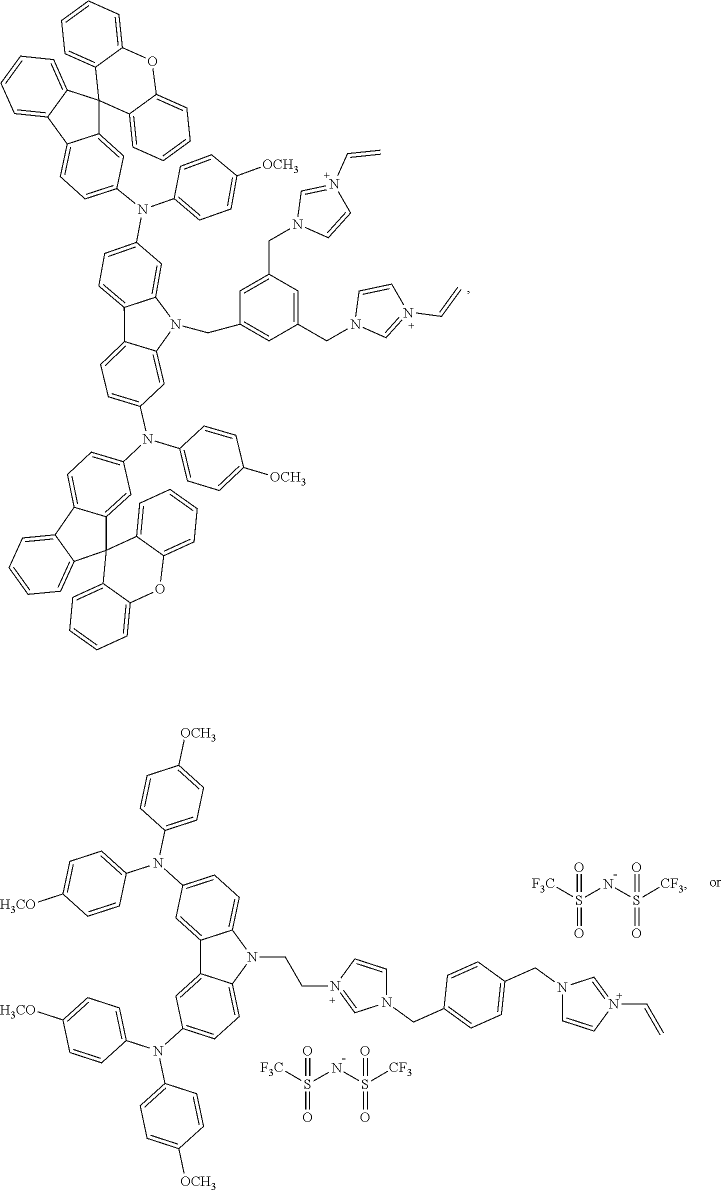

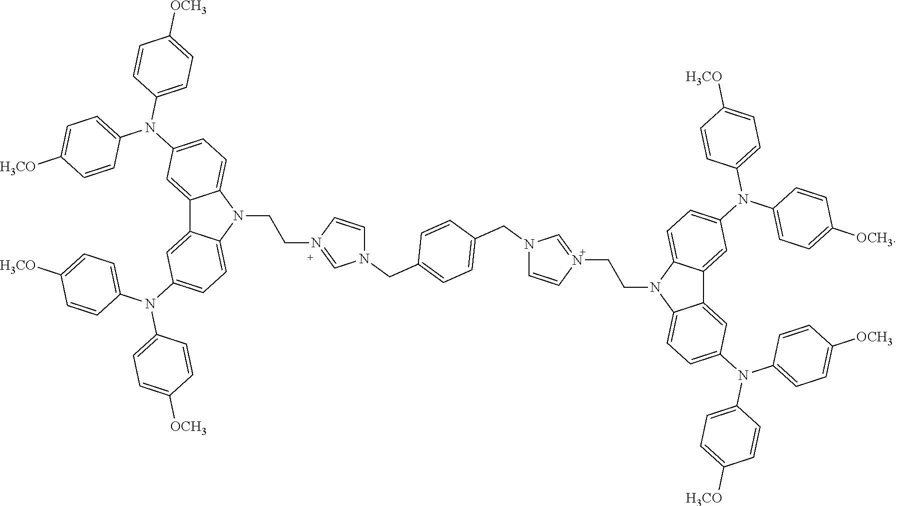

Including the anion component of electrolyte 220, specific embodiments of hole transport compounds 200 may have a formula of having the formula

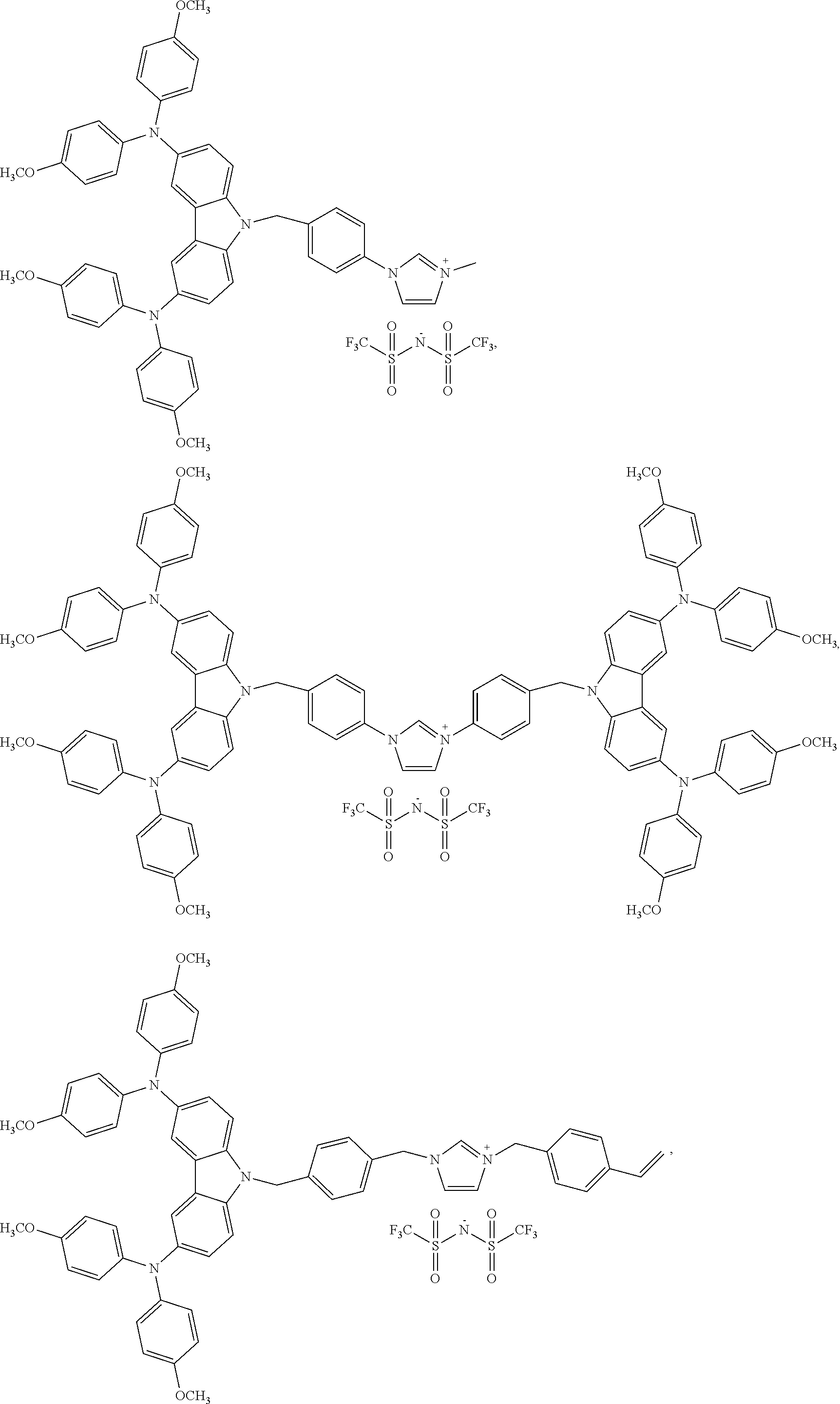

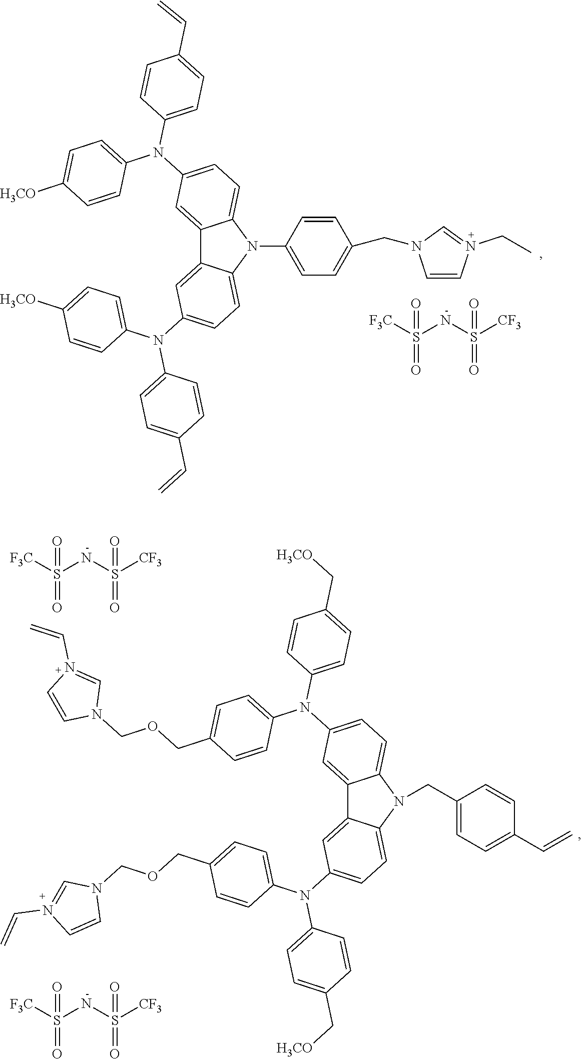

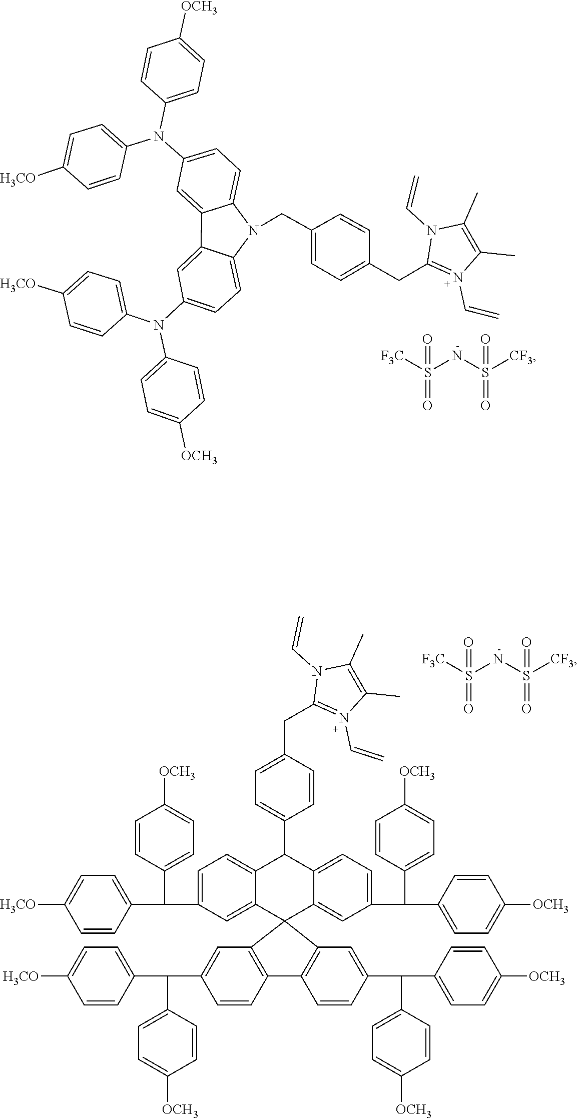

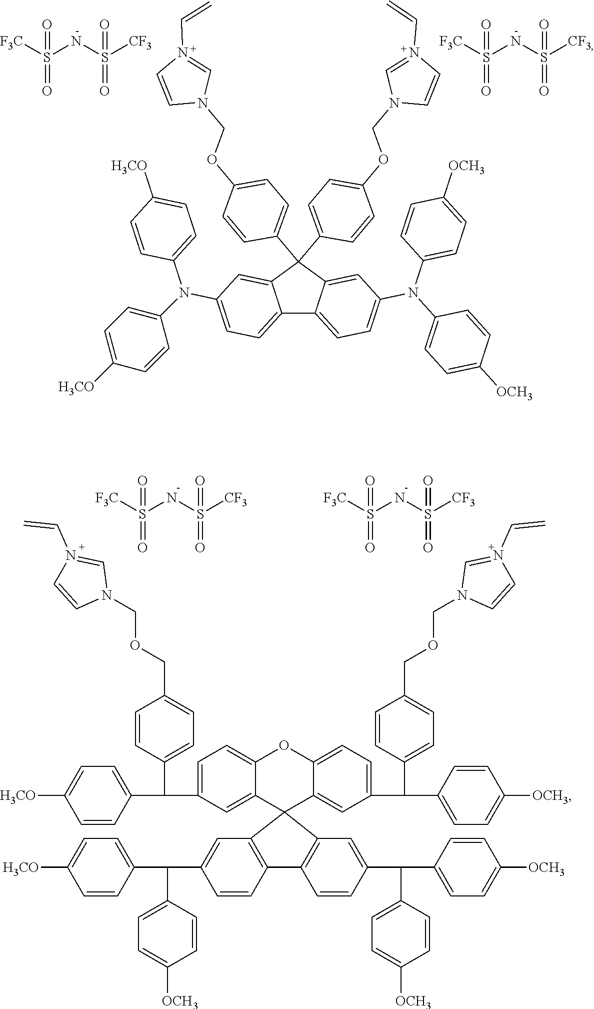

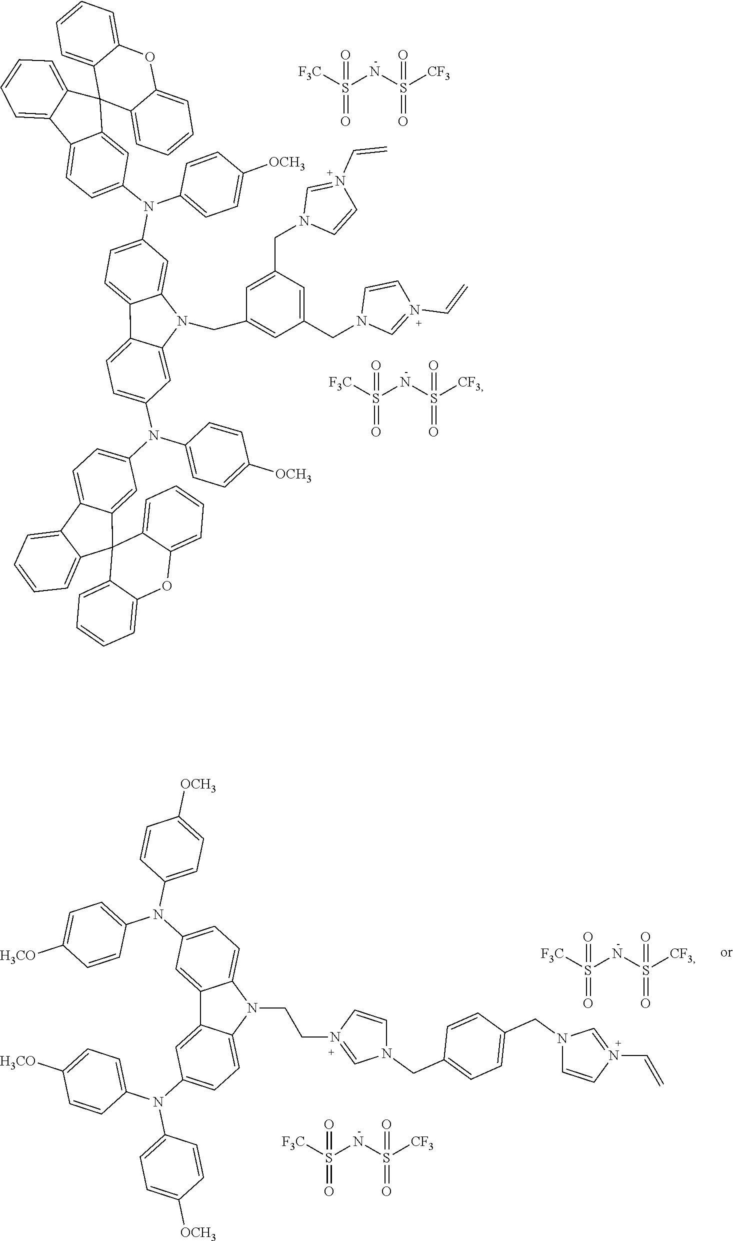

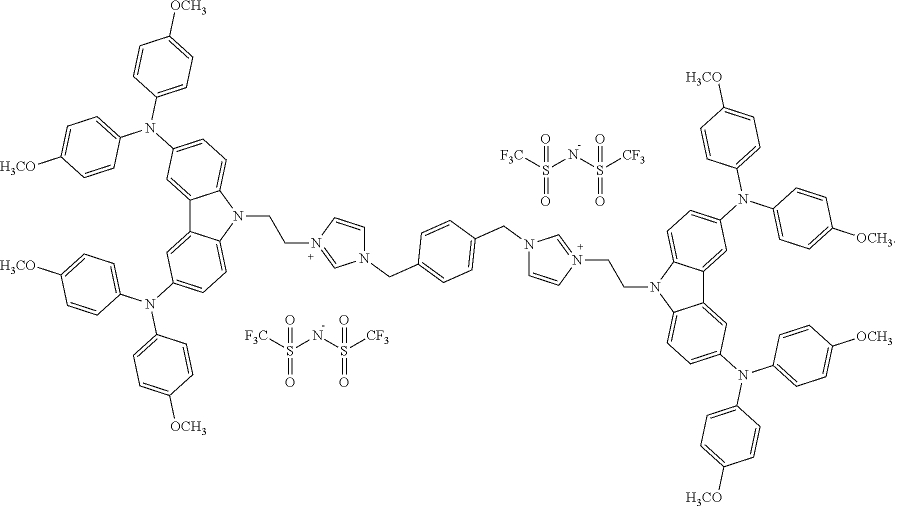

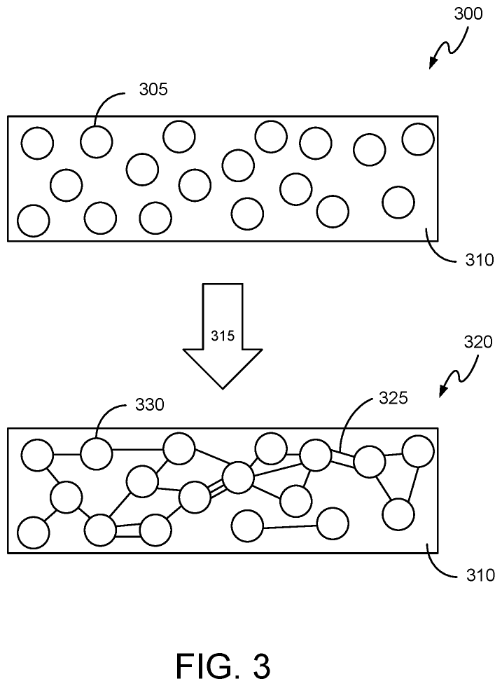

It will be appreciated that hole transport compound 200 may be dissolved in a solvent, such as a polar solvent, allowing dissolution of an ionic bond between a cation portion of hole transport compound 200 and the anion portion of electrolyte 210. FIG. 3 provides a schematic illustration of a hole transport layer 300 including hole transport compound 305, which may be formed by dissolving hole transport compound 305 in or distributing hole transport compound 305 throughout a solvent 310. The hole transport layer 300 may correspond to a thin film, for example, and some or all of solvent 310 may be removed (e.g., by evaporation) when incorporated into a device. Advantageously, hole transport compound 305 may not phase separate from solvent 310.

Depending on the specific structure and configuration of hole transport compound 305, portions of hole transport compound 305 may exhibit different electrostatic characters. Looking back to FIGS. 2A-2D, HTS 205 and 220 may exhibit an overall negative charge character, while the cation portion of electrolyte 210 that is bonded to HTS 205 or 220 may exhibit an overall positive charge character. Such an electrostatic distribution may provide the hole transport compound 200 or 300 with a static dipole moment, enabling hole transport compound 200 or 300 to arranged in a packed or stacked morphology with one another.

Optionally, forming a hole transport layer may include initiating a cross-linking reaction 315 between molecules of hole transport compound 305, such as by exposing hole transport layer 300 to ultraviolet light, visible light, and/or infrared light, and/or heating hole transport layer 300. Cross-linking may transform hole transport layer 300 to cross-linked hole transport layer 320, where cross-links 325, corresponding to covalent bonds, are depicted as present between cross-linked hole transport compound 330. It will be appreciated that the depiction of FIG. 3 is schematic and for illustration purposes only and that multiple cross-links may be present in any form and combination.

Synthetic schemes for preparation of hole transport compounds are also described. In some embodiments, hole transport compounds may be prepared by reacting

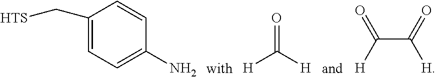

### Optionally

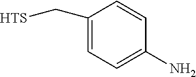

may be prepared by reducing

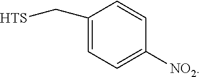

In specific embodiments, hole transport compounds may be prepared by reacting

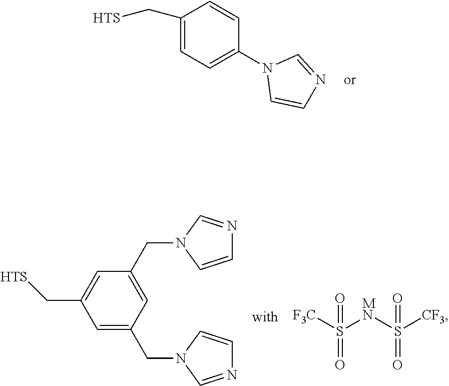

and R¹⁰—Br, wherein M is a metal and R¹⁰ is a C₁-C₂₀ branched or unbranched alkyl group. Optionally,

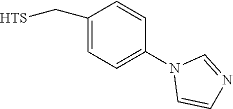

may be prepared by reacting HTS-H or H

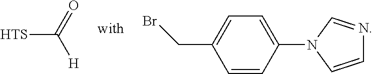

### Optionally,

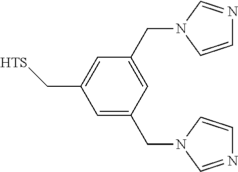

may be prepared by reacting HTS-H or H

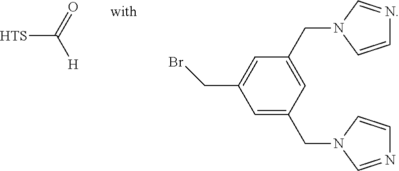

### Optionally,

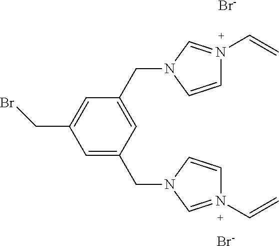

may be prepared by reacting

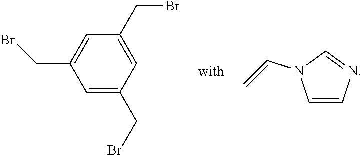

### Optionally

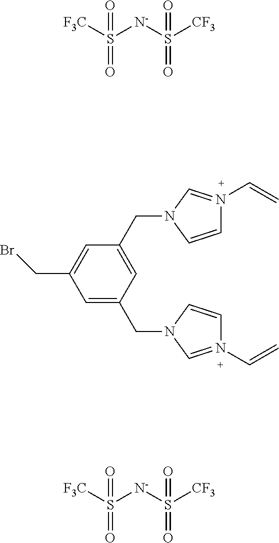

may be prepared by reacting

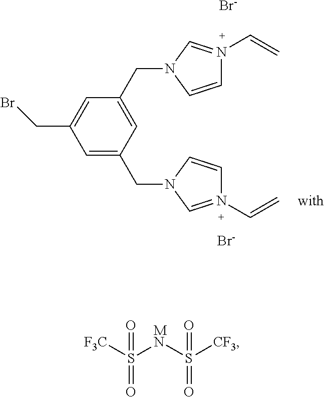

such as in water, with M being a metal, such as lithium. Optionally,

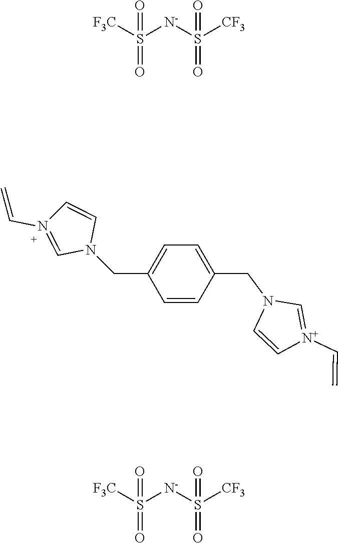

may be prepared by reacting

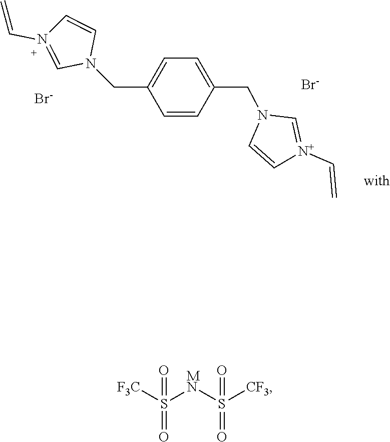

such as in water, with M being a metal, such as lithium. Optionally,

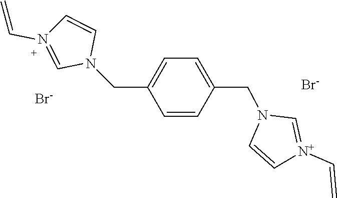

may be prepared by reacting

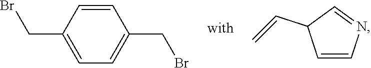

such as in the presence of toluene and/or DMSO.

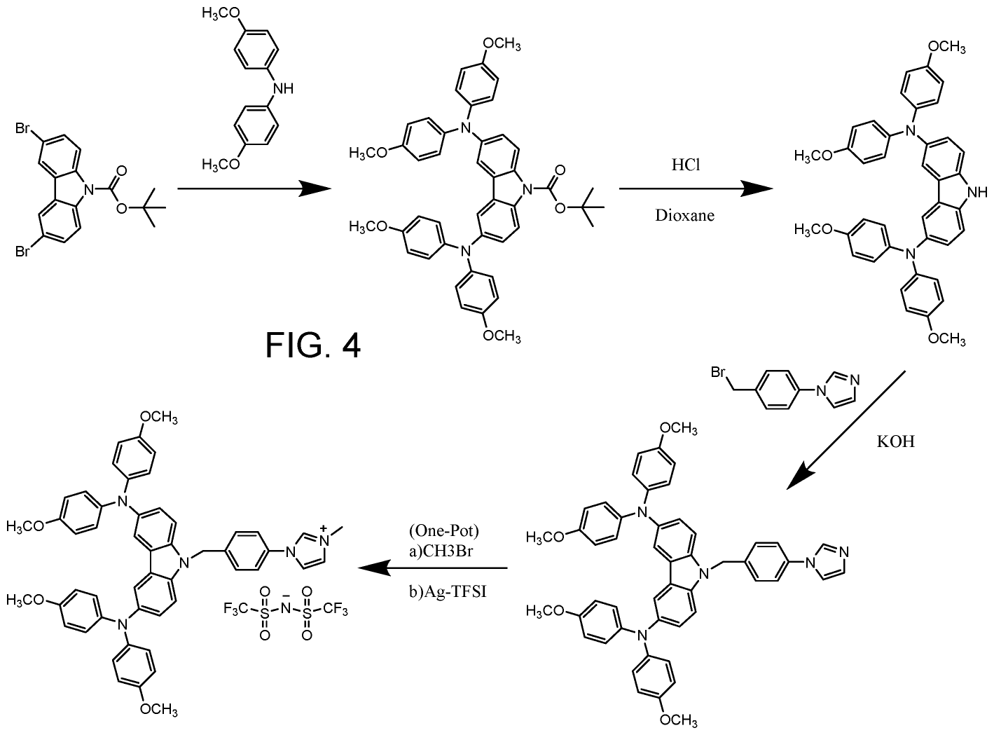

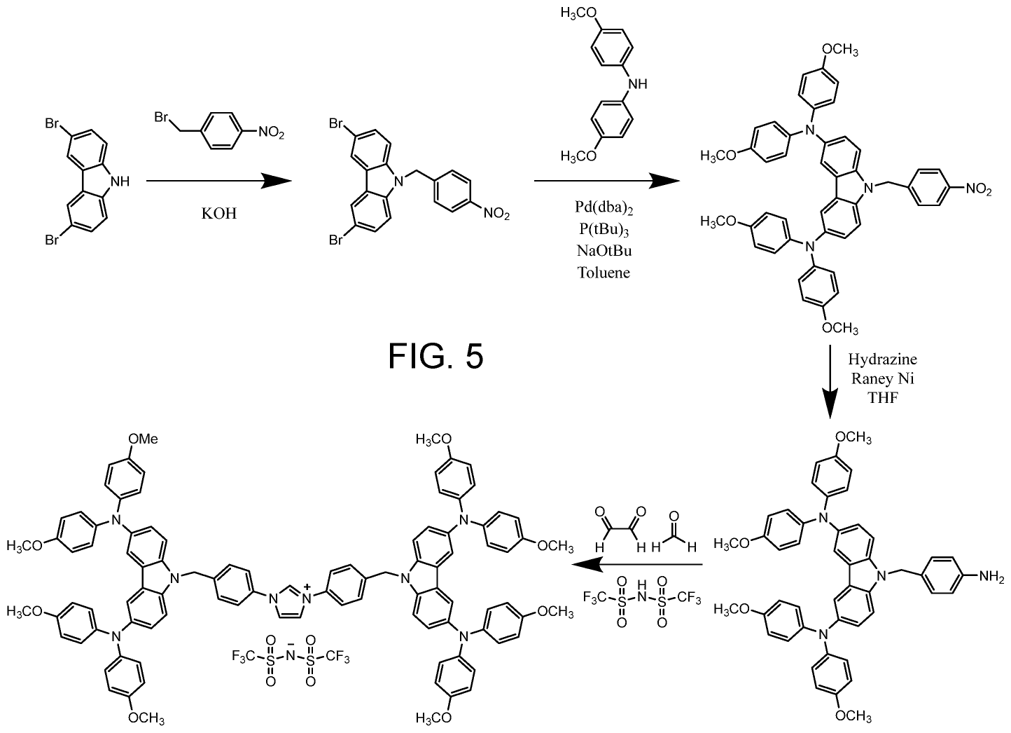

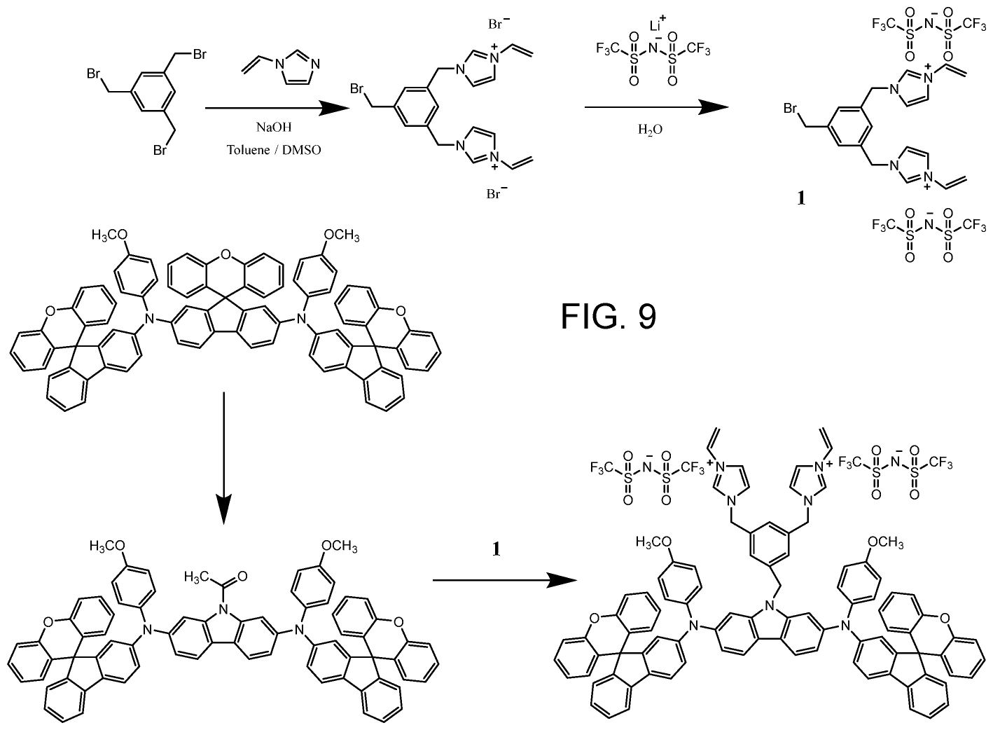

Example synthetic pathways are illustrated in FIGS. 4, 5 and 9. FIGS. 4 and 9 depict synthetic pathways for formation of hole transport compounds with a single hole transport structure bonded to an electrolyte, corresponding to FIG. 2A. FIG. 5 depicts a synthetic pathway for formation of a hole transport compound with two hole transport structures bonded to an electrolyte, corresponding to FIG. 2B.

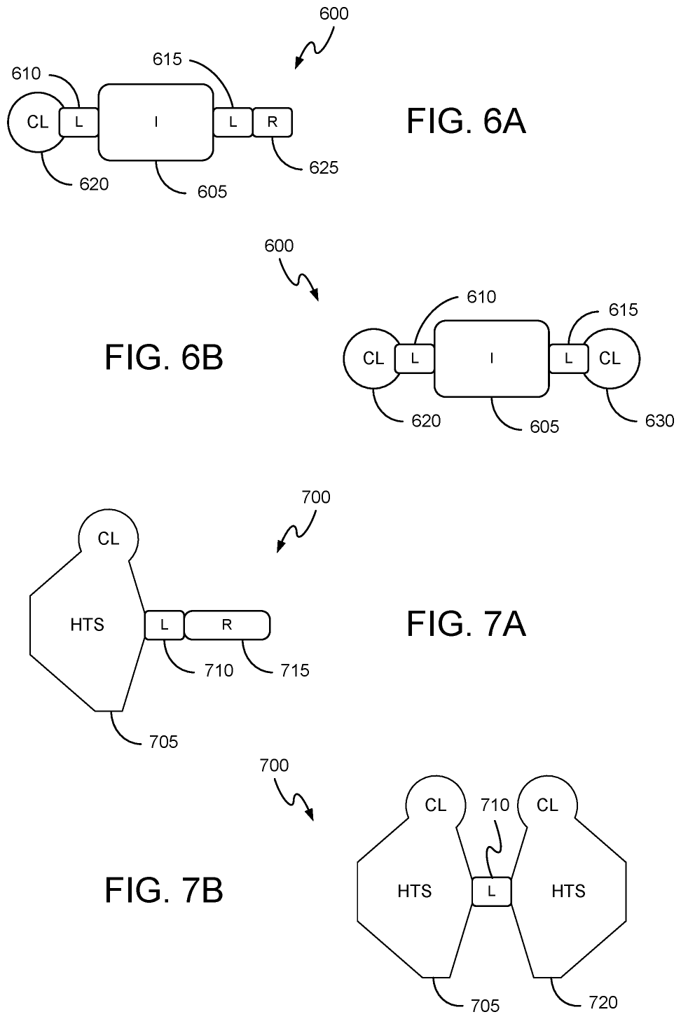

Alternative hole transport materials are described herein, including those comprising a mixture including one or more cross-linkable hole transport compounds. For example, one or more hole transport compounds 200 may be included in the mixture as depicted in FIGS. 2C and 2D. Cross-linkable hole transport compounds may be mixed with a lithium-free electrolyte or, optionally, with a cross-linkable lithium-free electrolyte. FIGS. 6A and 6B provide schematic representations of cross-linkable electrolytes. In FIG. 6A, electrolyte 600 includes an ionic (I) substituent 605, which may comprise a cation component and a cation component. The cation component may be covalently bonded to spacer or linking (L) substituents 610 and 615. Spacer substituent 610 is further covalently bonded to a reactive cross-linkable (CL) group 620. Spacer substituent 615 is further covalently bonded to side group (R) 625. Side group 625 may optionally correspond to a second reactive cross-linkable (CL) group 630, which is depicted in FIG. 6B.

A variety of anion components are useful for ionic substituent 605. For example, the anion component my comprise

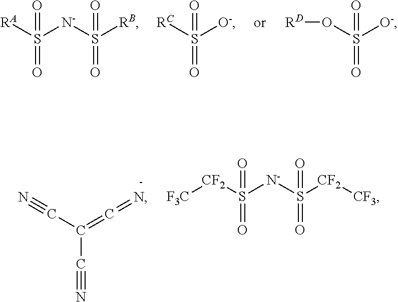

PF₆⁻, BF₄⁻, [SCN]⁻, dimethyl phosphate, [Co(SCN)₄]²⁻, [(C₂F₅)₃PF₃]⁻, or [B(CN)₄]⁻, wherein R^{A}, R^{B}, R^{C}, and R^{D} are independently H, a C₁-C₂₀ alkyl group, a C₁-C₂₀ fluorinated alkyl group, or a C₁-C₂₀ perfluorinated alkyl group. Specific anion components include, but are not limited to,

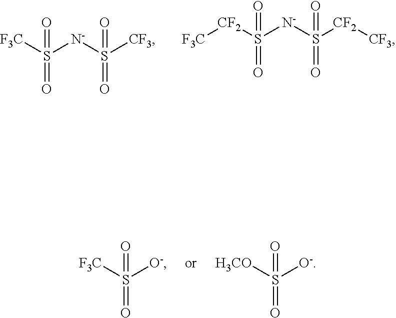

The cation component of ionic substituent 605 is advantageously covalently bonded to spacer substituents 610 and 615. Advantageously, the cation component of ionic substituent 605 is a non-lithium containing cation. A specific cation component of ionic substituent is a bivalent imidazolium group, such as

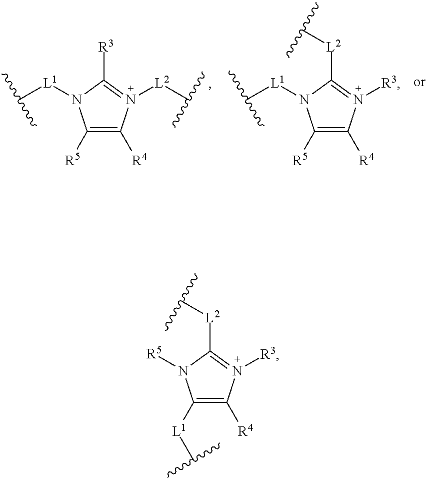

wherein L¹ and L² are independently a spacer or linking group substituent selected from the group including a C₁-C₂₀ branched, unbranched, cyclic, or polycyclic bivalent aliphatic group that is saturated or unsaturated and that is substituted or unsubstituted; and a substituted or unsubstituted bivalent aromatic group that is fused or unfused, and wherein R³, R⁴, and R⁵ are independently a reactive cross-linking group; or H; or a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused. More particularly, L¹ and L² may independently be a branched, unbranched, cyclic, or polycyclic alkylene group that is substituted or unsubstituted; or a branched, unbranched, cyclic, or polycyclic alkenylene group that is substituted or unsubstituted; or a branched, unbranched, cyclic, or polycyclic alkynylene group that is substituted or unsubstituted; or a substituted or unsubstituted arylene group; or a substituted or unsubstituted heteroarylene group. More particularly, L¹ and L² may independently be a branched or unbranched, substituted or unsubstituted fluoroalkylene group; a branched or unbranched, substituted or unsubstituted perfluoroalkyene group; a branched or unbranched, substituted or unsubstituted fluoroalkenylene group; or a substituted or unsubstituted perfluoroaromatic or perfluoroheteroaromatic group. Optionally, L¹ is or comprises

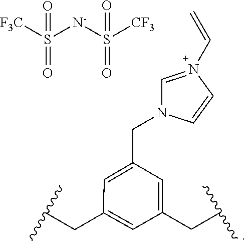

Optionally, L¹ is or comprises

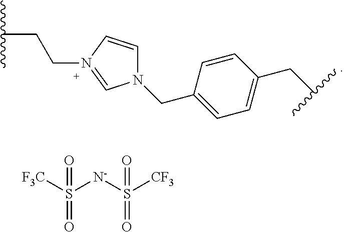

Optionally, R³, R⁴, and R⁵ are independently a branched, unbranched, cyclic, or polycyclic alkyl group that is substituted or unsubstituted; or a branched, unbranched, cyclic, or polycyclic alkenyl group that is substituted or unsubstituted; or a branched, unbranched, cyclic, or polycyclic alkynyl group that is substituted or unsubstituted; or a substituted or unsubstituted aryl group; or a substituted or unsubstituted heteroaryl group. Optionally, R³, R⁴, and R⁵ are independently a branched or unbranched, substituted or unsubstituted fluoroalkyl group; or a branched or unbranched, substituted or unsubstituted perfluoroalkyl group; or a branched or unbranched, substituted or unsubstituted fluoroalkenyl group; or a branched or unbranched, substituted or unsubstituted fluoroalkyne group; or a substituted or unsubstituted perfluoroaromatic or perfluoroheteroaromatic group.

Examples for side group 625 include, but are not limited to, a hydrogen atom, H; a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused. Optionally, side group 625 may be a branched, unbranched, cyclic, or polycyclic alkyl group that is substituted or unsubstituted; a branched, unbranched, cyclic, or polycyclic alkenyl group that is substituted or unsubstituted; a branched, unbranched, cyclic, or polycyclic alkynyl group that is substituted or unsubstituted; a substituted or unsubstituted aryl group; or a substituted or unsubstituted heteroaryl group. Optionally, side group 625 may be a branched or unbranched, substituted or unsubstituted fluoroalkyl group, a branched or unbranched, substituted or unsubstituted perfluoroalkyl group, a branched or unbranched, substituted or unsubstituted fluoroalkenyl group, a branched or unbranched, substituted or unsubstituted fluoroalkyne group, or a substituted or unsubstituted perfluoroaromatic or perfluoroheteroaromatic group. In specific embodiments, side group 625 is a methyl group, an ethyl group, or an aryl group.

A variety of spacer groups 610 and 615 are useful with the electrolytes described herein. For example, spacer groups 610 and 615 may independently be a spacer substituent selected from the group including a C₁-C₂₀ branched, unbranched, cyclic, or polycyclic bivalent aliphatic group that is saturated or unsaturated and substituted or unsubstituted, and a bivalent substituted or unsubstituted aromatic group that is fused or unfused. More particularly, spacer groups 610 and 615 may independently be a branched, unbranched, cyclic, or polycyclic alkylene group that is substituted or unsubstituted; a branched, unbranched, cyclic, or polycyclic alkenylene group that is substituted or unsubstituted; a branched, unbranched, cyclic, or polycyclic alkynylene group that is substituted or unsubstituted; a substituted or unsubstituted arylene group; or a substituted or unsubstituted heteroarylene group. More particularly, spacer groups 610 and 615 may independently be a branched or unbranched, substituted or unsubstituted fluoroalkylene group; a branched or unbranched, substituted or unsubstituted perfluoroalkyene group; a branched or unbranched, substituted or unsubstituted fluoroalkenylene group; or a substituted or unsubstituted perfluoroaromatic or perfluoroheteroaromatic group. In a specific embodiment, spacer groups 610 and 615 correspond to a bivalent aryl group, such as

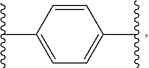 [SMILES: *c1ccc(*)cc1]

or a methylene group (—CH₂—). Optionally, the cation component of ionic group 605 and spacer groups 610 and 615 together comprise

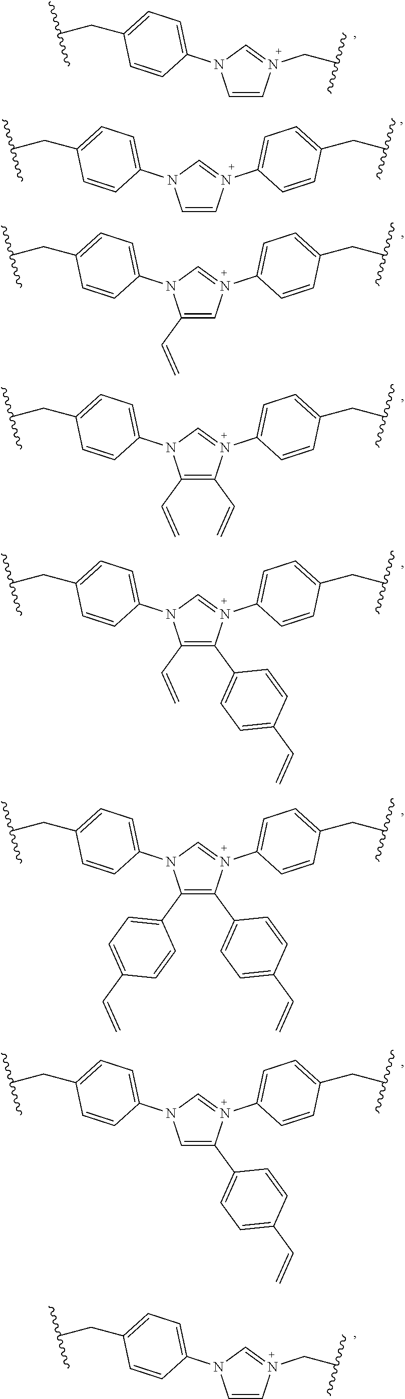

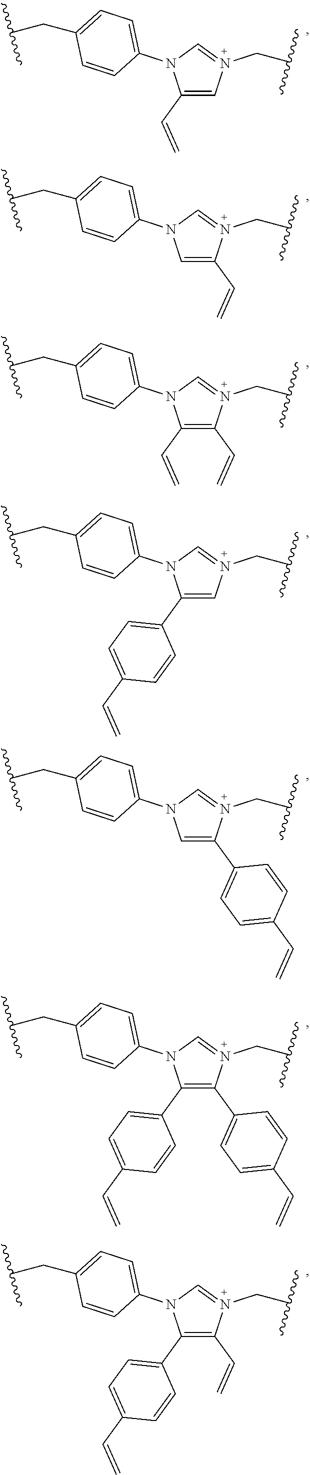

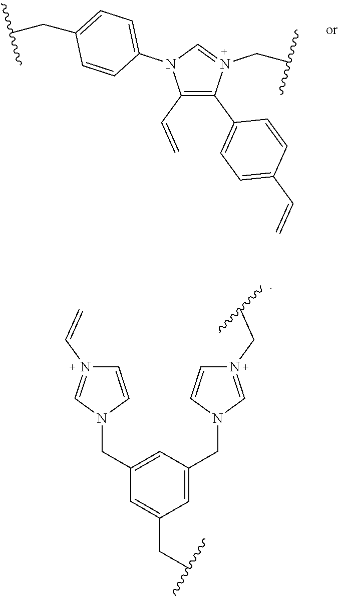

A variety of cross-linkable groups 620 are useful with the electrolyte 600. It will be appreciated that cross-linkable groups on different electrolyte 600 molecules may be reactive with one another or induced to react with one another upon exposure to a sufficient energy source (such as heat or ultraviolet light, visible light, or infrared light). It will further be appreciated that cross-linkable groups on electrolyte 600 molecules may be reactive with cross-linkable groups on a cross-linkable hole transport compound or induced to react with one another upon exposure to a sufficient energy source (such as heat or ultraviolet light, visible light, or infrared light). Specific cross-linkable groups 620 include, but are not limited to, —NH₂, —OH, —SH, —SiCl₃, —Si(OH)₃,

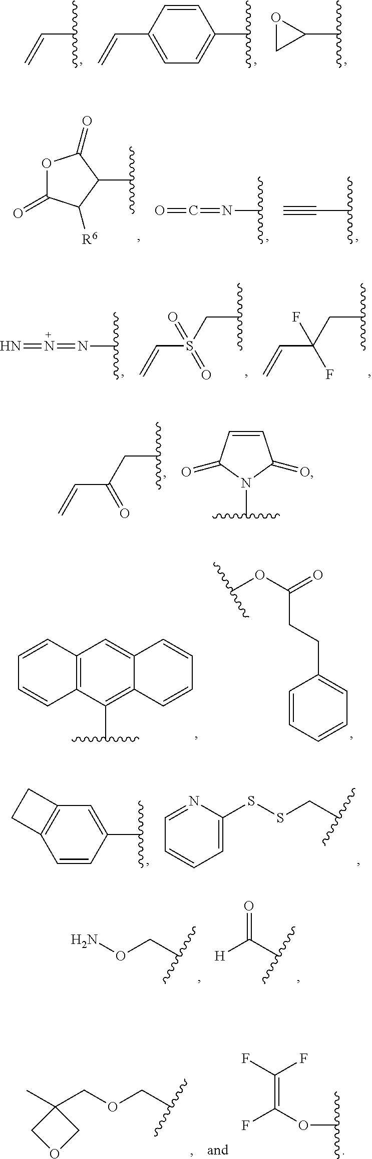

FIG. 7A depicts a schematic representation of a hole transport compound 700 comprising a hole transport structure 705 that is cross-linkable (CL) or includes a cross-linkable group. Hole transport compound 700 also includes a spacer group 710 and a side group (R) 705. Useful side groups include, but are not limited to H, a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused. Side group 715 may optionally correspond to a second hole transport structure 720 that is cross-linkable (CL), which is depicted in FIG. 7B.

A variety of spacer groups 710 are useful with the hole transport structure 700. For example, spacer group 710 may be a spacer substituent selected from the group including a C₁-C₂₀ branched, unbranched, cyclic, or polycyclic bivalent aliphatic group that is saturated or unsaturated and substituted or unsubstituted, and a bivalent substituted or unsubstituted aromatic group that is fused or unfused. More particularly, spacer group 710 may be a branched, unbranched, cyclic, or polycyclic alkylene group that is substituted or unsubstituted; a branched, unbranched, cyclic, or polycyclic alkenylene group that is substituted or unsubstituted; a branched, unbranched, cyclic, or polycyclic alkynylene group that is substituted or unsubstituted; a substituted or unsubstituted arylene group; or a substituted or unsubstituted heteroarylene group. More particularly, spacer group 710 may be a branched or unbranched, substituted or unsubstituted fluoroalkylene group; a branched or unbranched, substituted or unsubstituted perfluoroalkyene group; a branched or unbranched, substituted or unsubstituted fluoroalkenylene group; or a substituted or unsubstituted perfluoroaromatic or perfluoroheteroaromatic group. In specific embodiment, spacer group correspond to a methylene group (—CH₂—) or a bivalent aryl group, such as

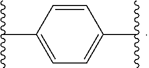 [SMILES: *c1ccc(*)cc1]

Example hole transport structures 705 or 720 include, but are not limited to, organic or heterorganic groups exhibiting a band gap of between 1.4 eV and 3.5 eV and/or an ionization potential of between 4.5 eV and 5.5 eV. Such electronic properties are useful for allowing the hole transport structures 705 or 720 to provide or receive electrons/holes from a photoactive material, such as a perovskite photoactive layer. Such a band gap may correspond to an energy difference between a highest occupied molecular orbital of the hole transport structure and a lowest unoccupied molecular orbital of the hole transport structure, which may allow for favorable electron/hole transfer between the hole transport structure and the photoactive material.

As depicted in FIGS. 7A and 7B, HTS 705 and 720 are cross-linkable or include one or more reactive cross-linkable substituents. It will be appreciated that cross-linkable groups on different cross-linkable hole transport structures may be reactive with one another or induced to react with one another or with cross-linkable groups on a cross-linkable electrolyte, such as electrolyte 600, upon exposure to a sufficient energy source (such as heat or ultraviolet light). For example, HTS 705 or 720 may include one or more substituents selected from the group including

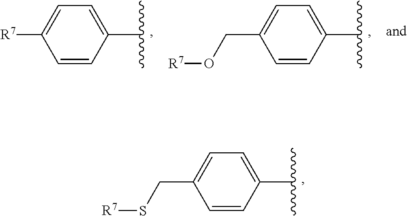

wherein each R⁷ is independently a reactive cross-linkable substituent. In specific examples, R⁷ is selected from the group including: —NH₂, —OH, —SH, —SiCl₃, —Si(OH)₃,

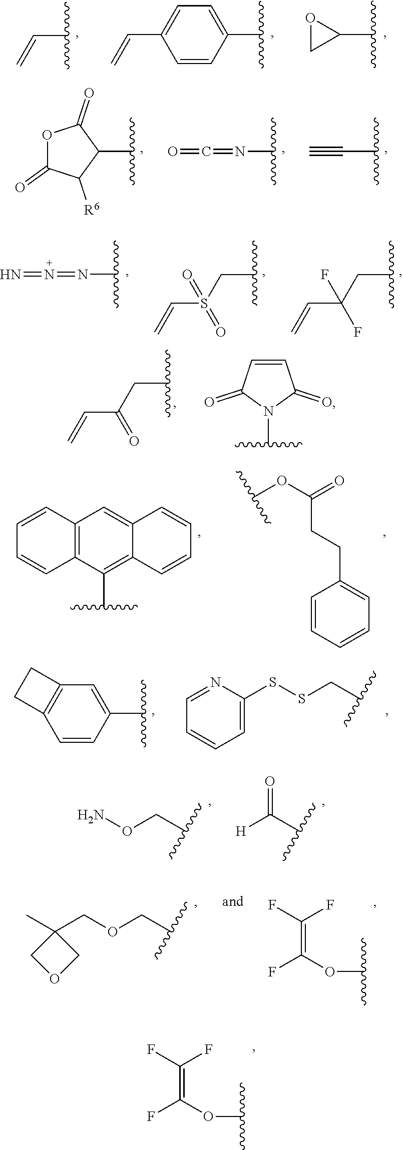

wherein R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group. Optionally, R⁶ is a branched, unbranched, cyclic, or polycyclic alkyl group that is substituted or unsubstituted; a branched, unbranched, cyclic, or polycyclic alkenyl group that is substituted or unsubstituted; a branched, unbranched, cyclic, or polycyclic alkynyl group that is substituted or unsubstituted; a substituted or unsubstituted aryl group; or a substituted or unsubstituted heteroaryl group.

Useful hole transport structures include those comprising one or more homocyclic, heterocyclic, aromatic, or heteroaromatic substituents that are fused or unfused. Example heterocyclic substituents may comprise one or more of oxygen, sulfur, selenium, tellurium, nitrogen, phosphorus, silicon, germanium, boron, aluminum, a transition metal, or a transition metal oxide. Specific example aromatic or heteroaromatic substituents comprise a phenyl group, a fused phenyl group, a heterocycle, or a fused heterocycle. Optionally, the hole transport structure comprises a non-aromatic heterocycle fused to an aromatic group. Example components for HTS 705 or 720 include a triarylamine, a carbazole, a furan, a thiophene, a pyridine, or combinations of these. Aromatic, heteroaromatic, or amine groups of a hole transport structures may be functionalized by various substituents. For example, HTS 705 or 720 may include one or more substituents selected from the group including

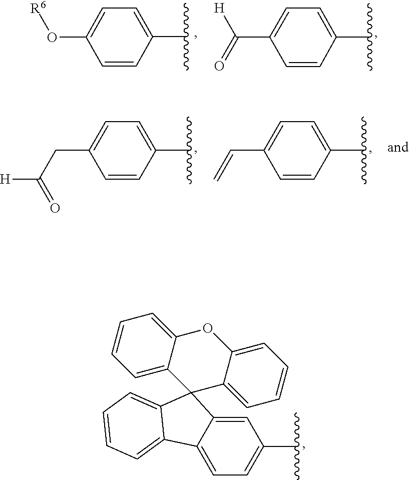

wherein each R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group. Optionally, each R⁶ is a branched, unbranched, cyclic, or polycyclic alkyl group that is substituted or unsubstituted; a branched, unbranched, cyclic, or polycyclic alkenyl group that is substituted or unsubstituted; a branched, unbranched, cyclic, or polycyclic alkynyl group that is substituted or unsubstituted; a substituted or unsubstituted aryl group; or a substituted or unsubstituted heteroaryl group.

Hole transport structures 705 and 720 may include one or more substituents independently selected from the group including:

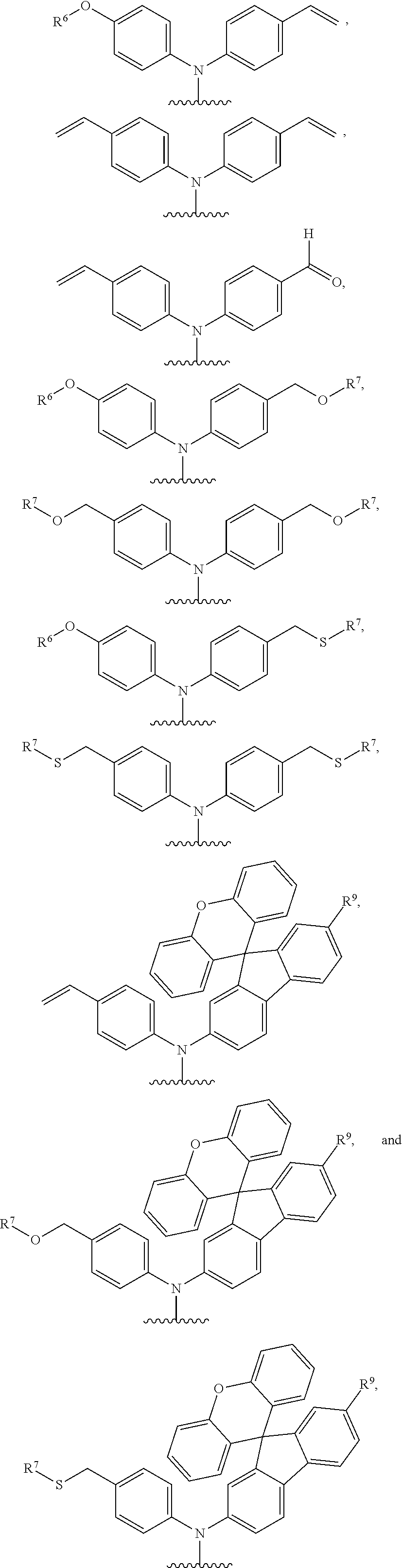

with R⁹ groups being —OR⁶, —R⁷, —OR⁷, or —H and R⁶ and R⁷ groups as described above. These groups may further be substituents of another structure, such as R groups of

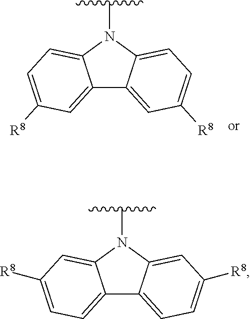

where the wavy bond extending from the nitrogen atom represents attachment between the hole transport structure and the spacer group 710.

Taken together, the substituents described above for hole transport structures 705 and 720 may optionally correspond to an N,N-di-p-methoxy phenyl amine-based structure. For example, in specific embodiments, hole transport structures 705 and 720 may comprise or have a formula of

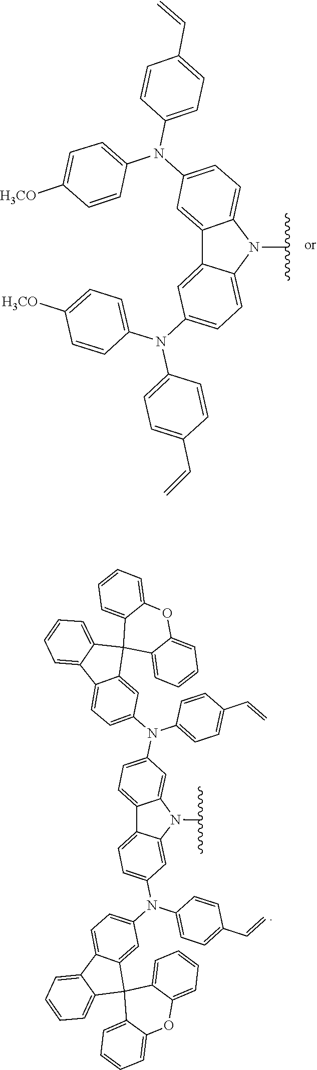 [SMILES: *n1c2ccc(N(c3ccc(C=C)cc3)c3ccc(OC)cc3)cc2c2cc(N(c3ccc(C=C)cc3)c3ccc(OC)cc3)ccc21 ; *n1c2cc(N(c3ccc(C=C)cc3)c3ccc4c(c3)C3(c5ccccc5Oc5ccccc53)c3ccccc3-4)ccc2c2ccc(N(c3ccc(C=C)cc3)c3ccc4c(c3)C3(c5ccccc5Oc5ccccc53)c3ccccc3-4)cc21]

In specific examples, hole transport compound 700 has a formula of

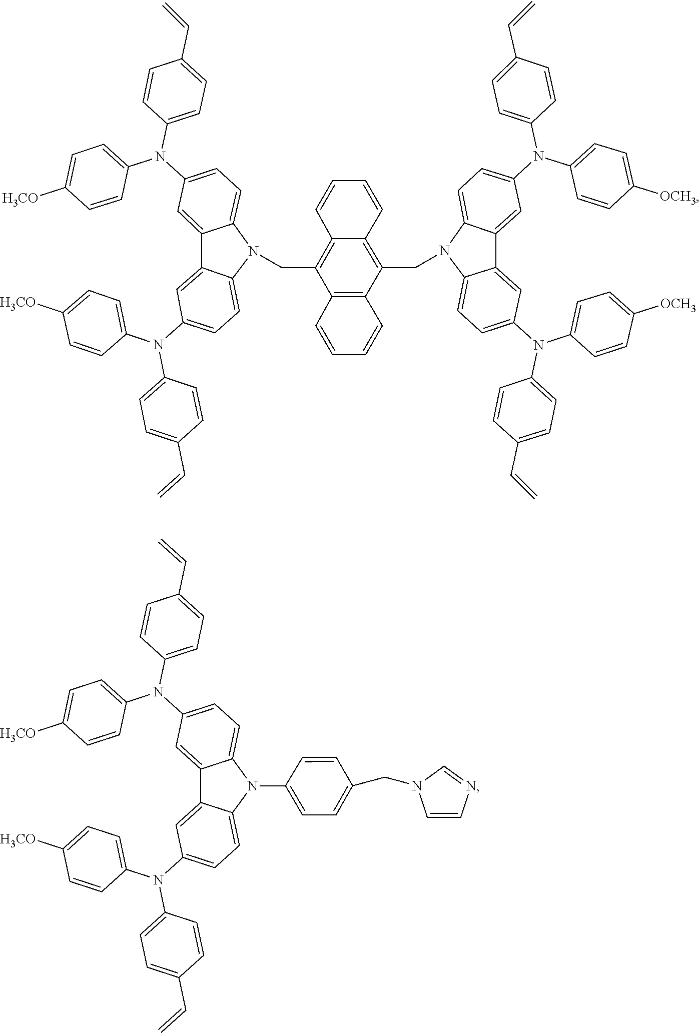

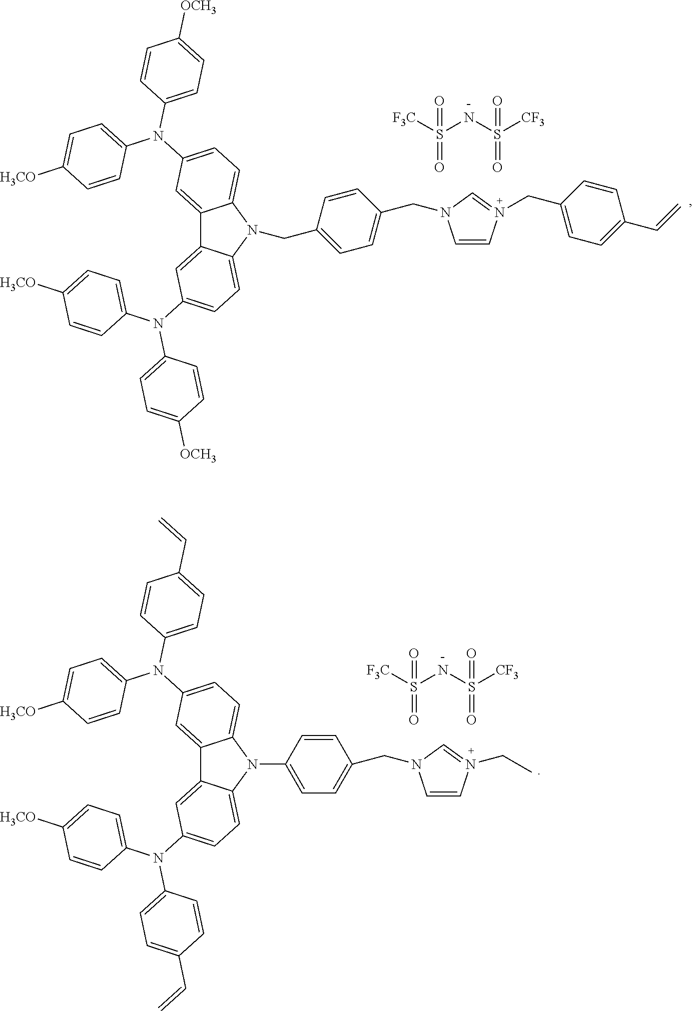

Optionally, spacer group 710 comprises an electrolyte component, such that hole transport compound 700 may correspond to hole transport compound 200 of FIG. 2C or FIG. 2D. Specific embodiments of hole transport compound 700 may have a formula of

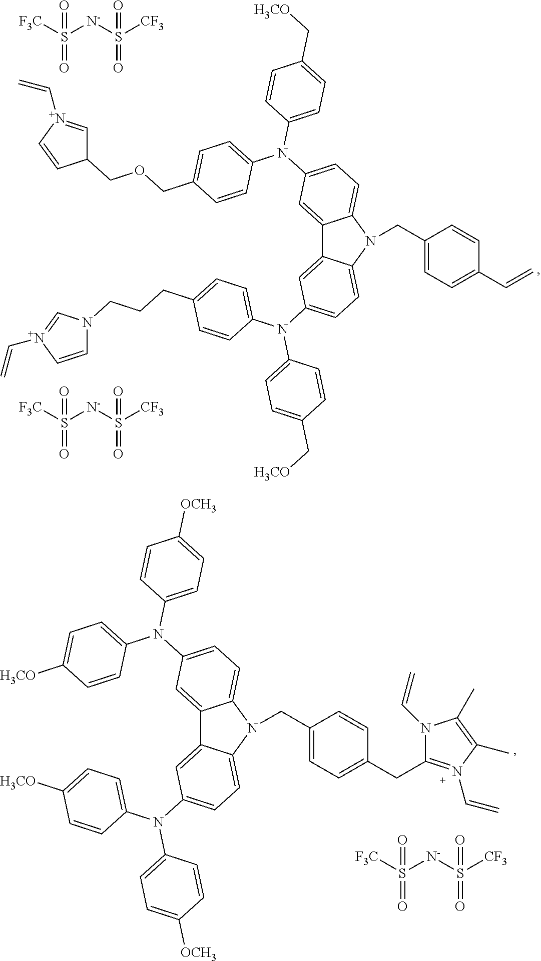

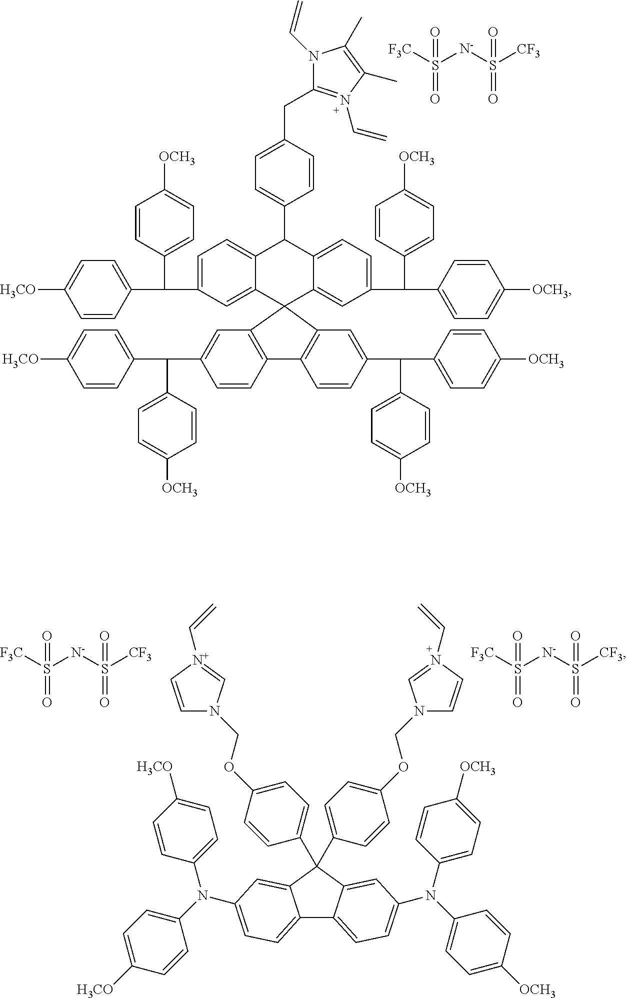

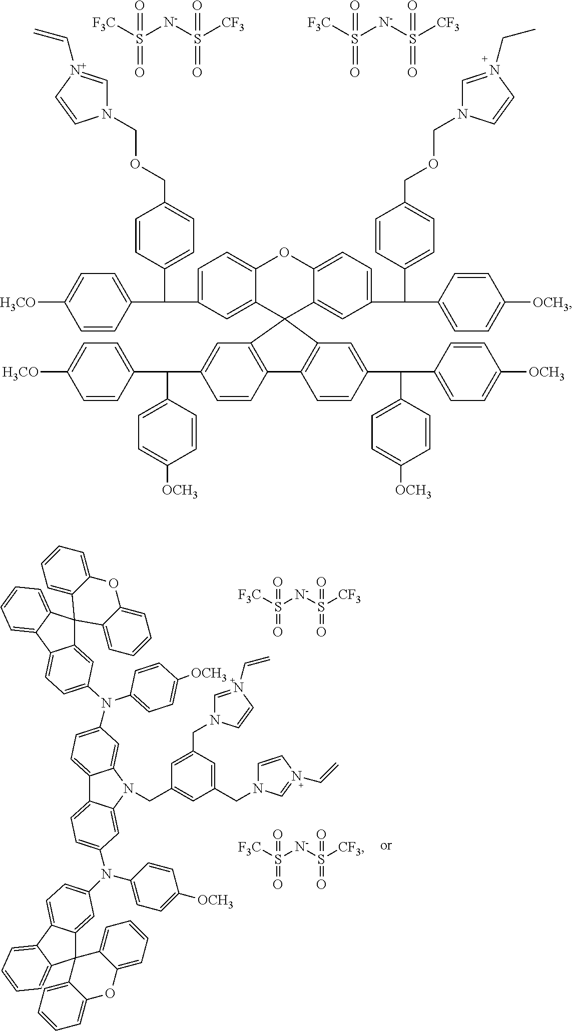

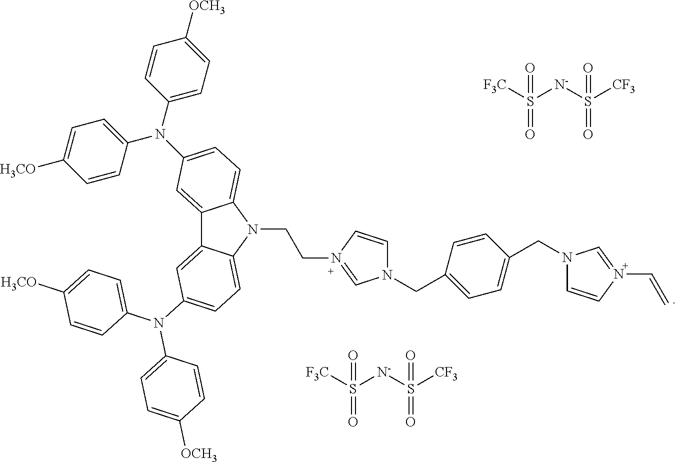

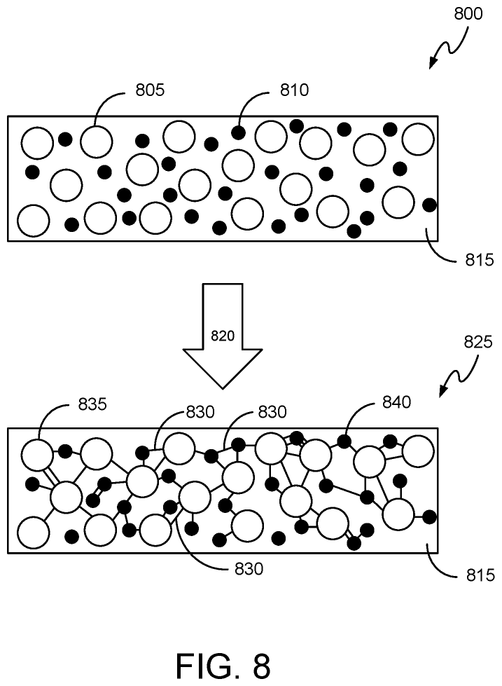

It will be appreciated that a mixture of hole transport compound 700 and electrolyte 600 may be dissolved in a solvent, such as a polar solvent, allowing dissolution of an ionic bond between a cation portion of ionic substituent 605 and an anion portion of ionic substituent 605 of electrolyte 600. FIG. 8 provides a schematic illustration of a hole transport layer 800 including hole transport compound 805, which may be formed by dissolving hole transport compound 805 and electrolyte 810 in or distributing hole transport compound 805 and electrolyte 810 throughout a solvent 815. The hole transport layer 800 may correspond to a thin film, for example, and some or all of solvent 815 may be removed (e.g., by evaporation) when incorporated into a device. Advantageously, hole transport compound 805 and electrolyte 810 may not phase separate from one another and/or the solvent 815.

Optionally, forming a hole transport layer may include initiating a cross-linking reaction 820 between hole transport compound 805 and electrolyte 810, between molecules of hole transport compound 805, and/or between molecules of electrolyte 810, such as by exposing hole transport layer 800 to ultraviolet light or heating hole transport layer 800. Cross-linking may transform hole transport layer 300 to cross-linked hole transport layer 825, where cross-links 830, corresponding to covalent bonds, are depicted as present between cross-linked hole transport compound 835, between cross-linked electrolyte 840, or between the two. It will be appreciated that the depiction of FIG. 8 is schematic and for illustration purposes only and that multiple cross-links may be present in any form and combination.

### STATEMENTS REGARDING INCORPORATION BY REFERENCE AND VARIATIONS

All references throughout this disclosure, for example patent documents including issued or granted patents or equivalents; patent application publications; and non-patent literature documents or other source material; are hereby incorporated by reference herein in their entireties, as though individually incorporated by reference.

All patents and publications mentioned in this disclosure are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”.

Every formulation or combination of components described or exemplified can be used to practice the invention, unless otherwise stated. Specific names of materials are intended to be exemplary, as it is known that one of skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A compound having a formula:
HTS-E-R¹, wherein:
E is a lithium-free electrolyte having an anion component and a cation component, the cation component covalently bonded to HTS and R¹;
HTS is a hole transport structure;
R¹ is HTS; or H; or R²; or a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused; and
R² is a reactive cross-linking group.

2. The compound of claim 1, wherein R¹ is a branched, unbranched, cyclic, or polycyclic alkyl group that is substituted or unsubstituted; a branched, unbranched, cyclic, or polycyclic alkenyl group that is substituted or unsubstituted; a branched, unbranched, cyclic, or polycyclic alkynyl group that is substituted or unsubstituted; a substituted or unsubstituted aryl group; or a substituted or unsubstituted heteroaryl group.

3. The compound of claim 1, wherein the cation component comprises 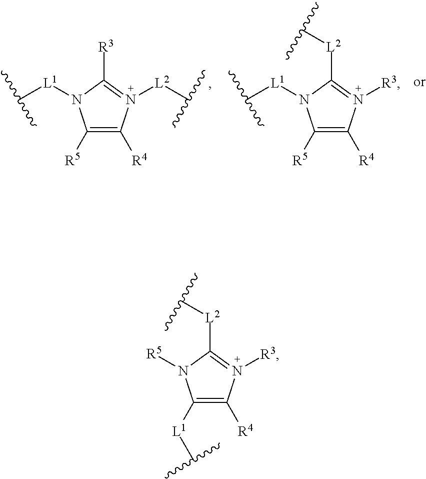
wherein L¹ and L² are independently a spacer substituent selected from a C₁-C₂₀ branched, unbranched, cyclic, or polycyclic bivalent aliphatic group that is saturated or unsaturated and that is substituted or unsubstituted; or a substituted or unsubstituted bivalent aromatic group that is fused or unfused, and wherein R³, R⁴, and R⁵ are independently a reactive cross-linking group; or H; or a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused.

4. The compound of claim 1, wherein each R² is independently a reactive cross-linking group selected from:
—NH₂, —OH, —SH, —SiCl₃, —Si(OH)₃, 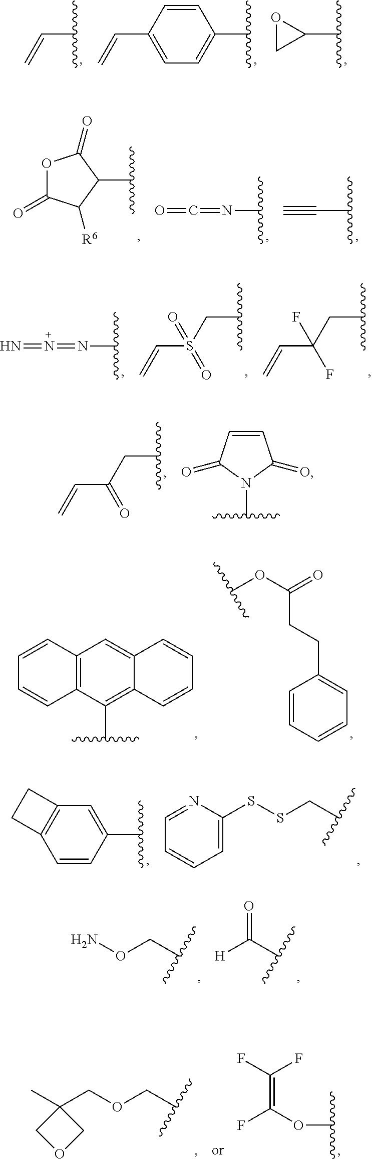
wherein R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group.

5. The compound of claim 1, wherein E-R¹ comprises

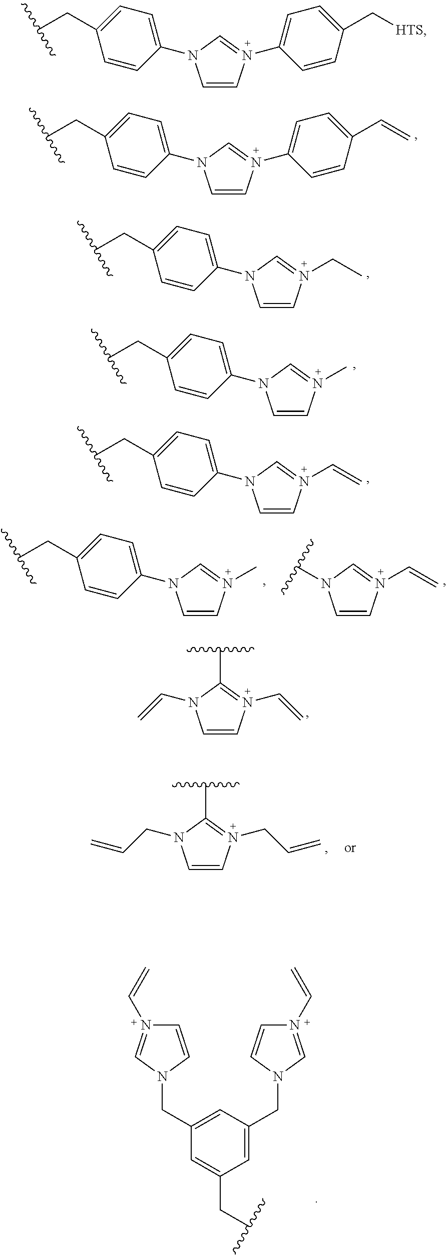

6. The compound of claim 1, wherein the anion component comprises 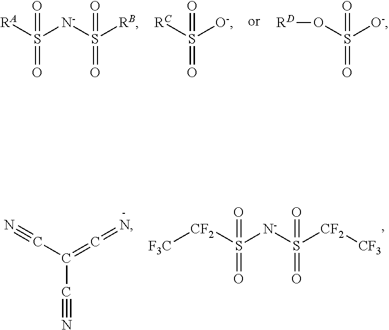
BF₄⁻, [SCN]⁻, dimethyl phosphate, [Co(SCN)₄]²⁻, [(C₂F₅)₃PF₃]⁻, or [B(CN)₄]⁻, wherein R^{A}, R^{B}, R^{C}, and R^{D} are independently H, a C₁-C₂₀ alkyl group, a C₁-C₂₀ fluorinated alkyl group, or a C₁-C₂₀ perfluorinated alkyl group.

7. The compound of claim 1, wherein the anion component is ionically bound to the cation component.

8. The compound of claim 1, wherein HTS comprises 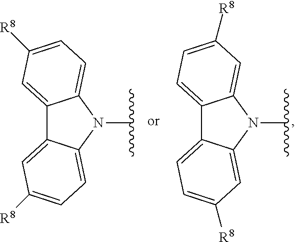
wherein each R⁸ is independently: 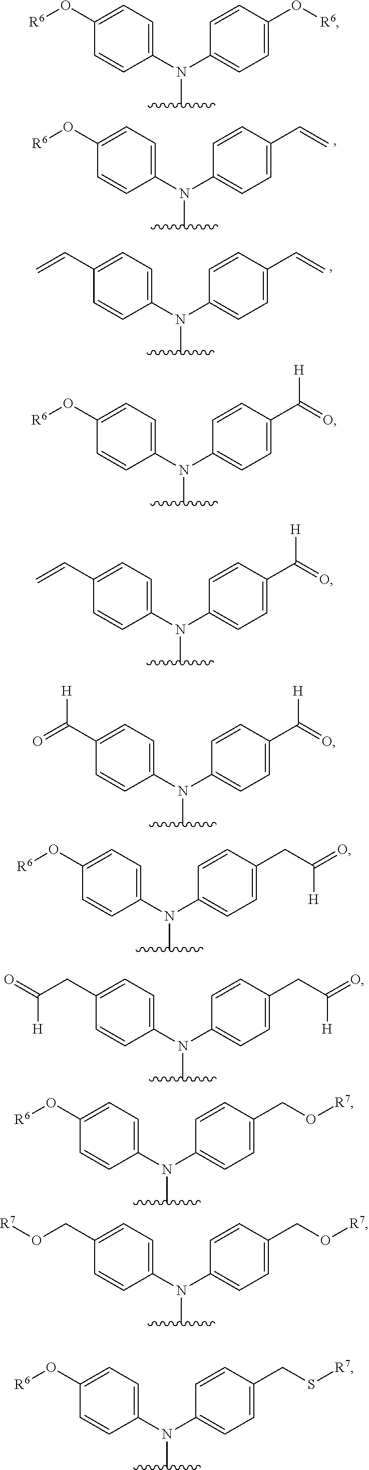 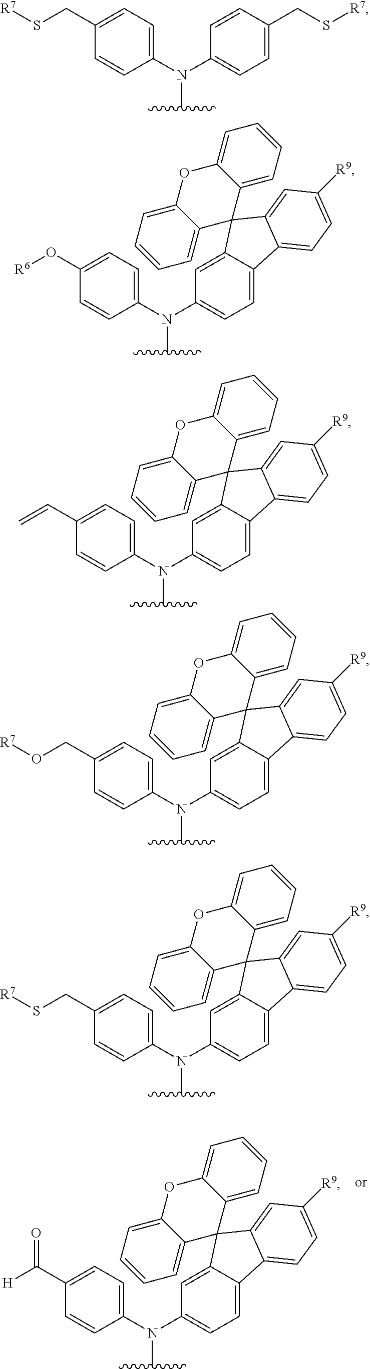 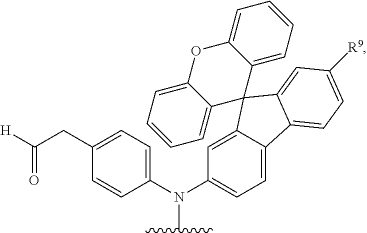
wherein each R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group,
wherein each R⁷ is independently a reactive cross-linkable substituent, and
wherein R⁹ is —OR⁶, —R⁷, —OR⁷, or —H.

9. The compound of claim 1, wherein HTS-E-R¹ comprises

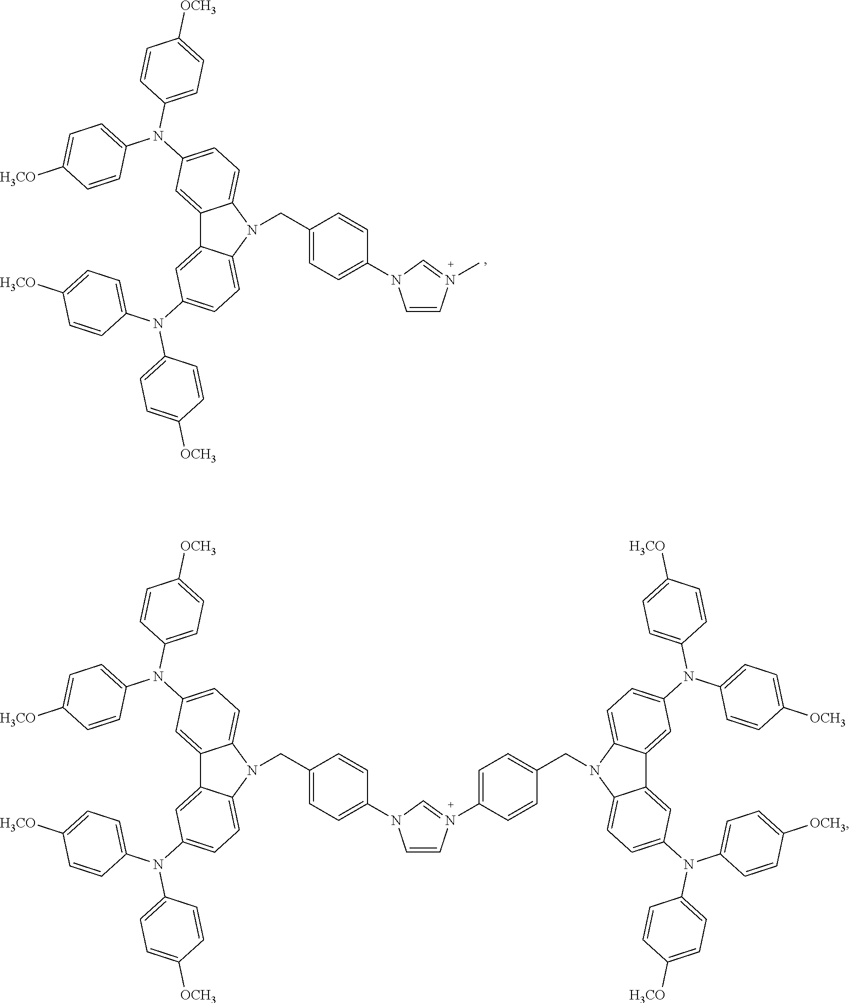

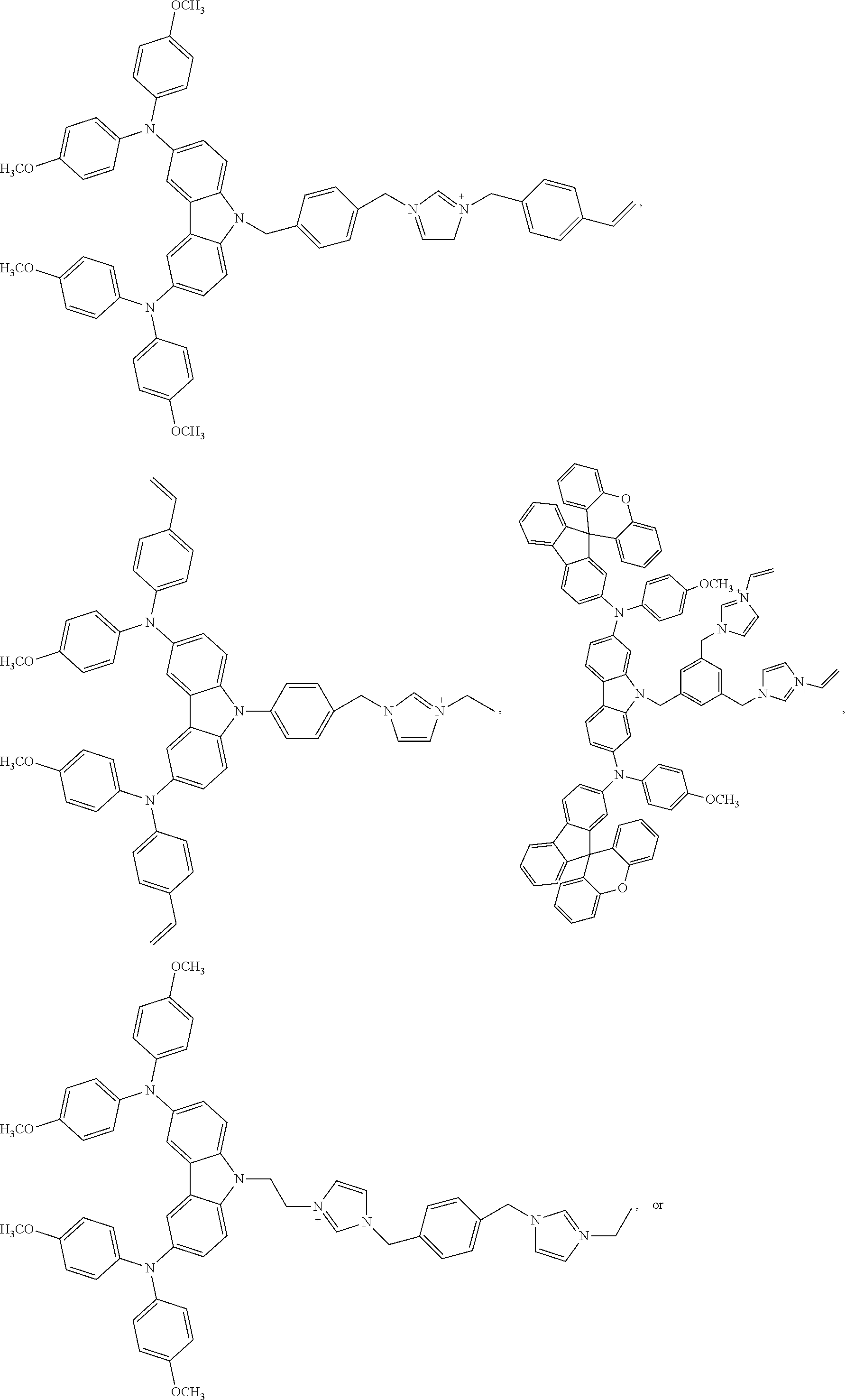

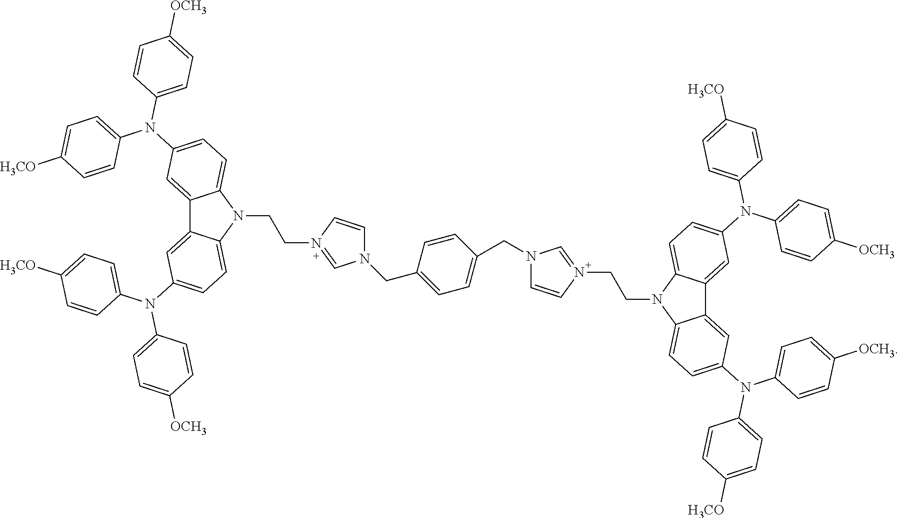

10. The compound of claim 1, having the formula

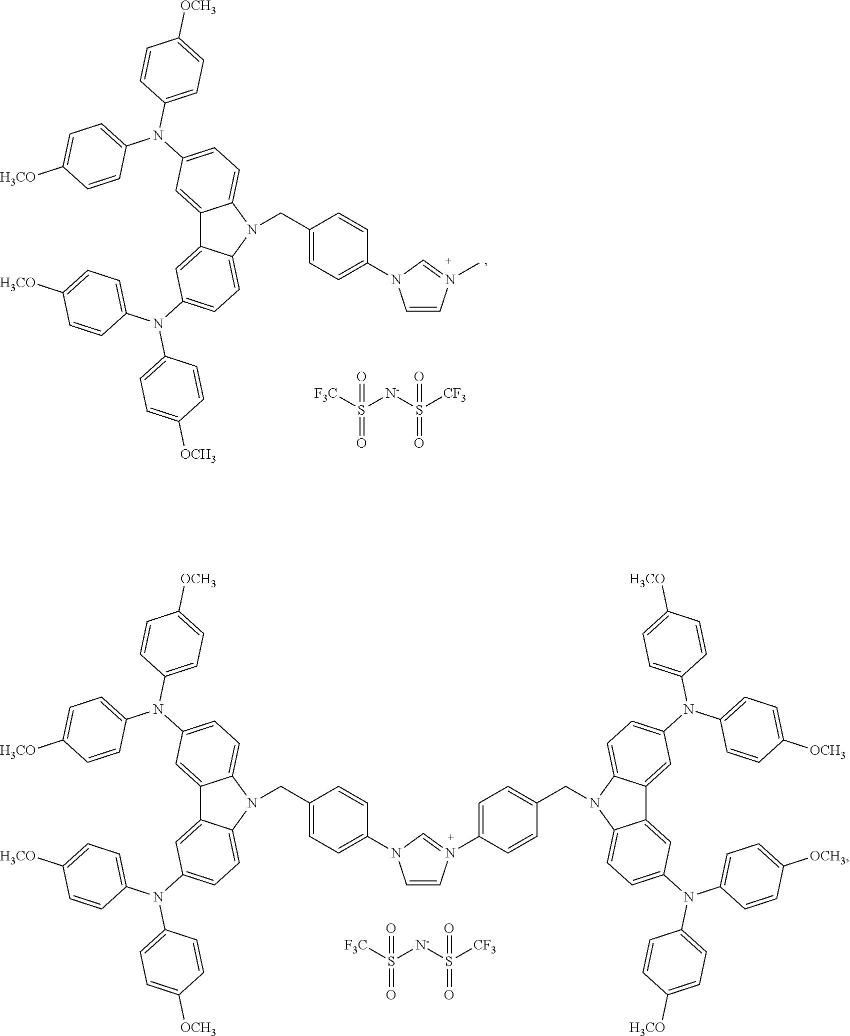

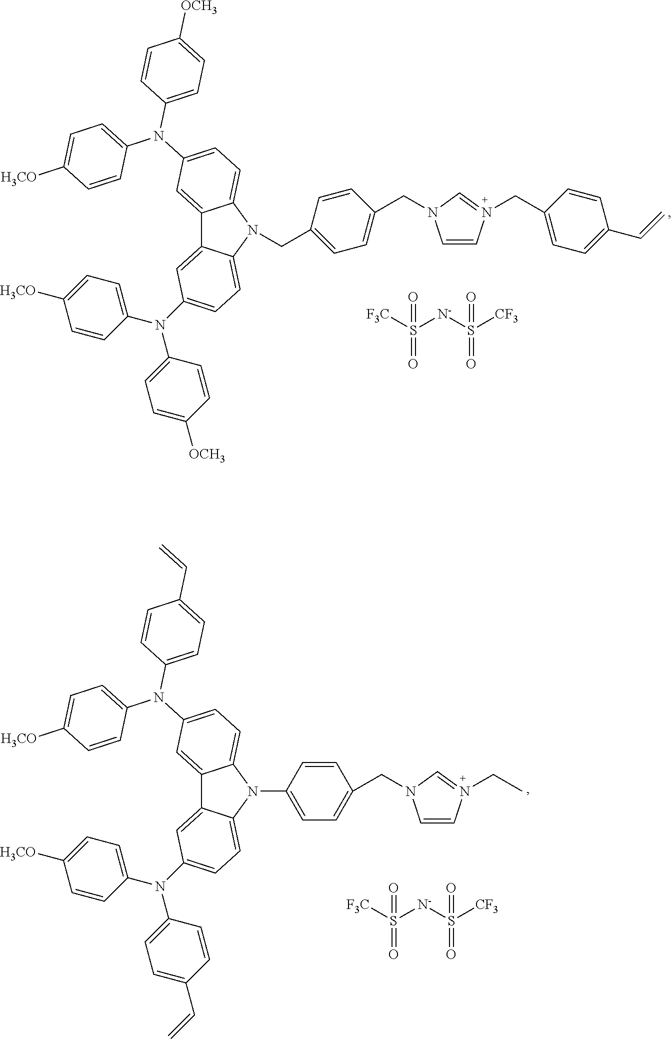

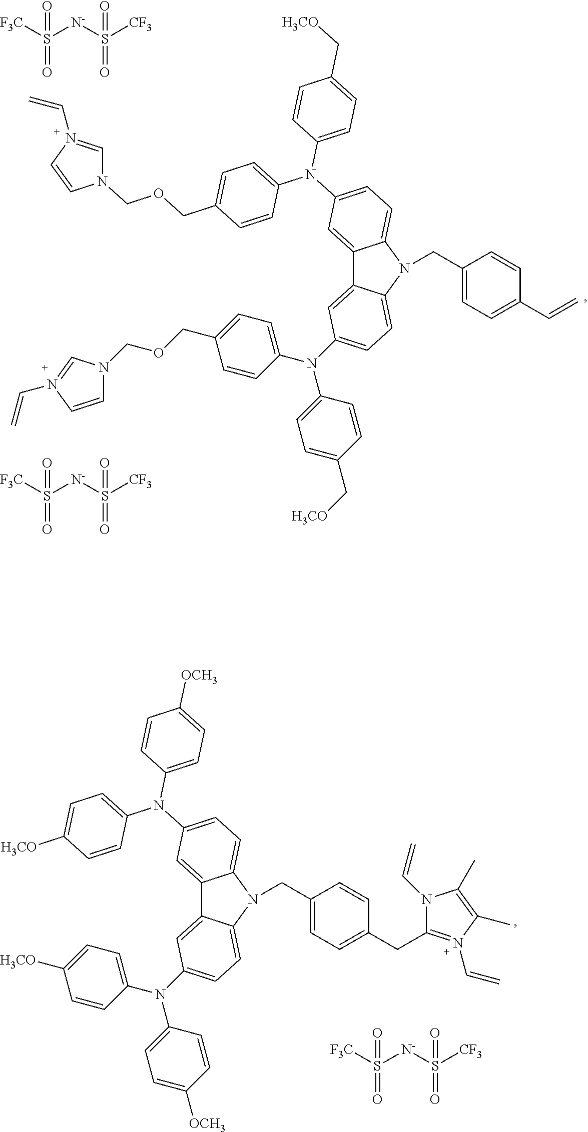

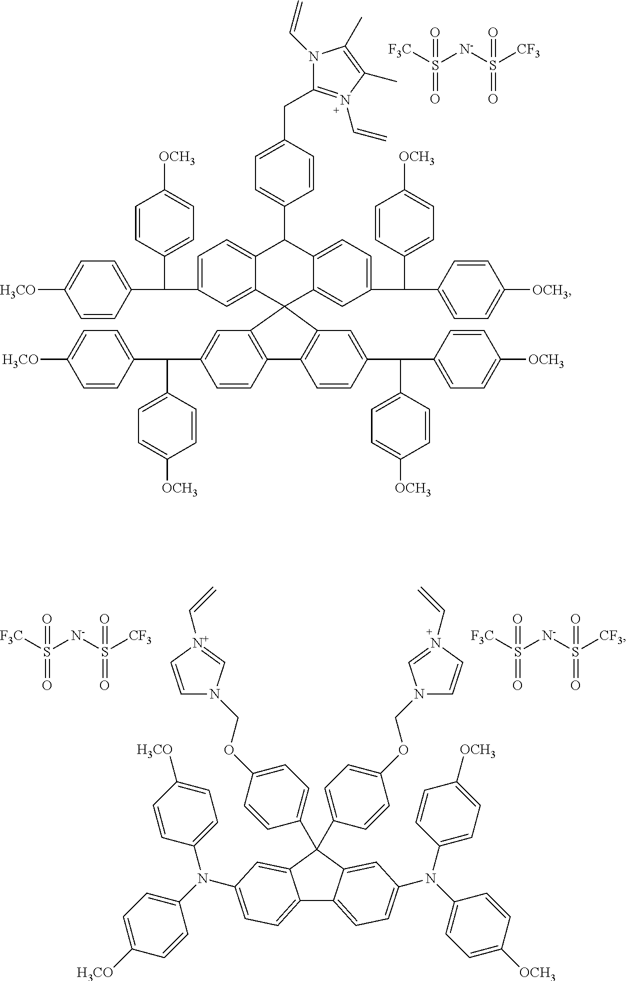

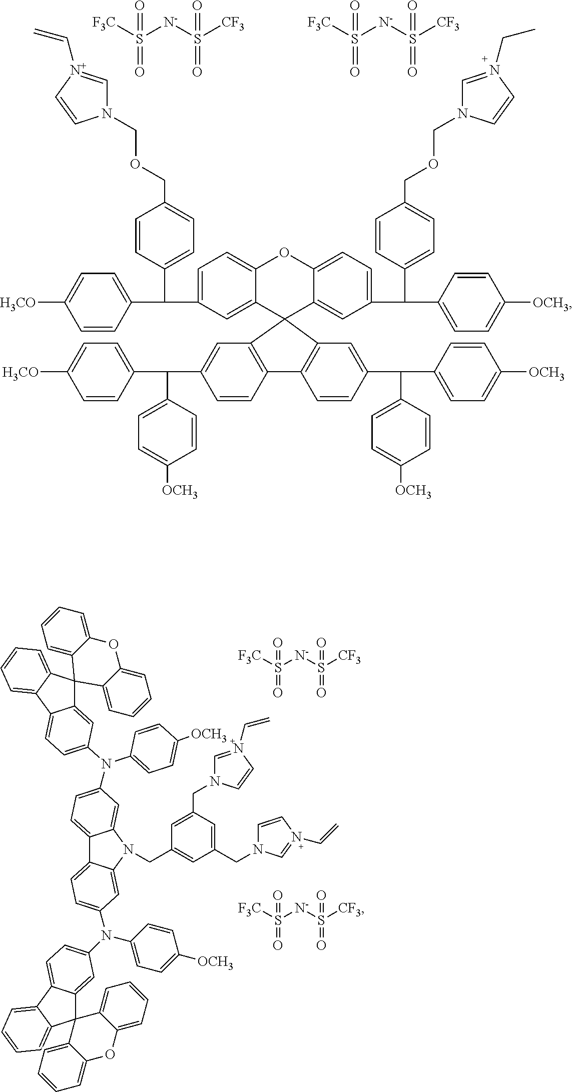

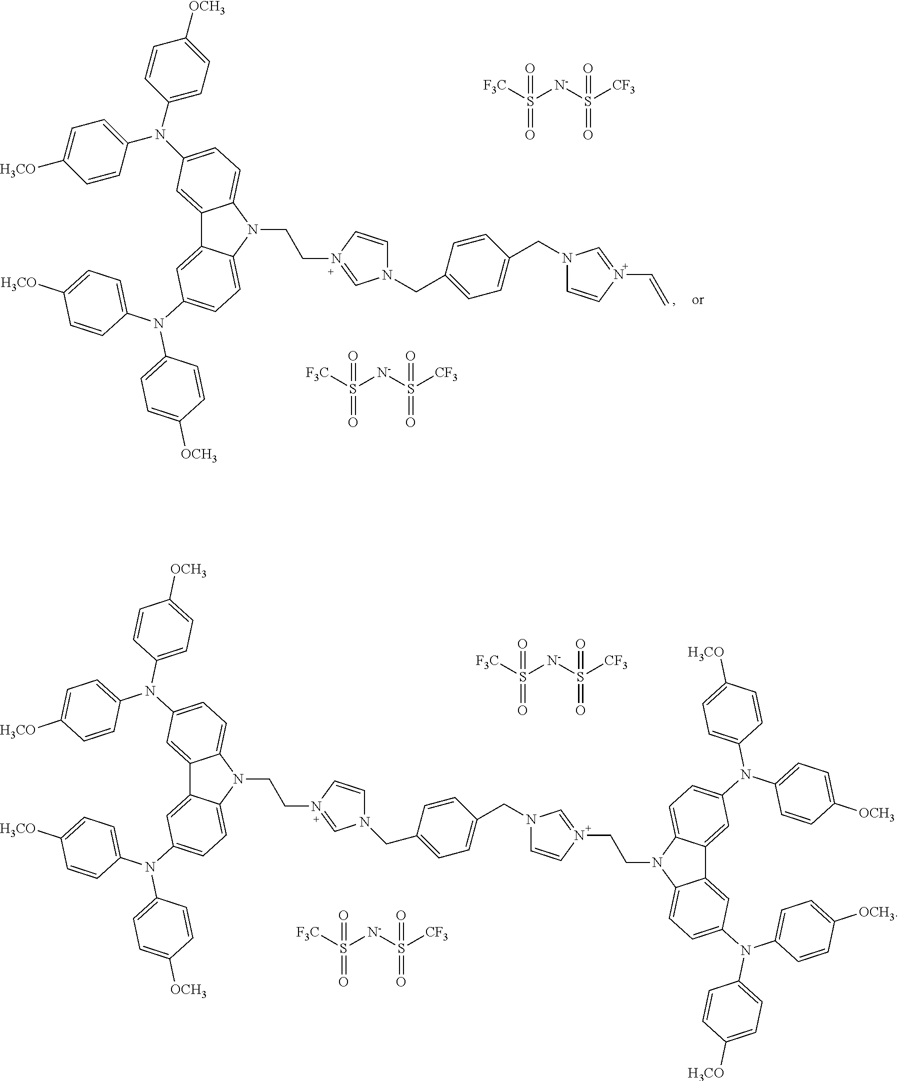

11. A photoactive device comprising:
a first electrode;
a hole transport layer in electrical communication with the electrode, wherein the hole transport layer comprises the compound of claim 1;
a photoactive layer in electrical communication with the hole transport layer; and
a second electrode in electrical communication with the photoactive layer.

12. The photoactive device of claim 11, wherein the photoactive layer includes one or more of:
a material having a perovskite structure;
an organic semiconductor; or
an inorganic semiconductor.

13. The photoactive device of claim 11, further comprising an electron transport layer in electrical communication with the photoactive layer and the second electrode.

14. A mixture comprising:
a lithium-free electrolyte; and
a cross-linkable hole transport compound having a formula:
HTS-L³-R³, wherein:
L³ is a spacer substituent selected from a C₁-C₂₀ branched, unbranched, cyclic, or polycyclic bivalent aliphatic group that is saturated or unsaturated and substituted or unsubstituted; or a bivalent substituted or unsubstituted aromatic group that is fused or unfused; or a second lithium-free electrolyte having a group covalently bonded to HTS and R³;
HTS is a cross-linkable hole transport structure; and
R³ is HTS, H; a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused;
wherein the lithium-free electrolyte comprises a cross-linkable cation component and an anion component, wherein the cross-linkable cation component has a formula of: 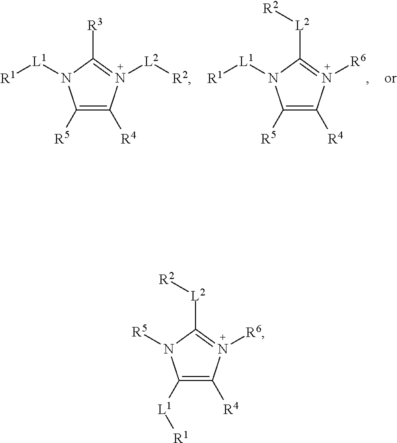
wherein:
R¹, R⁴, R⁵, and R⁶ are independently H; or R²; or a C₁-C₂₀ branched, unbranched, cyclic, or polycyclic monovalent aliphatic group that is saturated or unsaturated and substituted or unsubstituted; or a substituted or unsubstituted monovalent aromatic group that is fused or unfused;
R² is independently a reactive cross-linking group; and
L¹ and L² are independently a spacer substituent selected from a C₁-C₂₀ branched, unbranched, cyclic, or polycyclic bivalent aliphatic group that is saturated or unsaturated and substituted or unsubstituted; or a bivalent substituted or unsubstituted aromatic group that is fused or unfused.

15. The mixture of claim 14, wherein HTS comprises 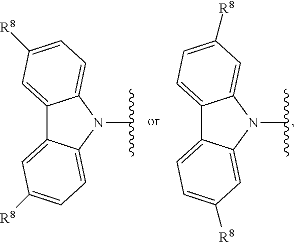
wherein each R⁸ is independently: 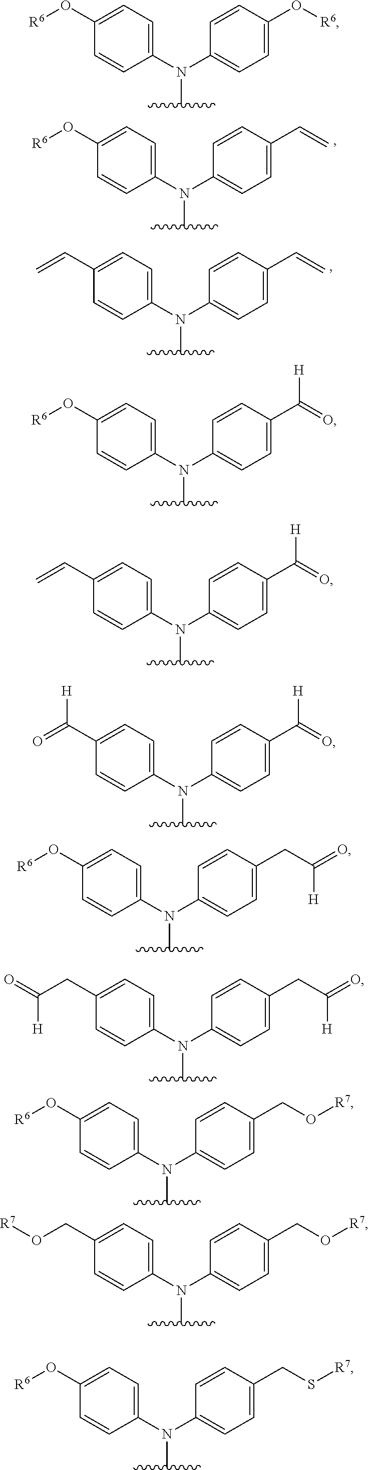 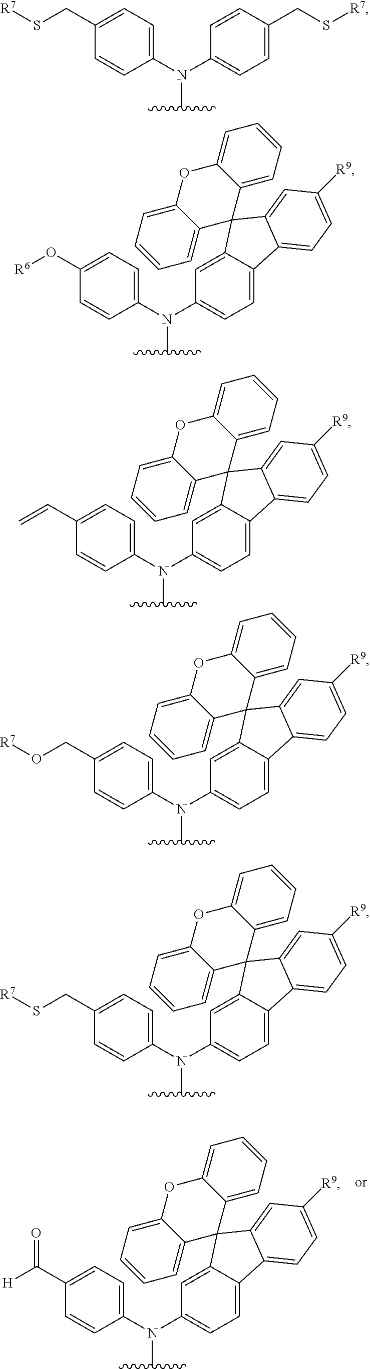 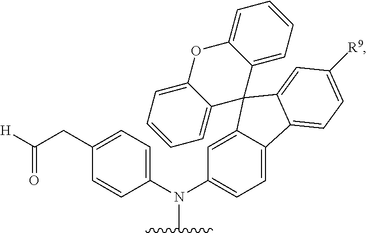
wherein each R⁶ is independently a C₁-C₂₀ branched, unbranched, cyclic, or polycyclic monovalent aliphatic group that is saturated or unsaturated and substituted or unsubstituted, or a substituted or unsubstituted monovalent aromatic group,
wherein each R⁷ is independently a reactive cross-linkable substituent, and
wherein R⁹ is —OR⁶, —R⁷, —OR⁷, or —H.

16. The mixture of claim 14, wherein HTS comprises

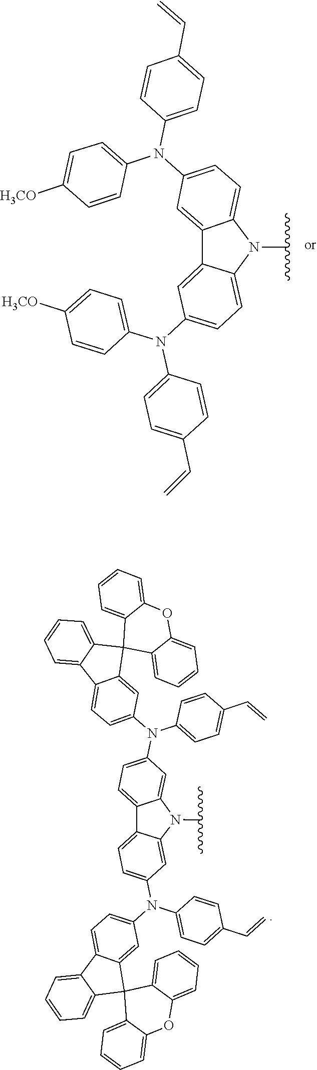 [SMILES: *n1c2ccc(N(c3ccc(C=C)cc3)c3ccc(OC)cc3)cc2c2cc(N(c3ccc(C=C)cc3)c3ccc(OC)cc3)ccc21 ; *n1c2cc(N(c3ccc(C=C)cc3)c3ccc4c(c3)C3(c5ccccc5Oc5ccccc53)c3ccccc3-4)ccc2c2ccc(N(c3ccc(C=C)cc3)c3ccc4c(c3)C3(c5ccccc5Oc5ccccc53)c3ccccc3-4)cc21]

17. The mixture of claim 14, wherein HTS-L³-R³ has a formula of

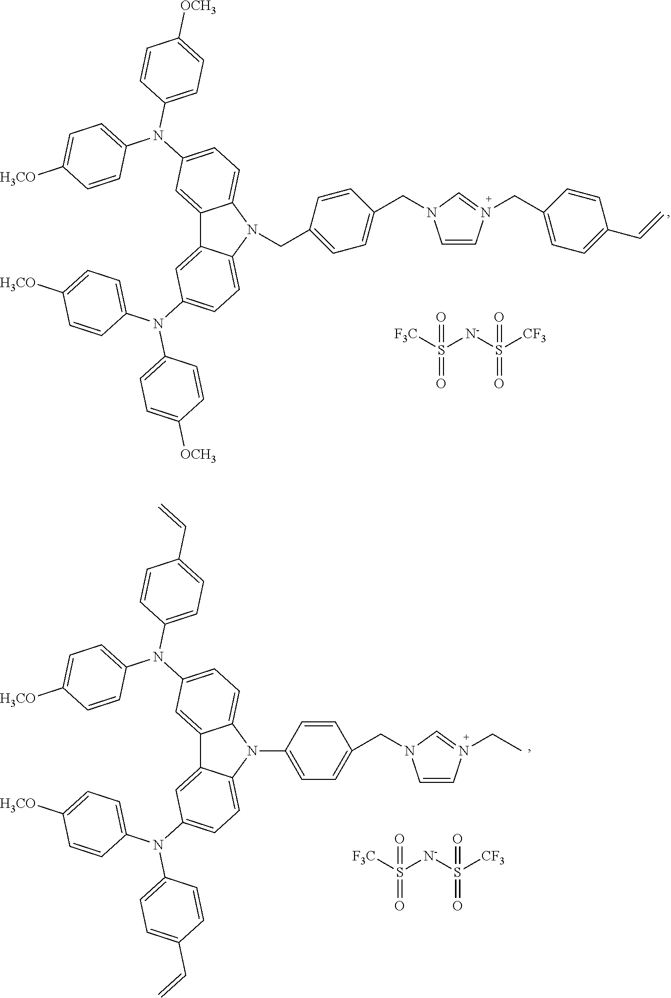

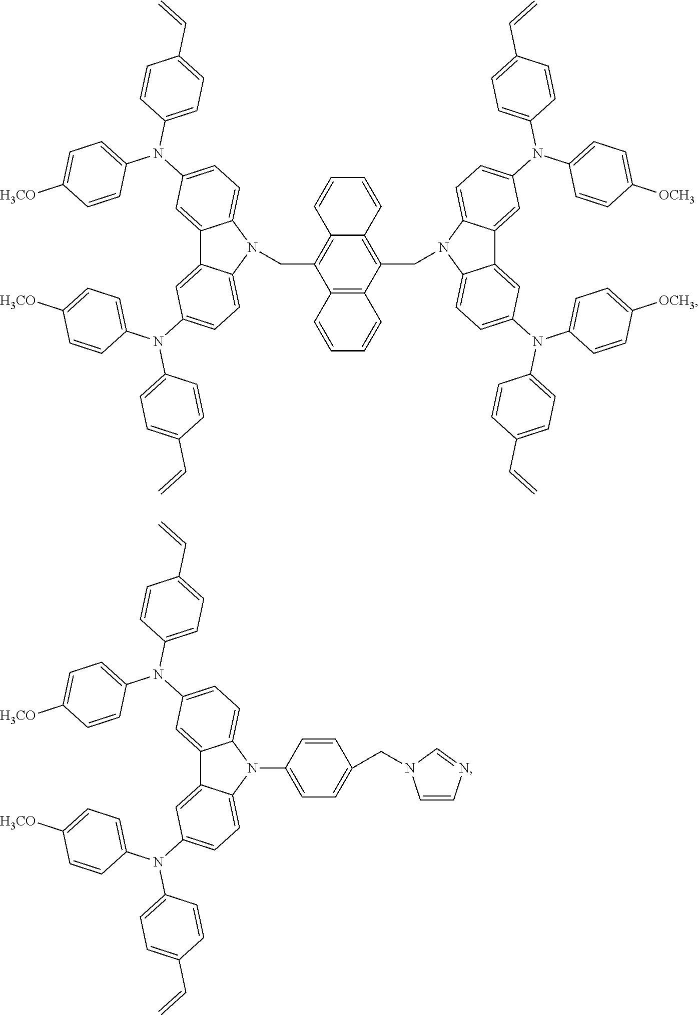

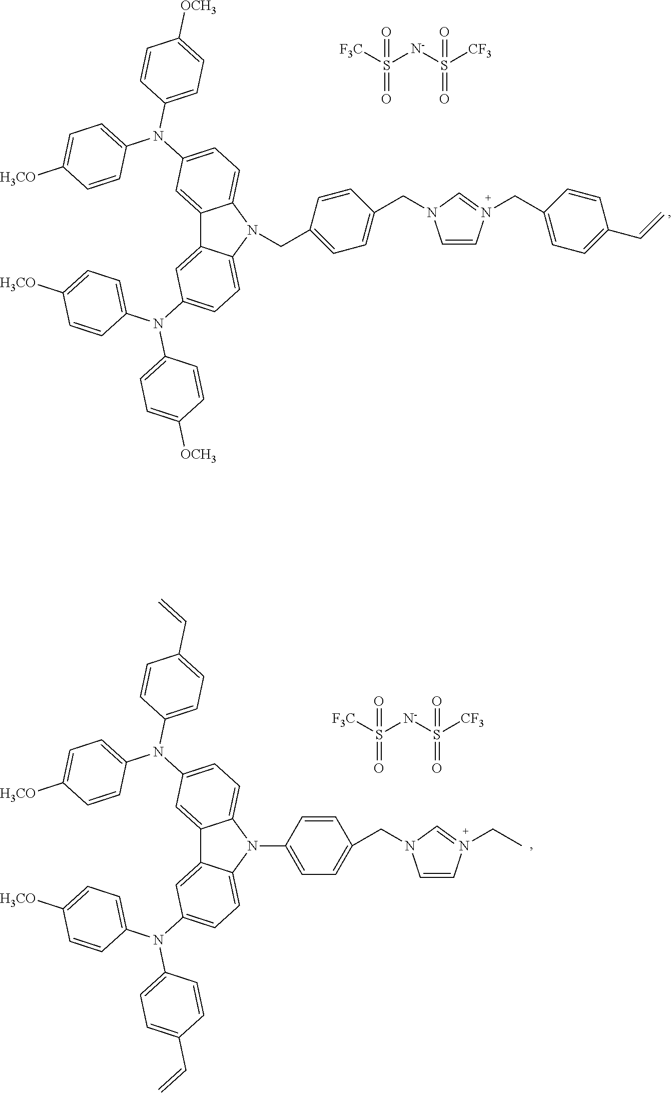

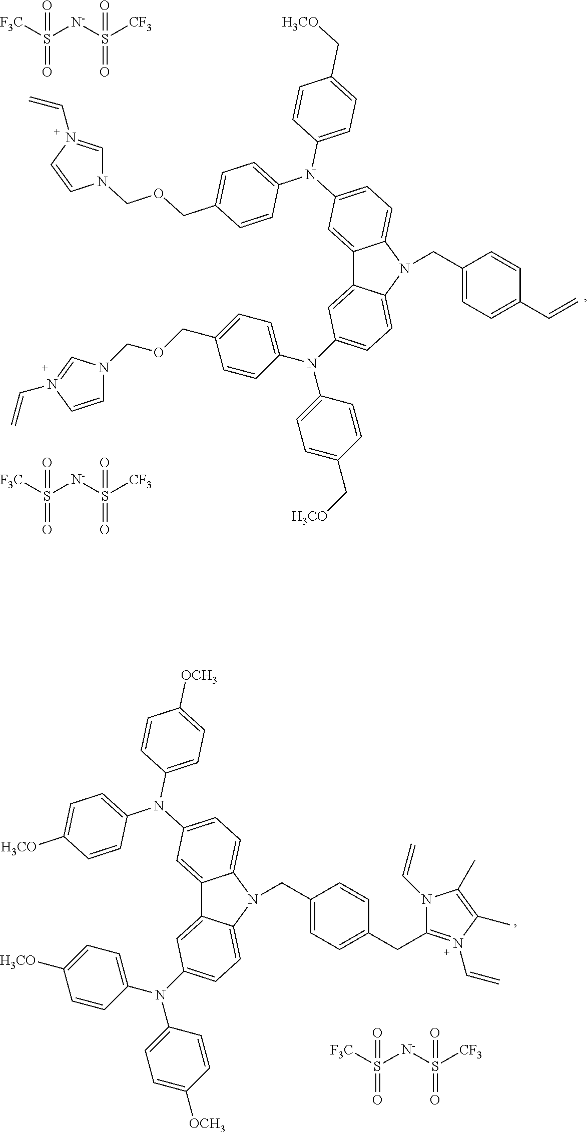

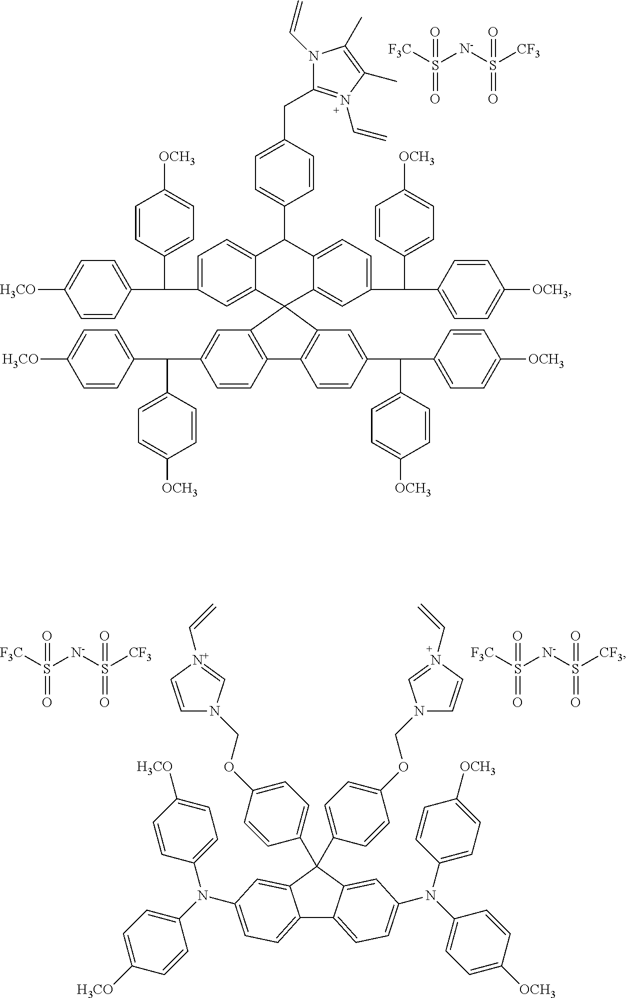

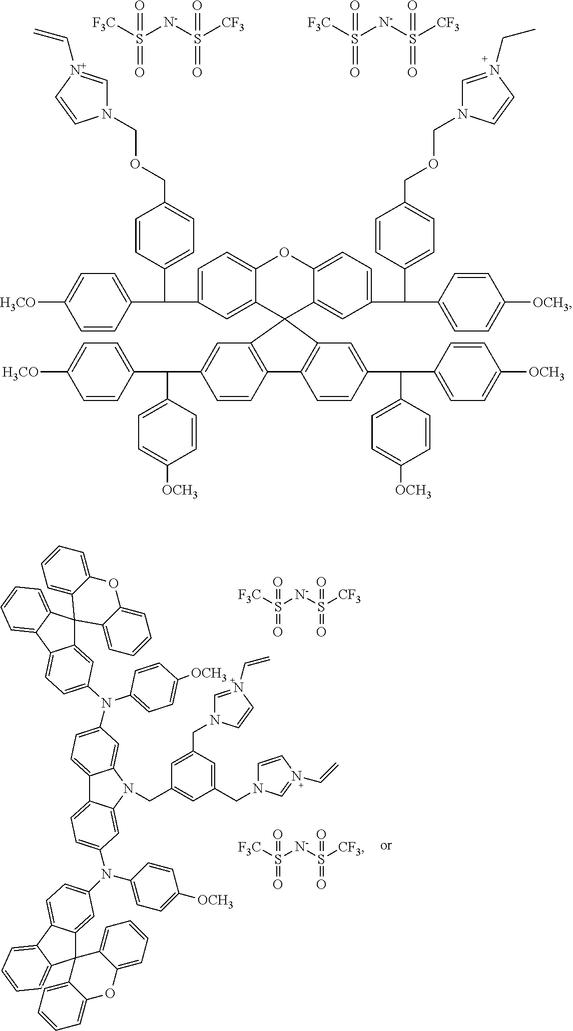

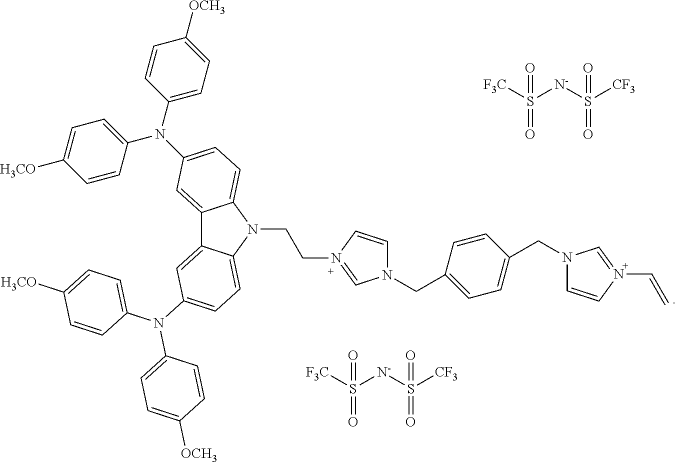

18. A photoactive device comprising:
a first electrode;
a hole transport layer in electrical communication with the electrode, wherein the hole transport layer comprises the mixture of claim 14;
a photoactive layer in electrical communication with the hole transport layer; and
a second electrode in electrical communication with the photoactive layer.

19. The photoactive device of claim 18, wherein the photoactive layer includes one or more of
a material having a perovskite structure;
an organic semiconductor; or
an inorganic semiconductor.

20. The photoactive device of claim 18, further comprising an electron transport layer in electrical communication with the photoactive layer and the second electrode.
